(19) United States
(12) Patent Application Publication
(10) Pub. No.: US 2009/0275567 A1
(43) Pub. Date: Nov. 5, 2009

(54) 2-HETEROCYCLOAMINO-4-IMIDAZOLYLPYRIMIDINES AS AGENTS FOR THE INHIBITION OF CELL PROLIFERATION

(76) Inventors: Clifford Jones, Cheshire (GB); Martin Pass, Cheshire (GB); David Rudge, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/302,367

(22) PCT Filed: May 24, 2007
(86) PCT No.: PCT/GB07/01906

§ 371 Date: Nov. 25, 2008

Related U.S. Application Data
(60) Provisional application No. 60/868,540, filed on Dec. 4, 2006.
(60) Provisional application No. 60/803,283, filed on May 26, 2006.

(51) Int. Cl. A61K 31/506; C07D 401/14; C07D 413/14; C07D 417/14; A61K 31/5377; A61K 31/541; A61P 35/00

(52) U.S. Cl. 514/227.8; 544/328; 544/295; 544/122; 544/56; 514/275; 514/252.19; 514/235.8

(57) ABSTRACT

Compounds of formula (I):

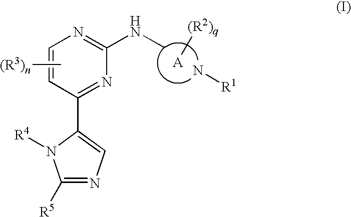

which possess cell-cycle inhibitory activity are described.

The invention relates to pyrimidine derivatives, or pharmaceutically acceptable salts or in vivo hydrolysable esters thereof, which possess cell-cycle inhibitory activity and are accordingly useful for their anti-cell-proliferation (such as anti-cancer) activity and are therefore useful in methods of treatment of the human or animal body. The invention also relates to processes for the manufacture of said pyrimidine derivatives, to pharmaceutical compositions containing them and to their use in the manufacture of medicaments of use in the production of an anti-cell-proliferation effect in a warm-blooded animal such as man.

The cell cycle is fundamental to the survival, regulation and proliferation of cells and is highly regulated to ensure that each step progresses in a timely and orderly manner. The progression of cells through the cell cycle arises from the sequential activation and de-activation of several members of the cyclin-dependent kinase (CDK) family. The activation of CDKs is dependent on their interaction with a family of intracellular proteins called cyclins. Cyclins bind to CDKs and this association is essential for CDK activity within the cell. Different cyclins are expressed and degraded at different points in the cell cycle to ensure that activation and inactivation of CDKs occurs in the correct order for progression through the cell cycle.

Moreover, CDKs appear to be downstream of a number of oncogene signalling pathways. Deregulation of CDK activity by upregulation of cyclins and/or deletion of endogenous inhibitors appears to be an important axis between mitogenic signalling pathways and proliferation of tumour cells.

Accordingly it has been recognised that an inhibitor of cell cycle kinases, particularly inhibitors of CDK1, CDK2, CDK4 and CDK6 (which operate at the G2/M, G1/S—S-G2/M and G1-S phases respectively) should be of value as an active inhibitor of cell proliferation, such as growth of mammalian cancer cells.

Tumour cells are also thought to be highly dependent on the continual transcriptional activity of RNA polymerase II to maintain appropriate levels of anti-apoptotic proteins and ensure tumour cell survival. CDK1, CDK7, CDK8 and CDK9 in particular are known to regulate the activity of RNA polymerase II through phosphorylation of the C-terminal domain of the protein. Thus, the inhibition of RNA polymerase II activity through inhibitors of these CDKs may contribute to a pro-apoptotic effect in tumour cells.

The inhibition of cell cycle kinases is expected to be of value in the treatment of disease states associated with aberrant cell cycles and cell proliferation such as cancers (solid tumours and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

WO 02/20512, WO 03/076435, WO 03/076436, WO 03/076434, WO 03/076433 and WO 04/101549 describe certain 2-anilino-4-imidazolylpyrimidine derivatives that inhibit the effect of cell cycle kinases. The present invention is based on the discovery that a novel group of non-anilino pyrimidines inhibit the effects of CDK2, and thus possess anti-cell-proliferation properties.

Accordingly, the present invention provides a compound of formula (I):

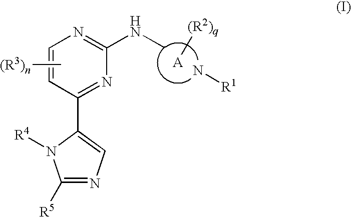

wherein:

Ring A is a 5-7 membered saturated heterocyclic ring which contains one nitrogen atom and optionally 1 or 2 additional heteroatoms selected from N, O or S; wherein 2 atoms of Ring A may optionally be connected by a bridge;

R¹ is a substituent on nitrogen and is selected from hydrogen, C₁₋₆alkyl, C₁₋₆alkanoyl, carbamoyl, N—(C₁₋₆alkyl)carbamoyl, N,N—(C₁₋₆alkyl)carbamoyl, N—(C₁₋₆alkenyl)carbamoyl, N,N—(C₁₋₆alkenyl)carbamoyl, sulphamoyl, N—(C₁₋₆alkyl)sulphamoyl, N,N—(C₁₋₆alkyl)sulphamoyl, N—(C₁₋₆alkenyl)sulphamoyl, N,N—(C₁₋₆alkenyl)₂sulphamoyl, C₁₋₆alkoxycarbonyl, C₁₋₆alkylsulphonyl, C₁₋₆alkenylsulphonyl, carbocyclyl-R⁶ or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;

R² is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;

q is 0-4; wherein the values of R² may be the same or different;

R³ is selected from halo, cyano or amino;

n is 0 to 2, wherein the values of R³ may be the same or different;

R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;

R⁵ is selected from methyl, ethyl, isopropyl, fluoromethyl, difluoromethyl, trifluoromethyl, methoxymethyl, cyclopropylmethyl or cyclopropyl;

R⁶ and R⁷ are independently selected from —C(O)—, —C(O)N—(R¹⁵)—, —S(O)₂— or —SO₂N—(R¹⁶)—; wherein R¹⁵ and R¹⁶ are independently selected from hydrogen or C₁₋₆alkyl;

R⁸ and R¹² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R⁸ and R¹² independently of each other may be optionally substituted on carbon by one or more R¹⁹; and wherein if said heterocyclyl contains an —NH— moiety that nitrogen may be optionally substituted by a group selected from R²⁰;

R⁹, R¹³ and R²⁰ are independently selected from C₁₋₆alkyl, C₁₋₆alkanoyl, C₁₋₆alkylsulphonyl, C₁₋₆alkoxy carbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³ and R²⁰ independently of each other, may be optionally substituted on carbon by one or more R²¹;

R¹⁰, R¹¹, R¹⁷ and R¹⁸ are independently selected from a direct bond, —O—, —N—(R²²)—, —C(O)—, —N—(R²³)C(O)—, —C(O)N—(R²⁴)—, —S(O)ₛ—, —SO₂N—(R²⁵)— or —N(R²⁶)SO₂—; wherein R²², R²³, R²⁴, R²⁵ and R²⁶ are independently selected from hydrogen or C₁₋₆alkyl and s is 0-2;

R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N,N—(C₁₋₆alkyl)amino, N,N—(C₁₋₆allyl)₂-amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino; and

R¹⁹ and R²¹ are independently selected from halo, nitro, cyano, hydroxy, trifluoromethoxy, trifluoromethyl, amino, carboxy, carbamoyl, mercapto, sulphamoyl, methyl, ethyl, propyl, isopropyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

According to a further feature of the present invention there is provided a compound of formula (I) (as depicted above) wherein:

Ring A is a 5-7 membered saturated heterocyclic ring which contains one nitrogen atom and optionally 1 or 2 additional heteroatoms selected from N, O or S; wherein 2 atoms of Ring A may optionally be connected by a bridge;

R¹ is a substituent on nitrogen and is selected from hydrogen, C₁₋₆alkyl, C₁₋₆alkanoyl, carbamoyl, N—(C₁₋₆alkyl)carbamoyl, N,N—(C₁₋₆alkyl)carbamoyl, N—(C₁₋₆alkenyl)carbamoyl, N,N—(C₁₋₆alkenyl)carbamoyl, sulphamoyl, N—(C₁₋₆alkyl)sulphamoyl, N,N—(C₁₋₆alkyl)₂sulphamoyl, N—(C₁₋₆alkenyl)sulphamoyl, N,N—(C₁₋₆alkenyl)₂sulphamoyl, C₁₋₆alkoxycarbonyl, C₁₋₆alkylsulphonyl, C₁₋₆alkenylsulphonyl, carbocyclyl-R⁶ or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;

R² is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;

q is 0-4; wherein the values of R² may be the same or different;

R³ is selected from halo, cyano or amino;

n is 0 to 2, wherein the values of R³ may be the same or different;

R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;

R⁵ is selected from methyl, ethyl, isopropyl, fluoromethyl, difluoromethyl, trifluoromethyl, methoxymethyl, cyclopropylmethyl or cyclopropyl;

R⁶ and R⁷ are independently selected from —C(O)—, —C(O)N—(R¹⁵)—, —S(O)₂— or —SO₂N—(R¹⁶)—; wherein R¹⁵ and R¹⁶ are independently selected from hydrogen or C₁₋₆alkyl;

R⁸ and R¹² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R⁸ and R¹² independently of each other may be optionally substituted on carbon by one or more R¹⁹; and wherein if said heterocyclyl contains an —NH— moiety that nitrogen may be optionally substituted by a group selected from R²⁰;

R⁹, R¹³ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³ and R²⁰ independently of each other, may be optionally substituted on carbon by one or more R²¹;

R¹⁰, R¹¹, R¹⁷ and R¹⁸ are independently selected from a direct bond, —O—, —N—(R²²)—, —C(O)—, —N—(R²³)C(O)—, —C(O)N—(R²⁴)—, —S(O)ₛ—, —SO₂N—(R²⁵)— or —N—(R²⁶)SO₂—; wherein R²², R²³, R²⁴, R²⁵ and R²⁶ are independently selected from hydrogen or C₁₋₆alkyl and s is 0-2;

R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino; and

R¹⁹ and R²¹ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, NH-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; or a pharmaceutically acceptable salt or an in vivo hydrolysable ester thereof.

In this specification the term “alkyl” includes both straight and branched chain alkyl groups but references to individual alkyl groups such as “propyl” are specific for the straight chain version only. For example, “C₁₋₆alkyl” includes methyl, ethyl, propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

Ring A is a “a 5-7 membered saturated heterocyclic ring which contains one nitrogen atom and optionally 1 or 2 additional heteroatoms selected from N, O or S”. A “a 5-7membered saturated heterocyclic ring which contains one nitrogen atom and optionally 1 or 2additional heteroatoms selected from N, O or S” is a saturated heterocyclic ring that contains 5, 6 or 7 atoms of which at least one is a nitrogen atom (to which R¹ is attached) and the others are carbon atoms or carbon atoms and 1 or 2 additional heteroatoms selected from N, O or S; wherein a —CH₂— group can optionally be replaced by a —C(O)—. Particular examples of a “a 5-7 membered saturated heterocyclic ring which contains one nitrogen atom and optionally 1 or 2 additional heteroatoms selected from N, O or S” are piperidin-4-yl, piperazin-1-yl, 3-oxopiperidin-1-yl and pyrrolidin-3-yl.

Two atoms of Ring A may optionally be connected by a bridge. A bridge is a bond, an atom or two atoms connecting two different atoms of Ring A. Where the bridge is one or two atoms the atoms may be independently selected from carbon, nitrogen, sulphur or oxygen. Particularly the bridge is a direct bond. Particularly the bridge is one carbon atom. Alternatively the bridge is two carbon atoms. Alternatively the bridge is a carbon atom and a nitrogen atom. Examples of a “5-7 membered saturated heterocyclic ring which contains one nitrogen atom and optionally 1 or 2 additional heteroatoms selected from N, O or S wherein 2atoms of Ring A” are “connected by a bridge;” include 8-azabicyclo[3.2.1]octan-4-yl, 6,8-diazabicyclo[3.2.1]octan-4-yl, 3-azabicyclo[3.1.0]hex-6-yl, 2-azabicyclo[2.1.0]pent-5-yl, 8-azabicyclo[3.2.1]octan-3-yl or endo-8-azabicyclo[3.2.1]octan-3-yl.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidinyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxopiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)-and a ring sulphur atom may be optionally oxidised to form the S-oxides. For the avoidance of doubt, “heterocyclyl” also includes bridged compounds, defined herein above, for example 7-azabicyclo[2.2.1]heptane and 6-azabicyclo[2.2.2]octane. A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “C₁₋₆alkanoyloxy” include propionyloxy and acetoxy. Examples of “C₁₋₆alkanoylamino” include propionylamino and acetylamino. Examples of “C₂₋₆alkenyl” include vinyl, allyl and 1-propenyl. Examples of “C₂₋₆alkynyl” include ethynyl, 1-propynyl and 2-propynyl. Examples of “N—(C₁₋₆alkyl)sulphamoyl” include N-(methyl)sulphamoyl and N-(ethyl)sulphamoyl. Examples of “N,N—(C₁₋₆alkyl)₂sulphamoyl” include N-(dimethyl)sulphamoyl and N-(methyl)-N-(ethyl)sulphamoyl. Examples of “N—(C₁₋₆alkenyl)sulphamoyl” include N-(allyl)sulphamoyl and N-(ethenyl)sulphamoyl. Examples of “N,N—(C₁₋₆alkenyl)₂sulphamoyl” include N,N-(diallyl)sulphamoyl and N-(allyl)-N-(ethenyl)sulphamoyl. Examples of “N—(C₁₋₆alkyl)carbamoyl” include methylaminocarbonyl and ethylaminocarbonyl. Examples of “N,N—(C₁₋₆alkyl)₂carbamoyl” include dimethylaminocarbonyl and methylethylaminocarbonyl. Examples of “N—(C₁₋₆alkenyl)carbamoyl” include alkylaminocarbonyl and ethenylaminocarbonyl. Examples of “N,N—(C₁₋₆alkenyl)₂carbamoyl” include diallylaminocarbonyl and (allyl)(ethenyl)aminocarbonyl. Examples of “C₁₋₆alkylsulphonyl” include methylsulphonyl and isopropylsulphonyl. Examples of “C₁₋₆alkenylsulphonyl” include alkylsulphonyl and ethenylsulphonyl. Examples of “C₁₋₆alkylsulphonylamino” include mesylamino and isopropylsulphonylamino. Examples of “N,N—(C₁₋₆alkyl)₂amino” include methylamino and ethylamino. Examples of “N,N—(C₁₋₆alkyl)₂amino” include di-N-methylamino, di-(N-ethyl)amino and N-ethyl-N-methylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have chiral centres and/or geometric isomeric centres (E- and Z-isomers), and it is to be understood that the invention encompasses all such optical, diastereoisomers and geometric isomers that possess CDK inhibitory activity.

The invention relates to any and all tautomeric forms of the compounds of the formula (I) that possess CDK inhibitory activity.

It is also to be understood that certain compounds of the formula (I) can exist in solvated as well as unsolvated forms such as, for example, hydrated forms. It is to be understood that the invention encompasses all such solvated forms which possess CDK inhibitory activity.

Particular values of variable groups are as follows. Such values may be used where appropriate with any of the definitions, claims or embodiments defined hereinbefore or hereinafter.

Ring A is a 5 membered saturated heterocyclic ring which contains one nitrogen atom and optionally 1 or 2 additional heteroatoms selected from N, O or S; wherein 2 atoms of Ring A may optionally be connected by a bridge.

Ring A is a 6 membered saturated heterocyclic ring which contains one nitrogen atom and optionally 1 or 2 additional heteroatoms selected from N, O or S; wherein 2 atoms of Ring A may optionally be connected by a bridge.

Ring A is a 7 membered saturated heterocyclic ring which contains one nitrogen atom and optionally 1 or 2 additional heteroatoms selected from N, O or S; wherein 2 atoms of Ring A may optionally be connected by a bridge.

Ring A is a 5 or 6 membered saturated heterocyclic ring which contains one nitrogen atom; wherein 2 atoms of Ring A may optionally be connected by a bridge.

Ring A is a 5-7 membered saturated heterocyclic ring which contains one nitrogen atom; wherein 2 atoms of Ring A may optionally be connected by a bridge.

Ring A is piperidin-4-yl or pyrrolidin-3-yl; wherein 2 atoms of Ring A may optionally be connected by a two carbon atom bridge.

Ring A is piperidin-3-yl, piperidin-4-yl or pyrrolidin-3-yl; wherein 2 atoms of Ring A may optionally be connected by a bond or a two carbon atom bridge.

Ring A is azepan-3-yl, piperidin-3-yl, piperidin-4-yl or pyrrolidin-3-yl; wherein 2atoms of Ring A may optionally be connected by a bond or a two carbon atom bridge.

Ring A is piperidin-4-yl, pyrrolidin-3-yl or 8-azabicyclo[3.2.1]octan-3-yl.

Ring A is 3-azabicyclo[3.1.0]hexan-3-yl, piperidin-3-yl, piperidin-4-yl, pyrrolidin-3-yl or 8-azabicyclo[3.2.1]octan-3-yl.

Ring A is azepan-3-yl, 3-azabicyclo[3.1.0]hexan-3-yl, piperidin-3-yl, piperidin-4-yl, pyrrolidin-3-yl or 8-azabicyclo[3.2.1]octan-3-yl.

Ring A is piperidin-4-yl, pyrrolidin-3-yl or endo-8-azabicyclo[3.2.1]octan-3-yl.

Ring A is (1α, 5α, 6α)-3-azabicyclo[3.1.0]hexan-3-yl, (R)-piperidin-3-yl, (S)-piperidin-3-yl, piperidin-4-yl, pyrrolidin-3-yl or endo-8-azabicyclo[3.2.1]octan-3-yl.

Ring A is azepan-3-yl, (1α, 5α, 6α)-3-azabicyclo[3.1.0]hexan-3-yl, (R)-piperidin-3-yl, (S)-piperidin-3-yl, piperidin-4-yl, pyrrolidin-3-yl or endo-8-azabicyclo[3.2.1]octan-3-yl.

R¹ is a substituent on nitrogen and is selected from hydrogen, C₁₋₆alkyl, C₁₋₆alkanoyl, sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸; wherein

R⁷ is —C(O)—;

R⁸ is selected from halo, hydroxy, C₁₋₆alkyl, C₁₋₆alkoxy, N—(C₁₋₆alkyl)amino, carbocyclyl-R¹⁷— or heterocyclyl-R¹⁸—; wherein R⁸ may be optionally substituted on carbon by one or more R¹⁹; and wherein if said heterocyclyl contains an —NH— moiety that nitrogen may be optionally substituted by a group selected from R²⁰;

R²⁰ is selected from C₁₋₆alkyl or C₁₋₆alkoxycarbonyl; wherein R²⁰ may be optionally substituted on carbon by one or more R²¹;

R¹⁷ and R¹⁸ are a direct bond; and

R¹⁹ and R²¹ are independently selected from hydroxy and methoxy.

R¹ is a substituent on nitrogen and is selected from hydrogen, C₁₋₆alkyl, C₁₋₆alkanoyl, carbamoyl, N—(C₁₋₆alkyl)carbamoyl, N,N—(C₁₋₆alkyl)carbamoyl, sulphamoyl, (C₁₋₆alkyl)sulphamoyl, N,N—(C₁₋₆alkyl)₂sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;

R⁷ is selected from —C(O)—, —C(O)N—(R¹⁵)—, —S(O)₂— or —SO₂N—(R¹⁶)—; wherein R¹⁵ and R¹⁶ are hydrogen;

R⁸ is selected from halo, nitro, hydroxy, amino, C₁₋₆alkyl, C₁₋₆alkoxy, N—(C₁₋₆alkyl)amino, N,N—(C₁₋₆alkyl)₂amino, carbocyclyl-R¹⁷— or heterocyclyl-R¹⁸—; wherein R⁸may be optionally substituted on carbon by one or more R¹⁹; and wherein if said heterocyclyl contains an —NH— moiety that nitrogen may be optionally substituted by a group selected from R²⁰;

R⁹ and R²⁰ are independently selected from C₁₋₆alkyl, C₁₋₆alkanoyl, C₁₋₆alkoxycarbonyl and benzyloxycarbonyl; wherein R⁹ and R²⁰ independently of each other, may be optionally substituted on carbon by one or more R²¹;

R¹⁷ and R¹⁸are independently selected from a direct bond or —N—(R²²)—; wherein R²²is selected from hydrogen or C₁₋₆alkyl; and

R¹⁹ and R²¹are independently selected from halo, cyano, hydroxy, carbamoyl, methyl, propyl, cyclopropyl and methoxy.

R¹is a substituent on nitrogen and is selected from hydrogen, methyl, ethyl, propyl, isopropyl, acetyl, propanoyl, butanoyl, sulphamoyl, methoxycarbonyl, ethoxycarbonyl, t-butoxycarbonyl, mesyl, ethylsulphonyl, propylsulphonyl, ethenylsulphonyl or piperidinyl-R⁷; wherein R¹may be optionally substituted on carbon by one or more R⁸; wherein

R⁷ is —C(O)—;

R⁸ is selected from chloro, hydroxy, methyl, methoxy, ethylamino, isopropylamino, propylamino, but-2-ylamino, t-butylamino, 3-methylbut-2-ylamino, phenyl-R¹⁷—, piperazinyl-R¹⁸—, piperidinyl-R¹⁸—, morpholino-R¹⁸— or pyrrolidinyl-R¹⁸—; wherein R⁸ may be optionally substituted on carbon by one or more R¹⁹; and wherein said piperidinyl or piperazinyl may be optionally substituted on nitrogen by a group selected from R²⁰;

R²⁰ is selected from methyl, ethyl or t-butoxycarbonyl; wherein R²⁰ may be optionally substituted on carbon by one or more R²¹;

R¹⁷ and R¹⁸ are a direct bond; and

R¹⁹ and R²¹ are independently selected from hydroxy and methoxy.

R¹ is a substituent on nitrogen and is selected from hydrogen, methyl, ethyl, propyl, isopropyl, acetyl, propionyl, butanoyl, carbamoyl, N-methylcarbamoyl, N-ethylcarbamoyl, N,N-dimethylcarbamoyl, sulphamoyl, N-ethylsulphamoyl, N-propylsulphamoyl, N-(2,2-dimethylpropyl)sulphamoyl, N-methyl-N-propylsulphamoyl, N,N-dimethylsulphamoyl, methoxycarbonyl, ethoxycarbonyl, t-butoxycarbonyl, mesyl, ethylsulphonyl, propylsulphonyl, 3-methylbutylsulphonyl, ethenylsulphonyl, morpholinyl-R⁷, homopiperazinyl-R⁷, piperazinyl-R⁷, pyrrolidinyl-R⁷ or piperidinyl-R⁷; wherein R¹ may be optionally substituted on carbon by one or more R⁸; and wherein said morpholinyl, homopiperazinyl, piperazinyl, piperidinyl or pyrrolidinyl may be optionally substituted by a group selected from R⁹;

R⁷ is selected from —C(O)—, —C(O)N—(R¹⁵)—, —S(O)₂— or —SO₂N—(R¹⁶)—; wherein R¹⁵and R¹⁶ are hydrogen;

R⁸ is selected from chloro, nitro, hydroxy, amino, methyl, methoxy, N-methylamino, N-ethylamino, N-propylamino, N-isopropylamino, N-but-2-ylamino, 3-methylbut-2-ylamino, 2-methylprop-2-ylamino, N,N-dimethylamino, N,N-diethylamino, N-methyl-N-ethylamino, N-methyl-N-isopropylamino, cyclopentyl-R¹⁷—, cyclobutyl-R¹⁷—, cyclopropyl-R¹⁷—, phenyl-R¹⁷—, morpholino-R¹⁸—, homomorpholino-R¹⁸—, thiomorpholino-R¹⁸—, homothiomorpholino-R¹⁸—, piperidinyl-R¹⁸—, 7-azabicyclo[2.2.1]heptyl-R¹⁸—, 2-azabicyclo[2.2.2]octyl-R¹⁸—, 6-azabicyclo[2.2.2]octyl-R¹⁸—, azetidinyl-R¹⁸—, pyrrolidinyl-R¹⁸—, 2-oxopiperazinyl-R¹⁸—, 2-oxohomopiperazinyl-R¹⁸—, homopiperazinyl-R¹⁸— or piperazinyl-R¹⁸—; wherein R⁸ may be optionally substituted on carbon by one or more R¹⁹; and wherein said homopiperazinyl, pyrrolidinyl, piperazinyl or piperidinyl may be optionally substituted on nitrogen by a group selected from R²⁰;

R⁹ and R²⁰ are independently selected from methyl, ethyl, isopropyl, acetyl, t-butoxycarbonyl and benzyloxycarbonyl; wherein R⁹ and R²⁰ independently of each other, may be optionally substituted on carbon by one or more R²¹;

R¹⁷ and R¹⁸ are independently selected from a direct bond or —N—(R²²)—; wherein R²² is selected from hydrogen or methyl; and

R¹⁹ and R²¹ are independently selected from fluoro, cyano, hydroxy, carbamoyl, methyl, propyl, cyclopropyl and methoxy.

R¹ is a substituent on nitrogen and is selected from hydrogen, methyl, propyl, isopropyl, ethenylsulphonyl, mesyl, benzyloxycarbonyl, t-butoxycarbonyl, acetyl, phenethyl, ethoxycarbonyl, 2-methoxyethyl, sulphamoyl, benzyl, 3-chloropropylsulphonyl, 3-(2-methoxyethylamino)propylsulphonyl, 3-hydroxypropylsulphonyl, 3-(1-hydroxyprop-2-ylamino)propylsulphonyl, 3-(1-methylpiperazin-4-yl)propylsulphonyl, 3-[1-(2-hydroxyethyl)piperazin-4-yl]propylsulphonyl, 3-pyrrolidin-1-ylpropylsulphonyl, 3-morpholinopropylsulphonyl, 3-(1-hydroxybut-2-ylamino)propylsulphonyl, 3-(1-methoxyprop-2-ylamino)propylsulphonyl, 3-(2-hydroxypropylamino)propylsulphonyl, 3-(1-hydroxy-3-methylbut-2-ylamino)propylsulphonyl, 4-morpholinobutanoyl, 3-(1-hydroxy-2-methylprop-2-ylamino)propylsulphonyl, 3-(piperazin-4-yl)propanoyl, 2-(1-methylpiperazin-4-yl)ethylsulphonyl, 2-pyrrolidin-1-ylethylsulphonyl, 2-(2-methoxyethylamino)ethylsulphonyl, 2-(1-t-butoxycarbonylpiperidin-4-yl)acetyl, 3-(1-t-butoxycarbonylpiperazin-4-yl)propanoyl, 2-(piperidin-4-yl)acetyl, 3-(1-t-butoxycarbonylpiperidin-4-yl)propanoyl, 2-(1-t-butoxycarbonylpiperidin-3-yl)acetyl, 1-t-butoxycarbonyl-4-methylpiperidin-4-ylcarbonyl, 3-(piperidin-4-yl)propanoyl, 4-methylpiperidin-4-ylcarbonyl and 2-(piperidin-3-yl)acetyl.

R¹ is a substituent on nitrogen and is selected from hydrogen, methyl, propyl, isopropyl, ethenylsulphonyl, mesyl, benzyloxycarbonyl, t-butoxycarbonyl, acetyl, phenethyl, ethoxycarbonyl, 2-methoxyethyl, sulphamoyl, N-dimethylsulphamoyl, N,N-dimethylcarbamoyl, benzyl, carbamoyl, N-methylcarbamoyl, 2-(dimethylamino)ethylsulphonyl, 2-(N-methyl-N-isopropyl)ethylsulphonyl, 2-(1-methylpiperazin-4-yl)ethylsulphonyl, 2-pyrrolidin-1-ylethylsulphonyl, 2-(3-fluoropyrrolidin-1-yl)ethylsulphonyl, 2-(thiomorpholin-4-yl)ethylsulphonyl, 2-(4-methylpiperidin-1-yl)ethylsulphonyl, 2-(homopiperidin-1-yl)ethylsulphonyl, 2-diethylaminoethylsulphonyl, 2-azetidin-1-ylethylsulphonyl, 2-morpholinoethylsulphonyl, 2-(4-fluoropiperidin-1-yl)ethylsulphonyl, 2-(4-cyanopiperidin-1-yl)ethylsulphonyl, 2-(4-propylpiperidin-1-ylethylsulphonyl, 2-(4-carbamoylpiperidin-1-ylethylsulphonyl, 2-(7-azabicyclo[2.2.1]hept-7-yl)ethylsulphonyl, 2-(2-azabicyclo[2.2.2]oct-2-yl)ethylsulfonyl, 2-(6-azabicyclo[2.2.2]oct-6-yl)ethylsulfonyl, 2-homomorpholinoethylsulphonyl, 2-(2-oxopiperazin-4-yl)ethylsulphonyl, 2-(1-acetylpiperazin-4-yl)ethylsulphonyl, 2-(2-methoxyethylamino)ethylsulphonyl, 2-(N-methyl-N-cyclopropyl)ethylsulphonyl, 2-(2-oxohomopiperazin-4-yl)ethylsulphonyl, 2-(1-acetylhomopiperazin-4-yl)ethylsulphonyl, 2-(N-methyl-N-cyclopropylmethyl)ethylsulphonyl, 2-(homothiomorpholin-4-yl)ethylsulphonyl, 3-chloropropylsulphonyl, 3-dimethylaminopropylsulphonyl, 3-dimethylamino-2,2-dimethylpropylsulphonyl, 3-diethylaminopropylsulphonyl, 3-(2-methoxyethylamino)propylsulphonyl, 3-[N-methyl-N-(2-methoxyethyl)amino]propylsulphonyl, 3-hydroxypropylsulphonyl, 3-(1-hydroxyprop-2-ylamino)propylsulphonyl, 3-(1-methylpiperazin-4-yl)propylsulphonyl, 3-(1-isopropylpiperazin-4-yl)propylsulphonyl, 3-(6-azabicyclo[2.2.2]oct-6-yl)propylsulfonyl, 3-(7-azabicyclo[2.2.1]hept-7-yl)propylsulphonyl, 3-[1-(2-hydroxyethyl)piperazin-4-yl]propylsulphonyl, 3-pyrrolidin-1-ylpropylsulphonyl, 3-(1,4-dimethylpyrrolidin-1-yl)propylsulphonyl, 3-morpholinopropylsulphonyl, 3-(1-hydroxybut-2-ylamino)propylsulphonyl, 3-(1-methoxyprop-2-ylamino)propylsulphonyl, 3-(2-hydroxypropylamino)propylsulphonyl, 3-(1-hydroxy-3-methylbut-2-ylamino)propylsulphonyl, 3-(1-hydroxy-2-methylprop-2-ylamino)propylsulphonyl, 3-(piperidin-1-yl)propylsulphonyl, 3-(cyclopropylamino)propylsulphonyl, 3-(N-methyl-N-cyclopropylamino)propylsulphonyl, 3-(cyclopentylamino)propylsulphonyl, 3-(N-methyl-N-cyclopentylamino)propylsulphonyl, 3-(cyclobutylamino)propylsulphonyl, 3-(N-methyl-N-cyclobutylamino)propylsulphonyl, 3-(isopropylamino)propylsulphonyl, 3-(N-methyl-N-isopropylamino)propylsulphonyl, 3-(N-methyl-N-ethylamino)propylsulphonyl, 3-[N-methyl-N-(2-cyanoethyl)amino]propylsulphonyl, 3-[N-ethyl-N-(2-cyanoethyl)amino]propylsulphonyl, 3-azetidin-1-ylpropylsulphonyl, 3-(cyclopropylmethylamino)propylsulphonyl, 3-(N-methyl-N-cyclopropylmethylamino)propylsulphonyl, 3-nitro-3-methylbutylsulphonyl, 3-amino-3-methylbutylsulphonyl, 3-dimethyl-3-methylbutylsulphonyl, 2-(piperidin-3-yl)acetyl, 2-(1-t-butoxycarbonylpiperidin-3-yl)acetyl, 2-(piperidin-4-yl)acetyl, 2-(1-f-butoxycarbonylpiperidin-4-yl)acetyl, 2-dimethylaminoacetyl, 3-(1-t-butoxycarbonylpiperazin-4-yl)propanoyl, 3-(1-t-butoxycarbonylpiperidin-4-yl)propanoyl, 3-(piperidin-4-yl)propanoyl, 3-(piperazin-4-yl)propanoyl, 3-dimethylaminopropanol, 4-morpholinobutanoyl, 4-dimethylaminobutanoyl, 1-f-butoxycarbonyl-4-methylpiperidin-4-ylcarbonyl, 4-methylpiperidin-4-ylcarbonyl, 1-t-butoxycarbonyl-4-methylpiperidin-4-ylcarbonyl, 4-methylhomopiperazin-1-ylcarbonyl, 1-methylpiperidin-3-ylcarbonyl, 1-methylpyrrolidin-2-ylcarbonyl, 3-dimethylaminopyrrolidin-1-ylcarbonyl, 4-t-butoxycarbonylmorpholin-2-ylcarbonyl, morpholin-2-ylcarbonyl, 1-methylpiperidin-4-ylcarbamoyl, N-(1-ethylpyrrolidin-2-ylmethyl)carbamoyl, N-(2-pyrrolidin-1-ylethyl)carbamoyl, N-(2-dimethylaminoethyl)carbamoyl, N-(1-methylpiperidin-4-yl)sulphamoyl, N-(1-isopropylpiperidin-4-yl)sulphamoyl, 2-(dimethylamino)ethylsulphamoyl, 2-(diethylamino)ethylsulphamoyl, 2-(morpholino)ethylsulphamoyl, 2-(1-methylpiperazin-4-yl)ethylsulphamoyl, 2-(1-methylpyrrolidin-2-yl)ethylsulphamoyl, 3-(pyrrolidin-1-yl)propylsulphamoyl, 3-(3-fluoropyrrolidin-1-yl)propylsulphamoyl, 3-dimethylamino-2,2-dimethylpropylsulphamoyl, 3-(piperidin-1-yl)propylsulphamoyl, N-methyl-N-(3-dimethylaminopropyl)sulphamoyl, 3-dimethylaminopyrrolidin-1-ylsulphonyl, 1-methylpiperazin-4-ylsulphonyl, 1-methylpiperidin-4-ylsulphonyl, 1-isopropylpiperidin-4-ylsulphonyl and 1-methylhomopiperazin-4-ylsulphonyl.

q is 0.

R³ is halo.

R³ is fluoro or chloro.

n is 0.

n is 1.

n is 0 or 1.

R⁴ is isopropyl.

R⁴ is cyclopentyl.

R⁴ is selected from isopropyl or cyclopentyl.

R⁵ is methyl.

Ring A, R¹, R² and q together form 1-acetyl-4-piperidinyl, 1-acetylpyrrolidin-3-yl, 1-benzyl-4-piperidinyl, 1-benzyloxycarbonyl-4-piperidinyl, 1-carbamoyl-4-piperidinyl, 1-ethoxycarbonyl-4-piperidinyl, 1-isopropyl-4-piperidinyl, 1-methyl-4-piperidinyl, 1-methylsulfonyl-4-piperidinyl, 1-methylsulfonylpyrrolidin-3-yl, 1-phenethyl-4-piperidinyl, 1-propyl-4-piperidinyl, 1-sulfamoyl-4-piperidinyl, 1-sulfamoylpyrrolidin-3-yl, 1-tert-butoxycarbonyl-4-piperidinyl, 1-tert-butoxycarbonylpyrrolidin-3-yl, 1-vinylsulfonyl-4-piperidinyl, (1R,5S)-3-(2-dimethylaminoethylsulfonyl)-3-azabicyclo[3.1.0]hex-6-yl, (1R,5S)-3-(2-pyrrolidin-1-ylethylsulfonyl)-3-azabicyclo[3.1.0]hex-6-yl, (1R,5S)-3-(3-dimethylaminopropylsulfonyl)-3-azabicyclo[3.1.0]hex-6-yl, (1R,5S)-3-(3-pyrrolidin-1-ylpropylsulfonyl)-3-azabicyclo[3.1.0]hex-6-yl, (1R,5S)-3-[2-(7-azabicyclo[2.2.1]hept-7-yl)ethylsulfonyl]-3-azabicyclo[3.1.0]hex-6-yl, (1R,5S)-3-[3-(1-piperidinyl)propylsulfonyl]-3-azabicyclo[3.1.0]hex-6-yl, (1R,5S)-3-[3-(2,5-dimethylpyrrolidin-1-yl)propylsulfonyl]-3-azabicyclo[3.1.0]hex-6-yl, (1R,5S)-3-[3-(7-azabicyclo[2.2.1]hept-7-yl)propylsulfonyl]-3-azabicyclo[3.1.0]hex-6-yl, (1R,5S)-3-[3-(cyclopentyl-methyl-amino)propylsulfonyl]-3-azabicyclo[3.1.0]hex-6-yl, (1R,5S)-3-azabicyclo[3.1.0]hex-6-yl, (1R,5S)-3-benzyloxycarbonyl-3-azabicyclo[3.1.0]hex-6-yl, (1R,5S)-3-methylsulfonyl-3-azabicyclo[3.1.0]hex-6-yl, (1R,5S)-8-methyl-8-azabicyclo[3.2.1]oct-3-yl, (3R)-1-methylsulfonyl-3-piperidinyl, (3R)-1-tert-butoxycarbonyl-3-piperidinyl, (3R)-3-piperidinyl, (3S)-1-methylsulfonyl-3-piperidinyl, (3S)-1-tert-butoxycarbonyl-3-piperidinyl, (3S)-3-piperidinyl, 1-(1-methylpiperidine-3-carbonyl)-4-piperidinyl, 1-(1-methylpyrrolidine-2-carbonyl)-4-piperidinyl, 1-(2-diethylaminoethylsulfamoyl)-4-piperidinyl, 1-(2-diethylaminoethylsulfonyl)-4-piperidinyl, 1-(2-dimethylaminoacetyl)-4-piperidinyl, 1-(2-dimethylaminoethylcarbamoyl)-4-piperidinyl, 1-(2-dimethylaminoethylcarbamoyl)pyrrolidin-3-yl, 1-(2-dimethylaminoethylsulfamoyl)-4-piperidinyl, 1-(2-dimethylaminoethylsulfonyl)-4-piperidinyl, 1-(2-methoxyethyl)-4-piperidinyl, 1-(2-morpholinoethylsulfamoyl)-4-piperidinyl, 1-(2-morpholinoethylsulfonyl)-4-piperidinyl, 1-(2-pyrrolidin-1-ylethylcarbamoyl)-4-piperidinyl, 1-(2-pyrrolidin-1-ylethylsulfamoyl)-4-piperidinyl, 1-(2-pyrrolidin-1-ylethylsulfonyl)-4-piperidinyl, 1-(2-thiomorpholinoethylsulfonyl)-4-piperidinyl, 1-(3-amino-3-methyl-butyl)sulfonyl-4-piperidinyl, 1-(3-chloropropylsulfonyl)-4-piperidinyl, 1-(3-chloropropylsulfonyl)pyrrolidin-3-yl, 1-(3-diethylaminopropylsulfonyl)-4-piperidinyl, 1-(3-dimethylamino-3-methyl-butyl)sulfonyl-4-piperidinyl, 1-(3-dimethylaminopropanoyl)-4-piperidinyl, 1-(3-dimethylaminopropyl-methyl-sulfamoyl)-4-piperidinyl, 1-(3-dimethylaminopropylsulfonyl)-4-piperidinyl, 1-(3-hydroxypropylsulfonyl)-4-piperidinyl, 1-(3-methyl-3-nitro-butyl)sulfonyl-4-piperidinyl, 1-(3-morpholinopropylsulfonyl)-4-piperidinyl, 1-(3-piperazin-1-ylpropanoyl)-4-piperidinyl, 1-(3-piperazin-1-ylpropanoyl)pyrrolidin-3-yl, 1-(3-pyrrolidin-1-ylpropylsulfamoyl)-4-piperidinyl, 1-(3-pyrrolidin-1-ylpropylsulfonyl)-4-piperidinyl, 1-(3-pyrrolidin-1-ylpropylsulfonyl)pyrrolidin-3-yl, 1-(4-dimethylaminobutanoyl)-4-piperidinyl, 1-(4-methyl-1,4-diazepane-1-carbonyl)-4-piperidinyl, 1-(4-methyl-1-tert-butoxycarbonyl-piperidine-4-carbonyl)-4-piperidinyl, 1-(4-methyl-1-tert-butoxy carbonyl-piperidine-4-carbonyl)pyrrolidin-3-yl, 1-(4-methylpiperazin-1-yl)sulfonyl-4-piperidinyl, 1-(4-methylpiperidine-4-carbonyl)-4-piperidinyl, 1-(4-methylpiperidine-4-carbonyl)pyrrolidin-3-yl, 1-(4-morpholinobutanoyl)-4-piperidinyl, 1-(4-piperidinylsulfonyl)-4-piperidinyl, 1-(4-tert-butoxycarbonylmorpholine-2-carbonyl)-4-piperidinyl, 1-(dimethylcarbamoyl)-4-piperidinyl, 1-(dimethylsulfamoyl)-4-piperidinyl, 1-(methylcarbamoyl)-4-piperidinyl, 1-(methylcarbamoyl)pyrrolidin-3-yl, 1-(morpholine-2-carbonyl)-4-piperidinyl, 1-[(1-benzyloxycarbonyl-3-piperidinyl)methylsulfonyl]-4-piperidinyl, 1-[(1-benzyloxycarbonyl-4-piperidinyl)sulfonyl]-4-piperidinyl, 1-[(1-isopropyl-4-piperidinyl)sulfamoyl]-4-piperidinyl, 1-[(1-isopropyl-4-piperidinyl)sulfonyl]-4-piperidinyl, 1-[(1-methyl-4-piperidinyl)carbamoyl]-4-piperidinyl, 1-[(1-methyl-4-piperidinyl)sulfamoyl]-4-piperidinyl, 1-[(1-methyl-4-piperidinyl)sulfonyl]-4-piperidinyl, 1-[(3-dimethylamino-2,2-dimethyl-propyl)sulfamoyl]-4-piperidinyl, 1-[(3R)-3-dimethylaminopyrrolidin-1-yl]sulfonyl-4-piperidinyl, 1-[(3S)-3-dimethylaminopyrrolidine-1-carbonyl]-4-piperidinyl, 1-[(4-methyl-1,4-diazepan-1-yl)sulfonyl]-4-piperidinyl, 1-[[(2R)-1-ethylpyrrolidin-2-yl]methylcarbamoyl]-4-piperidinyl, 1-[[(2S)-1-ethylpyrrolidin-2-yl]methylcarbamoyl]-4-piperidinyl, 1-[2-(1,4-oxazepan-4-yl)ethylsulfonyl]-4-piperidinyl, 1-[2-(1,4-thiazepan-4-yl)ethylsulfonyl]-4-piperidinyl, 1-[2-(1-methylpyrrolidin-2-yl)ethylsulfamoyl]-4-piperidinyl, 1-[2-(1-piperidinyl)ethylsulfonyl]-4-piperidinyl, 1-[2-(1-tert-butoxycarbonyl-4-piperidinyl)acetyl]-4-piperidinyl, 1-[2-(1-tert-butoxycarbonyl-4-piperidinyl)acetyl]pyrrolidin-3-yl, 1-[2-(2-methoxyethylamino)ethylsulfonyl]-4-piperidinyl, 1-[2-(3-oxopiperazin-1-yl)ethylsulfonyl]-4-piperidinyl, 1-[2-(4-acetyl-1,4-diazepan-1-yl)ethylsulfonyl]-4-piperidinyl, 1-[2-(4-acetylpiperazin-1-yl)ethylsulfonyl]-4-piperidinyl, 1-[2-(4-carbamoyl-1-piperidinyl)ethylsulfonyl]-4-piperidinyl, 1-[2-(4-cyano-1-piperidinyl)ethylsulfonyl]-4-piperidinyl, 1-[2-(4-fluoro-1-piperidinyl)ethylsulfonyl]-4-piperidinyl, 1-[2-(4-methyl-1-piperidinyl)ethylsulfonyl]-4-piperidinyl, 1-[2-(4-methylpiperazin-1-yl)ethylsulfamoyl]-4-piperidinyl, 1-[2-(4-methylpiperazin-1-yl)ethylsulfonyl]-4-piperidinyl, 1-[2-(4-piperidinyl)acetyl]-4-piperidinyl, 1-[2-(4-piperidinyl)acetyl]pyrrolidin-3-yl, 1-[2-(4-propyl-1-piperidinyl)ethylsulfonyl]-4-piperidinyl, 1-[2-(5-oxo-1,4-diazepan-1-yl)ethylsulfonyl]-4-piperidinyl, 1-[2-(7-azabicyclo[2.2.1]hept-7-yl)ethylsulfonyl]-4-piperidinyl, 1-[2-(8-azabicyclo[2.2.2]oct-8-yl)ethylsulfonyl]-4-piperidinyl, 1-[2-(azetidin-1-yl)ethylsulfonyl]-4-piperidinyl, 1-[2-(cyclopropyl-methyl-amino)ethylsulfonyl]-4-piperidinyl, 1-[2-(cyclopropylmethyl-methyl-amino)ethylsulfonyl]-4-piperidinyl, 1-[2-(isopropyl-methyl-amino)ethylsulfonyl]-4-piperidinyl, 1-[2-[(3R)-1-tert-butoxycarbonyl-3-piperidinyl]acetyl]-4-piperidinyl, 1-[2-[(3R)-1-tert-butoxycarbonyl-3-piperidinyl]acetyl]pyrrolidin-3-yl, 1-[2-[(3R)-3-fluoropyrrolidin-1-yl]ethylsulfonyl]-4-piperidinyl, 1-[2-[(3R)-3-piperidinyl]acetyl]-4-piperidinyl, 1-[2-[(3R)-3-piperidinyl]acetyl]pyrrolidin-3-yl, 1-[2-[(3S)-1-tert-butoxycarbonyl-3-piperidinyl]acetyl]-4-piperidinyl, 1-[2-[(3S)-1-tert-butoxycarbonyl-3-piperidinyl]acetyl]pyrrolidin-3-yl, 1-[2-[(3S)-3-fluoropyrrolidin-1-yl]ethylsulfonyl]-4-piperidinyl, 1-[2-[(3S)-3-piperidinyl]acetyl]-4-piperidinyl, 1-[2-[(3S)-3-piperidinyl]acetyl]pyrrolidin-3-yl, 1-[3-(1-piperidinyl)propylsulfonyl]-4-piperidinyl, 1-[3-(1-piperidinyl)propylsulfonyl]-4-piperidinyl, 1-[3-(1-tert-butoxycarbonyl-4-piperidinyl)propanoyl]-4-piperidinyl, 1-[3-(1-tert-butoxycarbonyl-4-piperidinyl)propanoyl]pyrrolidin-3-yl, 1-[3-(2-cyanoethyl-ethyl-amino)propylsulfonyl]-4-piperidinyl, 1-[3-(2-cyanoethyl-methyl-amino)propylsulfonyl]-4-piperidinyl, 1-[3-(2-hydroxypropylamino)propylsulfonyl]-4-piperidinyl, 1-[3-(2-methoxyethylamino)propylsulfonyl]-4-piperidinyl, 1-[3-(2-methoxyethylamino)propylsulfonyl]pyrrolidin-3-yl, 1-[3-(2-methoxyethyl-methyl-amino)propylsulfonyl]-4-piperidinyl, 1-[3-(4-isopropylpiperazin-1-yl)propylsulfonyl]pyrrolidin-3-yl, 1-[3-(4-methylpiperazin-1-yl)propylsulfonyl]-4-piperidinyl, 1-[3-(4-methylpiperazin-1-yl)propylsulfonyl]pyrrolidin-3-yl, 1-[3-(4-piperidinyl)propanoyl]-4-piperidinyl, 1-[3-(4-piperidinyl)propanoyl]pyrrolidin-3-yl, 1-[3-(4-tert-butoxycarbonylpiperazin-1-yl)propanoyl]-4-piperidinyl, 1-[3-(4-tert-butoxycarbonylpiperazin-1-yl)propanoyl]pyrrolidin-3-yl, 1-[3-(7-azabicyclo[2.2.1]hept-7-yl)propylsulfonyl]-4-piperidinyl, 1-[3-(8-azabicyclo[2.2.2]oct-8-yl)propylsulfonyl]-4-piperidinyl, 1-[3-(azetidin-1-yl)propylsulfonyl]-4-piperidinyl, 1-[3-(cyclobutylamino)propylsulfonyl]-4-piperidinyl, 1-[3-(cyclobutyl-methyl-amino)propylsulfonyl]-4-piperidinyl, 1-[3-(cyclopentylamino)propylsulfonyl]-4-piperidinyl, 1-[3-(cyclopentyl-methyl-amino)propylsulfonyl]-4-piperidinyl, 1-[3-(cyclopropylamino)propylsulfonyl]-4-piperidinyl, 1-[3-(cyclopropylmethylamino)propylsulfonyl]-4-piperidinyl, 1-[3-(cyclopropyl-methyl-amino)propylsulfonyl]-4-piperidinyl, 1-[3-(cyclopropylmethyl-methyl-amino)propylsulfonyl]-4-piperidinyl, 1-[3-(ethyl-methyl-amino)propylsulfonyl]-4-piperidinyl, 1-[3-(isopropylamino)propylsulfonyl]-4-piperidinyl, 1-[3-(isopropyl-methyl-amino)propylsulfonyl]-4-piperidinyl, 1-[3-[(2-hydroxy-1,1-dimethyl-ethyl)amino]propylsulfonyl]-4-piperidinyl, 1-[3-[(2-hydroxy-1-methyl-ethyl)amino]propylsulfonyl]-4-piperidinyl, 1-[3-[(2-methoxy-1-methyl-ethyl)amino]propylsulfonyl]-4-piperidinyl, 1-[3-[(3S)-3-fluoropyrrolidin-1-yl]propylsulfamoyl]-4-piperidinyl, 1-[3-[[1-(hydroxymethyl)-2-methyl-propyl]amino]propylsulfonyl]-4-piperidinyl, 1-[3-[1-(hydroxymethyl)propylamino]propylsulfonyl]-4-piperidinyl, 1-[3-[4-(2-hydroxyethyl)piperazin-1-yl]propylsulfonyl]-4-piperidinyl, 4-piperidinyl, azepan-3-yl, pyrrolidin-3-yl and 1-(tert-butoxycarbonyl)azepan-3-yl.

Therefore in a further aspect of the invention there is provided a compound of formula (I) (as depicted above) wherein:

Ring A is a 5 or 6 membered saturated heterocyclic ring which contains one nitrogen atom; wherein 2 atoms of Ring A may optionally be connected by a bridge;

R¹ is a substituent on nitrogen and is selected from hydrogen, C₁₋₆alkyl, C₁₋₆alkanoyl, sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸;

q is 0;

n is 0;

R⁴ is isopropyl;

R⁵ is methyl;

R⁷ is —C(O)—;

R⁸ is selected from halo, hydroxy, C₁₋₆alkyl, C₁₋₆alkoxy, N—(C₁₋₆alkyl)amino, carbocyclyl-R¹⁷— or heterocyclyl-R¹⁸—; wherein R⁸ may be optionally substituted on carbon by one or more R¹⁹; and wherein if said heterocyclyl contains an —NH— moiety that nitrogen may be optionally substituted by a group selected from R²⁰;

R²⁰ is selected from C₁₋₆alkyl or C₁₋₆alkoxycarbonyl; wherein R²⁰ may be optionally substituted on carbon by one or more R²¹;

R¹⁷ and R¹⁸ are a direct bond; and

R¹⁹ and R²¹ are independently selected from hydroxy and methoxy; or a pharmaceutically acceptable salt or an in vivo hydrolysable ester thereof.

Therefore in a further aspect of the invention there is provided a compound of formula (I) (as depicted above) wherein:

Ring A is a 5 or 6 membered saturated heterocyclic ring which contains one nitrogen atom; wherein 2 atoms of Ring A may optionally be connected by a bridge;

R¹ is a substituent on nitrogen and is selected from hydrogen, C₁₋₆alkyl, C₁₋₆alkanoyl, carbamoyl, N—(C₁₋₆alkyl)carbamoyl, N,N—(C₁₋₆alkyl)carbamoyl, sulphamoyl, N—(C₁₋₆alkyl)sulphamoyl, N,N—(C₁₋₆allyl)₂sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;

q is 0;

R³ is halo;

n is 0 or 1;

R⁴ is selected from isopropyl or cyclopentyl;

R⁵ is methyl;

R⁷ is selected from —C(O)—, —C(O)N—(R¹⁵)—, —S(O)₂— or —SO₂N—(R¹⁶)—; wherein R¹⁵ and R¹⁶ are hydrogen;

R⁸ is selected from halo, nitro, hydroxy, amino, C₁₋₆alkyl, C₁₋₆alkoxy, N,N—(C₁₋₆allyl)amino, N,N—(C₁₋₆alkyl)₂amino, carbocyclyl-R¹⁷— or heterocyclyl-R¹⁸—; wherein R⁸may be optionally substituted on carbon by one or more R¹⁹; and wherein if said heterocyclyl contains an —NH— moiety that nitrogen may be optionally substituted by a group selected from R²⁰;

R⁹ and R²⁰ are independently selected from C₁₋₆alkyl, C₁₋₆alkanoyl, C₁₋₆alkoxycarbonyl and benzyloxycarbonyl; wherein R⁹ and R²⁰ independently of each other, may be optionally substituted on carbon by one or more R²¹;

R¹⁷ and R¹⁸ are independently selected from a direct bond or —N—(R²²)—; wherein R²² is selected from hydrogen or C₁₋₆alkyl; and

R¹⁹ and R²¹ are independently selected from halo, cyano, hydroxy, carbamoyl, methyl, propyl, cyclopropyl and methoxy;

or a pharmaceutically acceptable salt or an in vivo hydrolysable ester thereof.

Therefore in a further aspect of the invention there is provided a compound of formula (I) (as depicted above) wherein:

Ring A is (1α, 5α, 6α)-3-azabicyclo[3.1.0]hexan-3-yl, (R)-piperidin-3-yl, (S)-piperidin-3-yl, piperidin-4-yl, pyrrolidin-3-yl or endo-8-azabicyclo[3.2.1]octan-3-yl;

R¹ is a substituent on nitrogen and is selected from hydrogen, methyl, propyl, isopropyl, ethenylsulphonyl, mesyl, benzyloxycarbonyl, f-butoxycarbonyl, acetyl, phenethyl, ethoxycarbonyl, 2-methoxyethyl, sulphamoyl, N,N-dimethylsulphamoyl, N,N-dimethylcarbamoyl, benzyl, carbamoyl, N-methylcarbamoyl, 2-(dimethylamino)ethylsulphonyl, 2-(N-methyl-N-isopropyl)ethylsulphonyl, 2-(1-methylpiperazin-4-yl)ethylsulphonyl, 2-pyrrolidin-1-ylethylsulphonyl, 2-(3-fluoropyrrolidin-1-yl)ethylsulphonyl, 2-(thiomorpholin-4-yl)ethylsulphonyl, 2-(4-methylpiperidin-1-yl)ethylsulphonyl, 2-(homopiperidin-1-yl)ethylsulphonyl, 2-diethylaminoethylsulphonyl, 2-azetidin-1-ylethylsulphonyl, 2-morpholinoethylsulphonyl, 2-(4-fluoropiperidin-1-ylethylsulphonyl, 2-(4-cyanopiperidin-1-ylethylsulphonyl, 2-(4-propylpiperidin-1-ylethylsulphonyl, 2-(4-carbamoylpiperidin-1-yl)ethylsulphonyl, 2-(7-azabicyclo[2.2.1]hept-7-yl)ethylsulphonyl, 2-(2-azabicyclo[2.2.2]oct-2-yl)ethylsulfonyl, 2-(6-azabicyclo[2.2.2]oct-6-yl)ethylsulfonyl, 2-homomorpholinoethylsulphonyl, 2-(2-oxopiperazin-4-yl)ethylsulphonyl, 2-(1-acetylpiperazin-4-yl)ethylsulphonyl, 2-(2-methoxyethylamino)ethylsulphonyl, 2-(N-methyl-N-cyclopropyl)ethylsulphonyl, 2-(2-oxohomopiperazin-4-yl)ethylsulphonyl, 2-(1-acetylhomopiperazin-4-yl)ethylsulphonyl, 2-(N-methyl-N-cyclopropylmethyl)ethylsulphonyl, 2-(homothiomorpholin-4-yl)ethylsulphonyl, 3-chloropropylsulphonyl, 3-dimethylaminopropylsulphonyl, 3-dimethylamino-2,2-dimethylpropylsulphonyl, 3-diethylaminopropylsulphonyl, 3-(2-methoxyethylamino)propylsulphonyl, 3-[N-methyl-N-(2-methoxyethyl)amino]propylsulphonyl, 3-hydroxypropylsulphonyl, 3-(1-hydroxyprop-2-ylamino)propylsulphonyl, 3-(1-methylpiperazin-4-yl)propylsulphonyl, 3-(1-isopropylpiperazin-4-yl)propylsulphonyl, 3-(6-azabicyclo[2.2.2]oct-6-yl)propylsulfonyl, 3-(7-azabicyclo[2.2.1]hept-7-yl)propylsulphonyl, 3-[1-(2-hydroxyethyl)piperazin-4-yl]propylsulphonyl, 3-pyrrolidin-1-ylpropylsulphonyl, 3-(1,4-dimethylpyrrolidin-1-yl)propylsulphonyl, 3-morpholinopropylsulphonyl, 3-(1-hydroxybut-2-ylamino)propylsulphonyl, 3-(1-methoxyprop-2-ylamino)propylsulphonyl, 3-(2-hydroxypropylamino)propylsulphonyl, 3-(1-hydroxy-3-methylbut-2-ylamino)propylsulphonyl, 3-(1-hydroxy-2-methylprop-2-ylamino)propylsulphonyl, 3-(piperidin-1-yl)propylsulphonyl, 3-(cyclopropylamino)propylsulphonyl, 3-(N-methyl-N-cyclopropylamino)propylsulphonyl, 3-(cyclopentylamino)propylsulphonyl, 3-(N-methyl-N-cyclopentylamino)propylsulphonyl, 3-(cyclobutylamino)propylsulphonyl, 3-(N-methyl-N-cyclobutylamino)propylsulphonyl, 3-(isopropylamino)propylsulphonyl, 3-(N-methyl-N-isopropylamino)propylsulphonyl, 3-(N-methyl-N-ethylamino)propylsulphonyl, 3-[N-methyl-N-(2-cyanoethyl)amino]propylsulphonyl, 3-[N-ethyl-N-(2-cyanoethyl)amino]propylsulphonyl, 3-azetidin-1-ylpropylsulphonyl, 3-(cyclopropylmethylamino)propylsulphonyl, 3-(N-methyl-N-cyclopropylmethylamino)propylsulphonyl, 3-nitro-3-methylbutylsulphonyl, 3-amino-3-methylbutylsulphonyl, 3-dimethyl-3-methylbutylsulphonyl, 2-(piperidin-3-yl)acetyl, 2-(1-t-butoxycarbonylpiperidin-3-yl)acetyl, 2-(piperidin-4-yl)acetyl, 2-(1-t-butoxycarbonylpiperidin-4-yl)acetyl, 2-dimethylaminoacetyl, 3-(1-t-butoxycarbonylpiperazin-4-yl)propanoyl, 3-(1-t-butoxycarbonylpiperidin-4-yl)propanoyl, 3-(piperidin-4-yl)propanoyl, 3-(piperazin-4-yl)propanoyl, 3-dimethylaminopropanoyl, 4-morpholinobutanoyl, 4-dimethylaminobutanoyl, 1-t-butoxycarbonyl-4-methylpiperidin-4-ylcarbonyl, 4-methylpiperidin-4-ylcarbonyl, 1-t-butoxycarbonyl-4-methylpiperidin-4-ylcarbonyl, 4-methylhomopiperazin-1-ylcarbonyl, 1-methylpiperidin-3-ylcarbonyl, 1-methylpyrrolidin-2-ylcarbonyl, 3-dimethylaminopyrrolidin-1-ylcarbonyl, 4−/−butoxycarbonylmorpholin-2-ylcarbonyl, morpholin-2-ylcarbonyl, 1-methylpiperidin-4-ylcarbamoyl, N-(1-ethylpyrrolidin-2-ylmethyl)carbamoyl, N-(2-pyrrolidin-1-ylethyl)carbamoyl, N-(2-dimethylaminoethyl)carbamoyl, N-(1-methylpiperidin-4-yl)sulphamoyl, N-(1-isopropylpiperidin-4-yl)sulphamoyl, 2-(dimethylamino)ethylsulphamoyl, 2-(diethylamino)ethylsulphamoyl, 2-(morpholino)ethylsulphamoyl, 2-(1-methylpiperazin-4-yl)ethylsulphamoyl, 2-(1-methylpyrrolidin-2-yl)ethylsulphamoyl, 3-(pyrrolidin-1-yl)propylsulphamoyl, 3-(3-fluoropyrrolidin-1-yl)propylsulphamoyl, 3-dimethylamino-2,2-dimethylpropylsulphamoyl, 3-(piperidin-1-yl)propylsulphamoyl, N-methyl-N-(3-dimethylaminopropyl)sulphamoyl, 3-dimethylaminopyrrolidin-1-ylsulphonyl, 1-methylpiperazin-4-ylsulphonyl, 1-methylpiperidin-4-ylsulphonyl, 1-isopropylpiperidin-4-ylsulphonyl and 1-methylhomopiperazin-4-ylsulphonyl;

q is 0;

R³ is fluoro or chloro;

n is 0 or 1;

R⁴ is selected from isopropyl or cyclopentyl;

R⁵ is methyl;

or a pharmaceutically acceptable salt or an in vivo hydrolysable ester thereof.

Therefore in a further aspect of the invention there is provided a compound of formula (I) (as depicted above) wherein:

Ring A is a 5-7 membered saturated heterocyclic ring which contains one nitrogen atom; wherein 2 atoms of Ring A may optionally be connected by a bridge;

R¹ is a substituent on nitrogen and is selected from hydrogen, C₁₋₆alkyl, C₁₋₆alkanoyl, carbamoyl, N—(C₁₋₆alkylcarbamoyl, N,N—(C₁₋₆alkyl)carbamoyl, sulphamoyl, N—(C₁₋₆alkyl)sulphamoyl, N,N—(C₁₋₆alkyl)₂sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;

q is 0;

R³ is halo;

n is 0 or 1;

R⁴ is selected from isopropyl or cyclopentyl;

R⁵ is methyl;

R⁷ is selected from —C(O)—, —C(O)N—(R¹⁵)—, —S(O)₂— or —SO₂N—(R¹⁶)—; wherein R¹⁵ and R¹⁶ are hydrogen;

R⁸ is selected from halo, nitro, hydroxy, amino, C N,N-alkyl, C₁₋₆alkoxy, N—(C₁₋₆alkyl)amino, N,N—(C₁₋₆alkyl)₂amino, carbocyclyl-R¹⁷— or heterocyclyl-R¹⁸—; wherein R⁸may be optionally substituted on carbon by one or more R¹⁹; and wherein if said heterocyclyl contains an —NH— moiety that nitrogen may be optionally substituted by a group selected from R²⁰;

R⁹ and R²⁰ are independently selected from C₁₋₆alkyl, C₁₋₆alkanoyl, C₁₋₆alkoxycarbonyl and benzyloxycarbonyl; wherein R⁹ and R²⁰ independently of each other, may be optionally substituted on carbon by one or more R²¹;

R¹⁷ and R¹⁸ are independently selected from a direct bond or —N—(R²²)—; wherein R²² is selected from hydrogen or C₁₋₆alkyl; and

R¹⁹ and R²¹ are independently selected from halo, cyano, hydroxy, carbamoyl, methyl, propyl, cyclopropyl and methoxy;

or a pharmaceutically acceptable salt or an in vivo hydrolysable ester thereof.

Therefore in a further aspect of the invention there is provided a compound of formula (I) (as depicted above) wherein:

Ring A is azepan-3-yl, (1α, 5α, 6α)-3-azabicyclo[3.1.0]hexan-3-yl, (R)-piperidin-3-yl, (S)-piperidin-3-yl, piperidin-4-yl, pyrrolidin-3-yl or endo-8-azabicyclo[3.2.1]octan-3-yl;

R¹ is a substituent on nitrogen and is selected from hydrogen, methyl, propyl, isopropyl, ethenylsulphonyl, mesyl, benzyloxycarbonyl, t-butoxycarbonyl, acetyl, phenethyl, ethoxycarbonyl, 2-methoxyethyl, sulphamoyl, N,N-dimethylsulphamoyl, N,N-dimethylcarbamoyl, benzyl, carbamoyl, N-methylcarbamoyl, 2-(dimethylamino)ethylsulphonyl, 2-(N-methyl-N-isopropyl)ethylsulphonyl, 2-(1-methylpiperazin-4-yl)ethylsulphonyl, 2-pyrrolidin-1-ylethylsulphonyl, 2-(3-fluoropyrrolidin-1-yl)ethylsulphonyl, 2-(thiomorpholin-4-yl)ethylsulphonyl, 2-(4-methylpiperidin-1-yl)ethylsulphonyl, 2-(homopiperidin-1-yl)ethylsulphonyl, 2-diethylaminoethylsulphonyl, 2-azetidin-1-ylethylsulphonyl, 2-morpholinoethylsulphonyl, 2-(4-fluoropiperidin-1-ylethylsulphonyl, 2-(4-cyanopiperidin-1-ylethylsulphonyl, 2-(4-propylpiperidin-1-ylethylsulphonyl, 2-(4-carbamoylpiperidin-1-ylethylsulphonyl, 2-(7-azabicyclo[2.2.1]hept-7-yl)ethylsulphonyl, 2-(2-azabicyclo[2.2.2]oct-2-yl)ethylsulfonyl, 2-(6-azabicyclo[2.2.2]oct-6-yl)ethylsulfonyl, 2-homomorpholinoethylsulphonyl, 2-(2-oxopiperazin-4-yl)ethylsulphonyl, 2-(1-acetylpiperazin-4-yl)ethylsulphonyl, 2-(2-methoxyethylamino)ethylsulphonyl, 2-(N-methyl-N-cyclopropyl)ethylsulphonyl, 2-(2-oxohomopiperazin-4-yl)ethylsulphonyl, 2-(1-acetylhomopiperazin-4-yl)ethylsulphonyl, 2-(N-methyl-N-cyclopropylmethyl)ethylsulphonyl, 2-(homothiomorpholin-4-yl)ethylsulphonyl, 3-chloropropylsulphonyl, 3-dimethylaminopropylsulphonyl, 3-dimethylamino-2,2-dimethylpropylsulphonyl, 3-diethylaminopropylsulphonyl, 3-(2-methoxyethylamino)propylsulphonyl, 3-[N-methyl-N-(2-methoxyethyl)amino]propylsulphonyl, 3-hydroxypropylsulphonyl, 3-(1-hydroxyprop-2-ylamino)propylsulphonyl, 3-(1-methylpiperazin-4-yl)propylsulphonyl, 3-(1-isopropylpiperazin-4-yl)propylsulphonyl, 3-(6-azabicyclo[2.2.2]oct-6-yl)propylsulfonyl, 3-(7-azabicyclo[2.2.1]hept-7-yl)propylsulphonyl, 3-[1-(2-hydroxyethyl)piperazin-4-yl]propylsulphonyl, 3-pyrrolidin-1-ylpropylsulphonyl, 3-(1,4-dimethylpyrrolidin-1-yl)propylsulphonyl, 3-morpholinopropylsulphonyl, 3-(1-hydroxybut-2-ylamino)propylsulphonyl, 3-(1-methoxyprop-2-ylamino)propylsulphonyl, 3-(2-hydroxypropylamino)propylsulphonyl, 3-(1-hydroxy-3-methylbut-2-ylamino)propylsulphonyl, 3-(1-hydroxy-2-methylprop-2-ylamino)propylsulphonyl, 3-(piperidin-1-yl)propylsulphonyl, 3-(cyclopropylamino)propylsulphonyl, 3-(N-methyl-N-cyclopropylamino)propylsulphonyl, 3-(cyclopentylamino)propylsulphonyl, 3-(N-methyl-N-cyclopentylamino)propylsulphonyl, 3-(cyclobutylamino)propylsulphonyl, 3-(N-methyl-N-cyclobutylamino)propylsulphonyl, 3-(isopropylamino)propylsulphonyl, 3-(N-methyl-N-isopropylamino)propylsulphonyl, 3-(N-methyl-N-ethylamino)propylsulphonyl, 3-[N-methyl-N-(2-cyanoethyl)amino]propylsulphonyl, 3-[N-ethyl-N-(2-cyanoethyl)amino]propylsulphonyl, 3-azetidin-1-ylpropylsulphonyl, 3-(cyclopropylmethylamino)propylsulphonyl, 3-(N-methyl-N-cyclopropylmethylamino)propylsulphonyl, 3-nitro-3-methylbutylsulphonyl, 3-amino-3-methylbutylsulphonyl, 3-dimethyl-3-methylbutylsulphonyl, 2-(piperidin-3-yl)acetyl, 2-(1-t-butoxycarbonylpiperidin-3-yl)acetyl, 2-(piperidin-4-yl)acetyl, 2-(1-t-butoxycarbonylpiperidin-4-yl)acetyl, 2-dimethylaminoacetyl, 3-(1-t-butoxycarbonylpiperazin-4-yl)propanoyl, 3-(1-t-butoxycarbonylpiperidin-4-yl)propanoyl, 3-(piperidin-4-yl)propanoyl, 3-(piperazin-4-yl)propanoyl, 3-dimethylaminopropanoyl, 4-morpholinobutanoyl, 4-dimethylaminobutanoyl, 1-t-butoxycarbonyl-4-methylpiperidin-4-ylcarbonyl, 4-methylpiperidin-4-ylcarbonyl, 1-t-butoxycarbonyl-4-methylpiperidin-4-ylcarbonyl, 4-methylhomopiperazin-1-ylcarbonyl, 1-methylpiperidin-3-ylcarbonyl, 1-methylpyrrolidin-2-ylcarbonyl, 3-dimethylaminopyrrolidin-1-ylcarbonyl, 4−/−butoxycarbonylmorpholin-2-ylcarbonyl, morpholin-2-ylcarbonyl, 1-methylpiperidin-4-ylcarbamoyl, N-(1-ethylpyrrolidin-2ylmethyl)carbamoyl, N-(2-pyrrolidin-1-ylethyl)carbamoyl, N-(2-dimethylaminoethyl)carbamoyl, 2-(1-methylpiperidin-4-yl)sulphamoyl, N-(1-isopropylpiperidin-4-yl)sulphamoyl, 2-(dimethylamino)ethylsulphamoyl, 2-(diethylamino)ethylsulphamoyl, 2-(morpholino)ethylsulphamoyl, 2-(1-methylpiperazin-4-yl)ethylsulphamoyl, 2-(1-methylpyrrolidin-2-yl)ethylsulphamoyl, 3-(pyrrolidin-1-yl)propylsulphamoyl, 3-(3-fluoropyrrolidin-1-yl)propylsulphamoyl, 3-dimethylamino-2,2-dimethylpropylsulphamoyl, 3-(piperidin-1-yl)propylsulphamoyl, N-methyl-N-(3-dimethylaminopropyl)sulphamoyl, 3-dimethylaminopyrrolidin-1-ylsulphonyl, 1-methylpiperazin-4-ylsulphonyl, 1-methylpiperidin-4-ylsulphonyl, 1-isopropylpiperidin-4-ylsulphonyl and 1-methylhomopiperazin-4-ylsulphonyl;

q is 0;

R³ is fluoro or chloro;

n is 0 or 1;

R⁴ is selected from isopropyl or cyclopentyl;

R⁵ is methyl;

or a pharmaceutically acceptable salt or an in vivo hydrolysable ester thereof.

In another aspect of the invention, preferred compounds of the invention are any one of the Examples or a pharmaceutically acceptable salt or an in vivo hydrolysable ester thereof.

In a further aspect of the invention, a particular compound is any one of Examples 129, 138, 140, 142, 144, 145, 146, 149, 150 or 187 or a pharmaceutically acceptable salt or an in vivo hydrolysable ester thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or an in vivo hydrolysable ester thereof which process (wherein variable groups are, unless otherwise specified, as defined in formula (I)) comprises of:

Process a) reaction of a pyrimidine of formula (II):

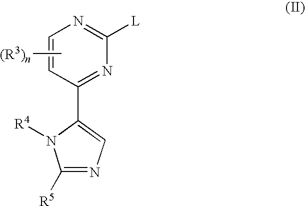

wherein L is a displaceable group; with an amine of formula (III):

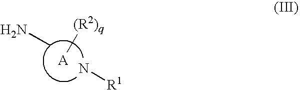

or
Process b) reacting a compound of formula (IV):

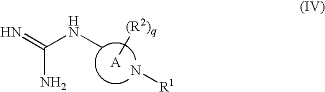

with a compound of formula (V):

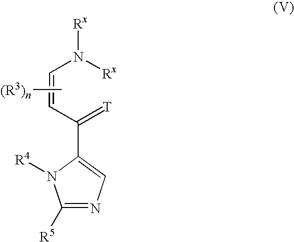

wherein T is O or S; R^{x} may be the same or different and is selected from C₁₋₆alkyl; or
Process c) reacting a pyrimidine of formula (VI):

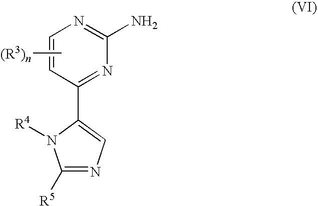

with a compound of formula (VII):

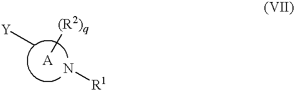

where Y is a displaceable group;
and thereafter if necessary:
i) converting a compound of the formula (I) into another compound of the formula (I);
ii) removing any protecting groups;
iii) forming a pharmaceutically acceptable salt or in vivo hydrolysable ester.

L is a displaceable group, suitable values for L are for example, a halogeno or sulphonyloxy group, for example a chloro, bromo, methanesulphonyloxy or toluene-4-sulphonyloxy group.

Y is a displaceable group, suitable values for Y are for example, a halogeno or sulphonyloxy group, for example a bromo, iodo or trifluoromethanesulphonyloxy group. Preferably Y is iodo.

Specific reaction conditions for the above reactions are as follows.

Process a) Pyrimidines of formula (II) and amines of formula (III) may be reacted together in a suitable solvent such as tetrahydrofuran, N-methylpyrrolidinone or isopropyl alcohol, or can be reacted together neat, at a temperature in the range of 25-200° C., particularly in the range of 60-160° C. The reaction may be conducted in the presence of a suitable base such as, for example, N,N-diisopropylethylamine, sodium hydride or potassium carbonate.

Pyrimidines of the formula (II) where L is chloro may be prepared according to Scheme 1:

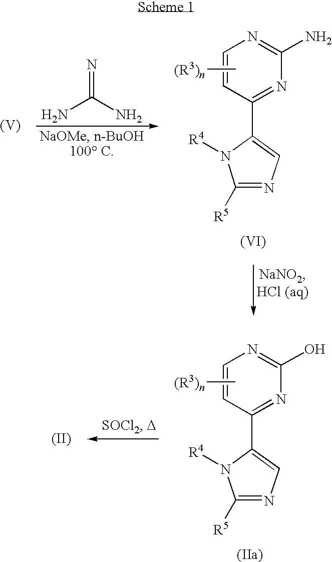

Amines of formula (III) are commercially available compounds, or they are known in the literature, or they are prepared by standard processes known in the art.

Process b) Compounds of formula (IV) and compounds of formula (V) are reacted together in a suitable solvent such as N-methylpyrrolidinone or butanol at a temperature in the range of 100-200° C., preferably in the range of 150-170° C. The reaction is preferably conducted in the presence of a suitable base such as, for example, sodium hydride, sodium methoxide or potassium carbonate.

Compounds of formula (V) may be prepared according to Scheme 2:

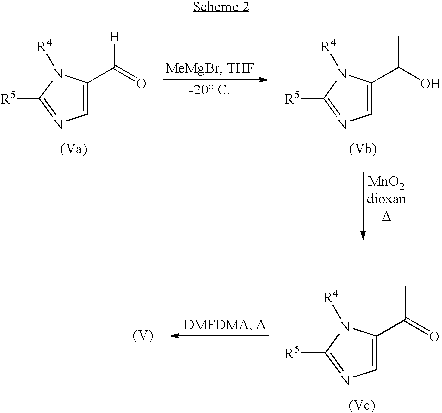

Compounds of formula (IV) and (Va) are commercially available compounds, or they are known in the literature, or they are prepared by standard processes known in the art.

Process c) Compounds of formula (VI) and amines of formula (VII) may be reacted together under the conditions described in Process a.

The synthesis of compounds of formula (VI) is described in Scheme 1.

Compounds of formula (VII) are commercially available compounds, or they are known in the literature, or they are prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a 3 methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cell-proliferation activity such as anti-cancer activity which is believed to arise from the CDK inhibitory activity of the compound. These properties may be assessed, for example, using the procedure set out below:—

### Assay

The following abbreviations have been used:—

HEPES is N-[2-Hydroxyethyl]piperazine-N′-[2-ethanesulfonic acid]

### DTT is Dithiothreitol

PMSF is Phenylmethylsulphonyl fluoride

The compounds were tested in an in vitro kinase assay in 96 well format using Scintillation Proximity Assay (SPA—obtained from Amersham) for measuring incorporation of [γ-33-P]-Adenosine Triphosphate into a test substrate (GST-Retinoblastoma protein; GST-Rb). In each well was placed the compound to be tested (diluted in DMSO and water to correct concentrations) and in control wells either roscovitine as an inhibitor control or DMSO as a positive control.

Approximately 0.2 μl of CDK2/Cyclin E partially-purified enzyme (amount dependent on enzyme activity) diluted in 25 μl incubation buffer was added to each well then 20 μl of GST-Rb/ATP/ATP33 mixture (containing 0.5 μg GST-Rb and 0.2 μM ATP and 0.14 μCi [γ-33-P]-Adenosine Triphosphate in incubation buffer), and the resulting mixture shaken gently, then incubated at ambient temperature for 60 mins.

To each well was then added 150 μl stop solution containing (0.8 mg/well of Protein A-PVT SPA bead (Amersham)), 20 pM/well of Anti-Glutathione Transferase, Rabbit IgG (obtained from Molecular Probes), 61 mM EDTA and 50 mM HEPES pH 7.5 containing 0.05% sodium azide.

The plates were sealed with Topseal-S plate sealers, left for two hrs then spun at 2500 rpm, 1124×g., for 5 mins. The plates were read on a Topcount for 30 seconds per well.

The incubation buffer used to dilute the enzyme and substrate mixes contained 50 mM HEPES pH7.5, 10 mM MnCl₂, 1 mM DTT, 100 μM Sodium vanadate, 100 μM NaF, 10 mM Sodium Glycerophosphate, BSA (1 mg/ml final).

### Test Substrate

In this assay only part of the retinoblastoma protein (Science 1987 Mar. 13; 235 (4794):1394-1399; Lee W. H., Bookstein R., Hong F., Young L. J., Shew J. Y., Lee E. Y.) was used, fused to a GST tag. PCR of retinoblastoma gene encoding amino acids 379-928 (obtained from retinoblastoma plasmid ATCC pLRbRNL) was performed, and the sequence cloned into pGEx 2 T fusion vector (Smith D. B. and Johnson, K. S. Gene 67, 31 (1988); which contained a tac promoter for inducible expression, internal lac I^{q} gene for use in any E. Coli host, and a coding region for thrombin cleavage—obtained from Pharmacia Biotech) which was used to amplify amino acids 792-928. This sequence was again cloned into pGEx 2 T.

The retinoblastoma 792-928 sequence so obtained was expressed in E. Coli (BL21 (DE3) pLysS cells) using standard inducible expression techniques, and purified as follows.

E. coli paste was resuspended in 10 ml/g of NETN buffer (50 mM Tris pH 7.5, 120 mM NaCl, 1 mM EDTA, 0.5% v/v NP-40, 1 mM PMSF, 1 ug/ml leupeptin, 1 ug/ml aprotinin and 1 ug/ml pepstatin) and sonicated for 2×45 seconds per 100 ml homogenate. After centrifugation, the supernatant was loaded onto a 10 ml glutathione Sepharose column (Pharmacia Biotech, Herts, UK), and washed with NETN buffer. After washing with kinase buffer (50 mM HEPES pH 7.5, 10 mM MgCl2, 1 mM DTT, 1 mM PMSF, 1 ug/ml leupeptin, 1 ug/ml aprotinin and 1 ug/ml pepstatin) the protein was eluted with 50 mM reduced glutathione in kinase buffer. Fractions containing GST-Rb(792-927) were pooled and dialysed overnight against kinase buffer. The final product was analysed by Sodium Dodeca Sulfate (SDS) PAGE (Polyacrylamide gel) using 8-16% Tris-Glycine gels (Novex, San Diego, USA).

### CDK2 and Cyclin E

The open reading frames of CDK2 and Cyclin E were isolated by reverse transcriptase-PCR using HeLa cell and activated T cell mRNA as a template and cloned into the insect expression vector pVL1393 (obtained from Invitrogen 1995 catalogue number: V1392-20). CDK2 and cyclin E were then dually expressed [using a standard virus Baculogold co-infection technique] in the insect SF21 cell system (Spodoptera Frugiperda cells derived from ovarian tissue of the Fall Army Worm—commercially available).

### Example Production of Cyclin E/CDK2

The following Example provides details of the production of Cyclin E/CDK2 in SF21 cells (in TC100+10% FBS(TCS)+0.2% Pluronic) having dual infection MOI 3 for each virus of Cyclin E & CDK2.

SF21 cells grown in a roller bottle culture to 2.33×10⁶cells/ml were used to inoculate 10×500 ml roller bottles at 0.2×10E6 cells/ml. The roller bottles were incubated on a roller rig at 28° C.

After 3 days (72 hrs.) the cells were counted, and the average from 2 bottles found to be 1.86×10E6 cells/ml. (99% viable). The cultures were then infected with the dual viruses at an MOI 3 for each virus.

The viruses were mixed together before addition to the cultures, and the cultures returned to the roller rig 28° C.

After 2 days (48 hrs.) post infection the 5 l of culture was harvested. The total cell count at harvest was 1.58×10E6 cells/ml. (99% viable). The cells were spun out at 2500 rpm, 30 mins., 4° C. in Heraeus Omnifuge 2.0 RS in 250 ml. lots. The supernatant was discarded.

### Partial Co-Purification of CDK2 and Cyclin E

Sf21 cells were resuspended in lysis buffer (50 mM Tris pH 8.2, 10 mM MgCl₂, 1 mM DTT, 10 mM glycerophosphate, 0.1 mM sodium orthovanadate, 0.1 mM NaF, 1 mM PMSF, 1 ug/ml leupeptin and 1 ug/ml aprotinin) and homogenised for 2 mins in a 10 ml Dounce homogeniser. After centrifugation, the supernatant was loaded onto a Poros HQ/M 1.4/100 anion exchange column (PE Biosystems, Hertford, UK). CDK2 and Cyclin E were coeluted at the beginning of a 0-1M NaCl gradient (run in lysis buffer minus protease inhibitors) over 20 column volumes. Co-elution was checked by western blot using both anti-CDK2 and anti-Cyclin E antibodies (Santa Cruz Biotechnology, California, US).

By analogy, assays designed to assess inhibition of CDK1 and CDK4 may be constructed. CDK2 (EMBL Accession No. X62071) may be used together with Cyclin A or Cyclin E (see EMBL Accession No. M73812), and further details for such assays are contained in PCT International Publication No. WO99/21845, the relevant Biochemical & Biological Evaluation sections of which are hereby incorporated by reference.

Although the pharmacological properties of the compounds of the formula (I) vary with structural change, in general activity possessed by compounds of the formula (I) may be demonstrated at IC₅₀concentrations or doses in the range 250 μM to 1 nM.

When tested in the above in-vitro assay the CDK2 inhibitory activity of Example 29was measured as IC₅₀=95 nM.

According to a further aspect of the invention there is provided a pharmaceutical composition which comprises a pyrimidine derivative of the formula (I), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt or in vivo hydrolysable ester thereof, as defined hereinbefore for use in a method of treatment of the human or animal body by therapy.

We have found that the compounds defined in the present invention, or a pharmaceutically acceptable salt or in vivo hydrolysable ester thereof, are effective cell cycle inhibitors (anti-cell proliferation agents), which property is believed to arise from their CDK inhibitory properties. Accordingly the compounds of the present invention are expected to be useful in the treatment of diseases or medical conditions mediated alone or in part by CDK enzymes, i.e. the compounds may be used to produce a CDK inhibitory effect in a warm-blooded animal in need of such treatment. Thus the compounds of the present invention provide a method for treating the proliferation of malignant cells characterised by inhibition of CDK enzymes, i.e. the compounds may be used to produce an anti-proliferative and potentially apoptotic effect mediated alone or in part by the inhibition of CDKs. Particularly, an inhibitory effect is produced by preventing entry into or progression through the S phase by inhibition of CDK2, CDK4 and/or CDK6, especially CDK2 and entry into or progression through M phase by inhibition of CDK1. Apoptotic effects may also be envisaged through down-regulation of RNA polymerase II activity by inhibition of CDK1, CDK7, CDK8 and in particular, CDK9. Such a compound of the invention is expected to possess a wide range of anti-cancer properties as CDKs have been implicated in many common human cancers such as leukaemia and breast, lung, colon, rectal, stomach, prostate, bladder, pancreas and ovarian cancer. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, prostate and pancreas. In particular such compounds of the invention are expected to slow advantageously the growth of primary and recurrent solid tumours of, for example, the colon, breast, prostate, lungs and skin. More particularly such compounds of the invention, or a pharmaceutically acceptable salt or in vivo hydrolysable ester thereof, are expected to inhibit the growth of those primary and recurrent solid tumours which are associated with CDKs, especially those tumours which are significantly dependent on CDKs for their growth and spread, including for example, certain tumours of the colon, breast, prostate, lung, vulva and skin.

It is further expected that a compound of the present invention will possess activity against other cell-proliferation diseases in a wide range of other disease states including leukaemias, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Thus according to this aspect of the invention there is provided a compound of the formula (I), or a pharmaceutically acceptable salt or in vivo hydrolysable ester thereof, as defined hereinbefore for use as a medicament.

In a further aspect of the invention there is provided the use of a compound of the formula (I), or a pharmaceutically acceptable salt or in vivo hydrolysable ester thereof, as defined hereinbefore in the manufacture of a medicament for the production of a cell cycle inhibitory effect.

In one aspect of the invention, where a cell cycle inhibitory effect is referred to this refers to inhibition of CDK1. In a further aspect of the invention, this refers to inhibition of CDK2. In a further aspect of the invention, this refers to inhibition of CDK4. In a further aspect of the invention, this refers to inhibition of CDK5. In a further aspect of the invention, this refers to inhibition of CDK6. In a further aspect of the invention, this refers to inhibition of CDK7. In a further aspect of the invention, this refers to inhibition of CDK8. In a further aspect of the invention, this refers to inhibition of CDK9.

In a further aspect of the invention there is provided the use of a compound of the formula (I), or a pharmaceutically acceptable salt or in vivo hydrolysable ester thereof, as defined hereinbefore in the manufacture of a medicament for the production of an anti-cell-proliferation effect.

In a further aspect of the invention there is provided the use of a compound of the formula (I), or a pharmaceutically acceptable salt or in vivo hydrolysable ester thereof, as defined hereinbefore in the manufacture of a medicament for the production of a CDK2inhibitory effect.

In a further aspect of the invention there is provided the use of a compound of the formula (I), or a pharmaceutically acceptable salt or in vivo hydrolysable ester thereof, as defined hereinbefore in the manufacture of a medicament for the treatment of cancer.

In a further aspect of the invention there is provided the use of a compound of the formula (I), or a pharmaceutically acceptable salt or in vivo hydrolysable ester thereof, as defined hereinbefore in the manufacture of a medicament for the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the manufacture of a medicament for the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided a method of producing a cell cycle inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing a CDK2inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use as a medicament.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a cell cycle inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of an anti-cell-proliferation effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a CDK2 inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of leukaemia or lymphoid malignancies or cancer of the breast, lung, colon, rectum, stomach, liver, kidney, prostate, bladder, pancreas, vulva, skin or ovary.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation,

In a further aspect of the invention there is provided the use of a compound of the formula (I), or a pharmaceutically acceptable salt or in vivo hydrolysable ester thereof, as defined hereinbefore, in the production of a cell cycle inhibitory effect.

In a further aspect of the invention there is provided the use of a compound of the formula (I), or a pharmaceutically acceptable salt or in vivo hydrolysable ester thereof, as defined hereinbefore, in the production of an anti-cell-proliferation effect.

In a further aspect of the invention there is provided the use of a compound of the formula (I), or a pharmaceutically acceptable salt or in vivo hydrolysable ester thereof, as defined hereinbefore, in the production of a CDK2 inhibitory effect.

In a further aspect of the invention there is provided the use of a compound of the formula (I), or a pharmaceutically acceptable salt or in vivo hydrolysable ester thereof, as defined hereinbefore, in the treatment of cancer.

In a further aspect of the invention there is provided the use of a compound of the formula (I), or a pharmaceutically acceptable salt or in vivo hydrolysable ester thereof, as defined hereinbefore in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Preventing cells from entering DNA synthesis by inhibition of essential S-phase initiating activities such as CDK2 initiation may also be useful in protecting normal cells of the body from toxicity of cycle-specific pharmaceutical agents. Inhibition of CDK2 or 4 will prevent progression into the cell cycle in normal cells which could limit the toxicity of cycle-specific pharmaceutical agents which act in S-phase, G2 or mitosis. Such protection may result in the prevention of hair loss normally associated with these agents.

Therefore in a further aspect of the invention there is provided a compound of formula (I) as defined above or a pharmaceutically acceptable salt or in vivo hydrolysable ester thereof for use as a cell protective agent.

Therefore in a further aspect of the invention there is provided a compound of formula (I) as defined above or a pharmaceutically acceptable salt or in vivo hydrolysable ester thereof for use in preventing hair loss arising from the treatment of malignant conditions with pharmaceutical agents.

Examples of pharmaceutical agents for treating malignant conditions that are known to cause hair loss include alkylating agents such as ifosfamide and cyclophosphamide; antimetabolites such as methotrexate, 5-fluorouracil, gemcitabine and cytarabine; vinca alkaloids and analogues such as vincristine, vinbalstine, vindesine, vinorelbine; taxanes such as paclitaxel and docetaxel; topoisomerase I inhibitors such as irinotecan and topotecan; cytotoxic antibiotics such as doxorubicin, daunorubicin, mitoxantrone, actinomycin-D and mitomycin; and others such as etoposide and tretinoin.

In another aspect of the invention, the compound of formula (I), or a pharmaceutically acceptable salt or in vivo hydrolysable ester thereof, may be administered in association with a one or more of the above pharmaceutical agents. In this instance the compound of formula (I) may be administered by systemic or non systemic means. Particularly the compound of formula (I) my may administered by non-systemic means, for example topical administration.

Therefore in an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof.

In an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof in simultaneous, sequential or separate administration with an effective amount of said pharmaceutical agent.

According to a further aspect of the invention there is provided a pharmaceutical composition for use in preventing hair loss arising from the treatment of malignant conditions with pharmaceutical agents which comprises a compound of formula (I), or a pharmaceutically acceptable salt or in vivo hydrolysable ester thereof, and said pharmaceutical agent, in association with a pharmaceutically acceptable diluent or carrier.

According to a further aspect of the present invention there is provided a kit comprising a compound of formula (I), or a pharmaceutically acceptable salt or in vivo hydrolysable ester thereof, and a pharmaceutical agent for treating malignant conditions that is known to cause hair loss.

According to a further aspect of the present invention there is provided a kit comprising:

a) a compound of formula (I), or a pharmaceutically acceptable salt or in vivo hydrolysable ester thereof, in a first unit dosage form;
b) a pharmaceutical agent for treating malignant conditions that is known to cause hair loss; in a second unit dosage form; and
c) container means for containing said first and second dosage forms.

According to another feature of the invention there is provided the use of a compound of the formula (I), or a pharmaceutically acceptable salt or in vivo hydrolysable ester thereof, in the manufacture of a medicament for the prevention of hair loss during treatment of malignant conditions with pharmaceutical agents.

According to a further aspect of the present invention there is provided a combination treatment for the prevention of hair loss comprising the administration of an effective amount of a compound of the formula (I), or a pharmaceutically acceptable salt or in vivo hydrolysable ester thereof, optionally together with a pharmaceutically acceptable diluent or carrier, with the simultaneous, sequential or separate administration of an effective amount of a pharmaceutical agent for treatment of malignant conditions to a warm-blooded animal, such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The CDK inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may cover three main categories of therapeutic agent:

(i) other cell cycle inhibitory agents that work by the same or different mechanisms from those defined hereinbefore;
(ii) cytostatic agents such as antiestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors); and
(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan). According to this aspect of the invention there is provided a pharmaceutical product comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated;
(viii) chemical symbols have their usual meanings; SI units and symbols are used;
(ix) solvent ratios are given in volume:volume (v/v) terms;
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:
BOC tert-butoxy carbonyl;
IPA isopropyl alcohol;
THF tetrahydrofuran;
DIPEA N,N-diisopropylethylamine;
DMAP 4-dimethylaminopyridine;
DMF N,N-dimethylformamide;
DMF-DMA N,N-dimethylformamide dimethylacetal;
DMA dimethylacetamide;
EtOAc ethyl acetate;
MeOH methanol;
ether diethyl ether;
EtOH ethanol;
HATU O-(7-azabenzotriazol-1-yl)-1,1-3,3-tetramethyluronium hexafluorophosphate;
DCM dichloromethane;
TEA triethylamine;
DMSO dimethylsulphoxide;
TFA trifluoroacetic acid; and
RPHPLC reverse phase high performance liquid chromatography;
   (xvii) PTFE filters used for filtration are manufactured by Gelman® and consist of a 0.45 μM membrane filter cup. These are available from Fisher-Scientific UK (Part Code 09730155);
   (xviii) where an SCX-2 or SCX-3 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ; and
   (xix) The nomenclature used for the 3-azabicyclo[3.1.0]hexan-6-amine system is shown below, where (1α, 5α, 6α) refers to the substituents at the 1, 5 and 6 position all being on the same face of the molecule:

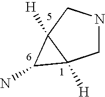

(shown here as all down).

### Example 1

### Benzyl 4-[[4-(2-methyl-3-propan-2-yl-imidazol-4-yl)pyrimidin-2-yl]amino]piperidine-1-carboxylate

(E)-3-Dimethylamino-1-(2-methyl-3-propan-2-yl-imidazol-4-yl)prop-2-en-1-one (Method 24 WO 03/076436, 18.9 g, 85.46 mmol) and benzyl 4-carbamimidamidopiperidine-1-carboxylate (Method 1; 30.7 g, 111 mmol) in 2-methoxyethanol (120 ml) were heated at reflux for 24 hrs. The reaction mixture was allowed to cool to ambient temperature overnight. The resultant precipitate was filtered and washed with a little MeOH, then ether and dried under vacuum to give the required product as a white solid (29.8 g, 80%). NMR (400.132 MHz, CDCl₃) 1.39 (m, 2H), 1.48 (d, 6H), 2.00 (m, 2H), 2.50 (s, 3H), 2.94 (m, 2H), 3.92 (m, 1H), 4.08 (m, 2H), 4.88 (m, 1H), 5.07 (s, 2H), 5.50 (m, 1H), 6.67 (d, 1H), 7.27 (m, 6H), 8.13 (d, 1H); MH+ 435.

### Example 2

### 4-(2-Methyl-3-propan-2-yl-imidazol-4-yl)-N-(4-piperidinyl)pyrimidin-2-amine

Benzyl 4-[[4-(2-methyl-3-propan-2-yl-imidazol-4-yl)pyrimidin-2-yl]amino]piperidine-1-carboxylate (Example 1, 29.8 g, 68.5 mmol) and 10% Pd/C (3 g) in EtOH (500 ml) were stirred at 40° C. under hydrogen at 5 bar pressure for 18 hrs. The catalyst was filtered off and solvent evaporated to a give a clear gum. Trituration with ether gave a white solid which was filtered and dried. (19.2 g, 93%). NMR (400.132 MHz, CDCl₃) 1.34 (m, 2H), 1.44 (s, 1H), 1.49 (d, 6H), 1.99 (m, 2H), 2.50 (s, 3H), 2.65 (m, 2H), 3.06 (m, 2H), 3.84 (m, 1H), 4.91 (m, 1H), 5.57 (m, 1H), 6.65 (d, 1H), 7.24 (s, 1H), 8.12 (d, 1H); MH+ 301.

### Example 3

### N-[1-(3-Chloropropylsulfonyl)-4-piperidinyl]-4-(2-methyl-3-propan-2-yl-imidazol-4-yl)pyrimidin-2-amine

3-Chloropropane sulfonyl chloride (1.5 ml, 12.6 mmol) was slowly added to a suspension of 4-(2-methyl-3-propan-2-yl-imidazol-4-yl)-N-(4-piperidinyl)pyrimidin-2-amine (Example 2; 1.9 g, 6.3 mmol) and TEA (2.6 ml, 12.6 mmol) in DCM (80 ml). The reaction mixture was heated to 40° C. for 90 mins then additional 3-chloropropane sulfonyl chloride (0.75 ml, 6.3 mmol) was added and the reaction stirred for a further 2 hrs. The reaction solution was diluted with DCM (70 ml) and washed with water (150 ml). The aqueous layer was extracted with DCM (4×100 ml). The combined organic extracts were washed with sat. aq NaHCO₃, brine, dried, filtered and evaporated. The resultant material was purified on silica, eluting with 5% MeOH/DCM to give the title compound as a brown solid (770 mg, 28%). MH+ 441.

### Example 4

### N-(1-Benzyl-4-piperidinyl)-4-(2-methyl-3-propan-2-yl-imidazol-4-yl)pyrimidin-2-amine

Benzyl 4-[[4-(2-methyl-3-propan-2-yl-imidazol-4-yl)pyrimidin-2-yl]amino]piperidine-1-carboxylate (Example 1, 7.4 g, 17 mmol) in concentrated hydrochloric acid (50 ml) was heated at 100° C. for 1 hr. The reaction was cooled to ambient temperature and neutralised to pH 11 with 40% aq NaOH. The aqueous solution was extracted with DCM (4×150 ml) and the combined organic extracts washed with brine, dried and evaporated. Purification of the resultant material on silica eluting with 10% MeOH/DCM then 20% 2M ammonia in MeOH/DCM gave two products identified as 4-(2-methyl-3-propan-2-yl-imidazol-4-yl)-N-(4-piperidinyl)pyrimidin-2-amine (Example 2, 2.3 g) and the title compound (2.4 g, 36%). NMR (400.132 MHz) 1.48 (d, 6H), 1.56 (m, 2H), 1.85 (m, 2H), 2.00 (m, 2H), 2.47 (s, 3H), 2.83 (m, 2H), 3.47 (s, 2H), 3.70 (m, 1H), 5.67 (m, 1H), 6.78 (d, 1H), 7.05 (s, 1H), 7.25 (m, 1H), 7.32 (m, 5H), 8.18 (d, 1H); MH+ 391.

### Example 5

### 4-(2-Methyl-3-propan-2-yl-imidazol-4-yl)-N-[1-(3-pyrrolidin-1-ylpropylsulfonyl)-4-piperidinyl]pyrimidin-2-amine

To a solution of N-[1-(3-chloropropylsulfonyl)-4-piperidinyl]-4-(2-methyl-3-propan-2-yl-imidazol-4-yl)pyrimidin-2-amine (Example 3, 70 mg, 0.16 mmol) dissolved in THF (4 ml), was added sodium iodide (5 mg, 0.03 mmol) followed by pyrrolidine (0.05 ml, 0.64 mmol). The reaction was heated by microwave irradiation at 150° C. for 2 hrs. The solvent was evaporated and the resultant material purified by base modified RPHPLC. The resultant material was dissolved in MeOH and added to a SCX-3 column pre-wet with MeOH. The column was flushed with MeOH and the product eluted with 2M ammonia/MeOH. Solvents were evaporated to yield the title compound as a yellow solid (47 mg, 62%). NMR (400.132 MHz, CDCl₃) 1.54 (m, 8H), 1.72 (m, 4H), 1.94 (m, 2H), 2.08 (m, 2H), 2.43 (m, 4H), 2.49 (m, 5H), 2.94 (m, 4H), 3.71 (m, 2H), 3.89 (m, 1H), 4.89 (m, 1H), 5.47 (m, 1H), 6.69 (d, 1H), 7.24 (s, 1H), 8.14 (d, 1H); MH+ 476.

### Examples 6-8

The following compounds were prepared by the procedure of Example 5 and on the same scale, using the appropriate amine starting material.

| Ex | Compound | NMR (400.132 MHz, CDCl₃) | m/z |
|---|---|---|---|
| 6 | N-[1-[3-(4-Methylpiperazin- | 1.49 (d, 6H), 1.57 (m, 2H), 1.91 (m, 2H), | 505 |
| | 1-yl)propylsulfonyl]-4- | 2.08 (m, 2H), 2.22 (s, 3H), 2.38 (m, 10H), | |
| | piperidinyl]-4-(2-methyl-3- | 2.50 (s, 3H), 2.93 (m, 4H), 3.70 (m, 2H), | |
| | propan-2-yl-imidazol-4- | 3.90 (m, 1H), 4.88 (m, 1H), 5.46 (m, 1H), | |
| | yl)pyrimidin-2-amine | 6.69 (d, 1H), 7.24 (s, 1H), 8.14 (d, 1H) | |
| 7 | 4-(2-Methyl-3-propan-2-yl- | 1.49 (d, 6H), 1.57 (m, 2H), 1.92 (m, 2H), | 492 |
| | imidazol-4-yl)-N-[1-(3- | 2.09 (m, 2H), 2.38 (m, 6H), 2.50 (s, 3H), | |
| | morpholin-4-ylpropylsulfonyl)- | 2.93 (m, 4H), 3.63 (m, 4H), 3.70 (m, 2H), | |
| | 4-piperidinyl]pyrimidin- | 3.91 (m, 1H), 4.89 (m, 1H), 5.46 (m, 1H), | |
| | 2-amine | 6.69 (d, 1H), 7.24 (s, 1H), 8.14 (d, 1H) | |
| 8 | 2-[4-[3-[[4-[[4-(2-Methyl-3- | 1.48 (d, 6H), 1.56 (m, 2H), 1.91 (m, 2H), | 535 |
| | propan-2-yl-imidazol-4- | 2.08 (m, 2H), 2.43 (m, 15H), 2.61 (s, 1H), | |
| | yl)pyrimidin-2-yl]amino]-1- | 2.93 (m, 4H), 3.54 (m, 2H), 3.70 (m, 2H), | |
| | piperidinyl]sulfonyl]propyl]piperazin- | 3.90 (m, 1H), 4.89 (m, 1H), 5.46 (m, 1H), | |
| | 1-yl]ethanol | 6.69 (d, 1H), 7.24 (s, 1H), 8.14 (d, 1H) | |

### Example 9

### 2-[3-[[4-[[4-(2-Methyl-3-propan-2-yl-imidazol-4-yl)pyrimidin-2-yl]amino]-1-piperidinyl]sulfonyl]propylamino]butan-1-ol

To a solution of N-[1-(3-chloropropylsulfonyl)-4-piperidinyl]-4-(2-methyl-3-propan-2-yl-imidazol-4-yl)pyrimidin-2-amine (Example 3, 70 mg, 0.16 mmol) dissolved in THF (4 ml), was added sodium iodide (5 mg, 0.03 mmol) followed by 2-amino-1-butanol (0.22 ml, 2.38 mmol). The reaction was heated by microwave irradiation at 150° C. for 2 hrs. The solvent was evaporated and the resultant material purified by base modified RPHPLC. The resultant material was dissolved in MeOH and added to a SCX-3 column pre-wet with MeOH, The column was flushed with MeOH and the product eluted with 2M ammonia/MeOH. Solvents were evaporated to yield the title compound as a white solid (36 mg, 46%). NMR (400.132 MHz, CDCl₃) 0.85 (t, 3H), 1.30-1.49 (m, 8H), 1.57 (m, 2H), 1.91 (m, 2H), 2.08 (m, 2H), 2.47 (m, 4H), 2.63 (m, 1H), 2.80 (m, 1H), 2.95 (m, 4H), 3.24 (m, 1H), 3.56 (m, 1H), 3.70 (m, 2H), 3.91 (m, 1H), 4.92 (m, 1H), 5.46 (m, 1H), 6.69 (d, 1H), 7.24 (s, 1H), 8.14 (d, 1H); MH+ 494.

### Examples 10-14

The following compounds were prepared by the procedure of Example 9 and on the same scale, using the appropriate amine starting material.

| Ex | Compound | NMR (400.132 MHz, CDCl₃) | m/z |
|---|---|---|---|
| 10 | N-[1-[3-(1-Methoxypropan- | 0.94 (d, 3H), 1.49 (d, 6H), 1.56 (m, 2H), | 494 |
| | 2-ylamino)propylsulfonyl]- | 1.90 (m, 2H), 2.08 (m, 2H), 2.50 (s, 3H), 2.68 (m, | |
| | 4- | 2H), 2.80 (m, 1H), 2.97 (m, 4H), 3.14 (m, | |
| | piperidinyl]-4-(2-methyl- | 1H), 3.25 (m, 1H), 3.28 (s, 3H), 3.71 (m, 2H), | |
| | 3-propan-2-yl-imidazol-4- | 3.90 (m, 1H), 4.89 (m, 1H), 5.47 (m, 1H), | |
| | yl)pyrimidin-2-amine | 6.69 (d, 1H), 7.24 (s, 1H), 8.14 (d, 1H) | |
| 11 | 1-[3-[[4-[[4-(2-Methyl-3- | 1.09 (d, 3H), 1.49 (d, 6H), 1.57 (m, 2H), | 480 |
| | propan-2-yl-imidazol-4- | 1.92 (m, 2H), 2.09 (m, 2H), 2.36 (m, 1H), 2.50 (s, | |
| | yl)pyrimidin-2-yl]amino]- | 3H), 2.70 (m, 3H), 2.95 (m, 4H), 3.71 (m, | |
| | 1-piperidinyl]sulfonyl]propylamino]propan- | 3H), 3.91 (m, 1H), 4.92 (m, 1H), 5.46 (m, | |
| | 2-ol | 1H), 6.69 (d, 1H), 7.24 (s, 1H), 8.14 (d, 1H) | |
| 12 | 2-[3-[[4-[[4-(2-Methyl-3- | (399.902 MHz) 0.99 (d, 3H), 1.48 (d, 6H), | 480 |
| | propan-2-yl-imidazol-4- | 1.57 (m, 2H), 1.92 (m, 2H), 2.08 (m, 2H), | |
| | yl)pyrimidin-2-yl]amino]- | 2.50 (s, 3H), 2.61 (m, 1H), 2.71 (m, 1H), | |
| | 1-piperidinyl]sulfonyl]propylamino]propan- | 2.82 (m, 1H), 2.96 (m, 4H), 3.19 (m, 1H), 3.52 (m, | |
| | 1-ol | 1H), 3.70 (m, 2H), 3.91 (m, 1H), 4.93 (m, | |
| | | 1H), 5.46 (m, 1H), 6.69 (d, 1H), 7.24 (s, 1H), | |
| | | 8.14 (d, 1H) | |
| 13 | 3-Methyl-2-[3-[[4-[[4-(2- | 0.83 (d, 3H), 0.90 (d, 3H), 1.49 (d, 6H), | 508 |
| | methyl-3-propan-2-yl- | 1.57 (m, 2H), 1.73 (m, 1H), 1.91 (m, 2H), 2.08 (m, | |
| | imidazol-4-yl)pyrimidin-2- | 2H), 2.31 (m, 1H), 2.50 (s, 3H), 2.63 (m, 1H), | |
| | yl]amino]-1-piperidinyl]sulfonyl]propylamino]butan- | 2.80 (m, 1H), 2.95 (m, 4H), 3.27 (m, 1H), | |
| | 1-ol | 3.55 (m, 1H), 3.71 (m, 2H), 3.91 (m, 1H), | |
| | | 4.91 (m, 1H), 5.46 (m, 1H), 6.69 (d, 1H), | |
| | | 7.24 (s, 1H), 8.14 (d, 1H) | |
| 14 | 2-Methyl-2-[3-[[4-[[4-(2- | 1.00 (s, 6H), 1.49 (d, 6H), 1.57 (m, 2H), | 494 |
| | methyl-3-propan-2-yl- | 1.88 (m, 2H), 2.08 (m, 2H), 2.50 (s, 3H), 2.60 (m, | |
| | imidazol-4-yl)pyrimidin-2- | 2H), 2.96 (m, 4H), 3.23 (s, 2H), 3.70 (m, 2H), | |
| | yl]amino]-1-piperidinyl]sulfonyl]propylamino]propan- | 3.91 (m, 1H), 4.92 (m, 1H), 5.46 (m, 1H), | |
| | 1-ol | 6.69 (d, 1H), 7.24 (s, 1H), 8.14 (d, 1H) | |

### Example 15

### N-[1-[3-(2-Methoxy ethylamino)propylsulfonyl]-4-piperidinyl]-4-(2-methyl-3-propan-2-yl-imidazol-4-yl)pyrimidin-2-amine

To a solution of N-[1-(3-chloropropylsulfonyl)-4-piperidinyl]-4-(2-methyl-3-propan-2-yl-imidazol-4-yl)pyrimidin-2-amine (Example 3, 42 mg, 0.09 mmol) dissolved in THF (3 ml), was added sodium iodide (3 mg, 0.02 mmol) followed by 2-methoxymethanamine (0.14 ml, 1.4 mmol). The reaction was heated by microwave irradiation at 150° C. for 2 hrs. Reaction mixture was filtered and the filtrate evaporated. The resultant material was dissolved in DCM and purified on silica eluting with 5% 2M ammonia in MeOH/DCM. Fractions containing product were combined and evaporated to give a gum, which was triturated with ether to give the title compound as a yellow solid (36 mg, 79%). NMR (400.132 MHz, CDCl₃) 1.49 (d, 6H), 1.60 (m, 2H), 2.11 (m, 4H), 2.51 (s, 3H), 2.94 (m, 6H), 3.09 (m, 2H), 3.32 (s, 3H), 3.57 (m, 2H), 3.72 (m, 2H), 3.91 (m, 1H), 4.99 (m, 1H), 5.47 (m, 1H), 6.69 (d, 1H), 7.26 (s, 1H), 8.14 (d, 1H); MH+ 480.

### Example 16

### 3-[[4-[[4-(2-Methyl-3-propan-2-yl-imidazol-4-yl)pyrimidin-2-yl]amino]-1-piperidinyl]sulfonyl]propan-1-ol

N-[1-(3-Chloropropylsulfonyl)-4-piperidinyl]-4-(2-methyl-3-propan-2-yl-imidazol-4-yl)pyrimidin-2-amine (Example 3, 50 mg, 0.11 mmol) was dissolved in EtOH (4 ml), sodium acetate (47 mg, 0.57 mmol) and sodium iodide (5 mg, 0.03 mmol) were added and the reaction was heated by microwave irradiation at 140° C. for 1 hr. The reaction mixture was filtered and washed with EtOH. 2M NaOH (3 ml) was added to the filtrate and the solution was stirred at ambient temperature for 1 hr. The reaction solution was neutralised to pH 7with 2M aq HCL and solvents evaporated. The resultant material was partitioned between DCM and water. The contents of the organic phase were purified on silica, eluting on a gradient of 5-10% MeOH/DCM. Fractions containing the product were combined and evaporated to a gum, which was triturated with ether to give the title compound as a yellow solid (17 mg, 36%). NMR (399.902 MHz, CDCl₃) 1.55 (m, 8H), 1.70 (m, 1H), 2.05 (m, 4H), 2.50 (s, 3H), 2.93 (m, 2H), 3.02 (m, 2H), 3.73 (m, 4H), 3.90 (m, 1H), 4.91 (m, 1H), 5.46 (m, 1H), 6.69 (d, 1H), 7.24 (s, 1H), 8.14 (d, 1H), MH+ 423.

### Example 17

### 4-[[4-(2-Methyl-3-propan-2-yl-imidazol-4-yl)pyrimidin-2-yl]amino]piperidine-1-sulfonamide

4-(2-Methyl-3-propan-2-yl-imidazol-4-yl)-N-(4-piperidinyl)pyrimidin-2-amine (Example 2, 60 mg, 0.2 mmol) and sulfamide (192 mg, 2 mmol) in dioxan were heated at reflux for 16 hrs. The reaction was cooled to ambient temperature and diluted with water (50 ml) and sat. aq. NaHCO₃ (50 ml). The aqueous solution was extracted with DCM (5×50 ml). Combined organics were evaporated and the resultant material dissolved in DCM with a little MeOH and purified on silica, eluting with a gradient of 0-10% MeOH/DCM. Fractions containing product were combined and evaporated to give the title compound as a white solid (40 mg, 53%). NMR (400.132 MHz, CDCl₃) 1.49 (d, 6H), 1.61 (m, 2H), 2.10 (m, 2H), 2.50 (s, 3H), 2.84 (m, 2H), 3.64 (m, 2H), 3.88 (m, 1H), 4.39 (s, 2H), 4.96 (m, 1H), 5.48 (m, 1H), 6.69 (d, 1H), 7.25 (s, 1H), 8.14 (d, 1H); MH+ 380.

### Example 18

### N,N-Dimethyl-4-[[4-(2-methyl-3-propan-2-yl-imidazol-4-yl)pyrimidin-2-yl]amino]piperidine-1-sulphonamide

Dimethylsulfamoyl chloride (0.026 ml, 0.24 mmol) in DCM (1 ml) was added dropwise to a stirred solution of 4-(2-methyl-3-propan-2-yl-imidazol-4-yl)-N-(4-piperidinyl)pyrimidin-2-amine (Example 2; 60 mg, 0.2 mmol) and TEA (0.084 ml, 0.6 mmol) in DCM (1 ml). The solution was stirred at ambient temperature for 16 hrs. Water (2 ml) was added then the mixture filtered through a PTFE cup and purified on silica, eluting with a gradient of 0-5% MeOH/DCM to give a solid. Trituration with ether then re-evaporation gave the title compound as a colourless solid (62 mg, 76%). NMR (400.132 MHz, CDCl₃) 1.49 (d, 6H), 1.55 (m, 2H), 2.06 (m, 2H), 2.50 (s, 3H), 2.76 (s, 6H), 2.92 (m, 2H), 3.62 (m, 2H), 3.88 (m, 1H), 4.88 (m, 1H), 5.49 (m, 1H), 6.69 (d, 1H), 7.25 (s, 1H), 8.14 (d, 1H); MH+ 408.

### Example 19

### 4-[[4-(2-Methyl-3-propan-2-yl-imidazol-4-yl)pyrimidin-2-yl]amino]piperidine-1-carboxamide

4-(2-Methyl-3-propan-2-yl-imidazol-4-yl)-N-(4-piperidinyl)pyrimidin-2-amine (Example 2; 150 mg, 0.5 mmol) was dissolved in THF (5 ml) and a solution of potassium cyanate (163 mg, 2.0 mmol) in water (1 ml) was added. The resulting solution was cooled to 0° C. and 1M HCl (2 ml) was added. The reaction mixture was stirred at 0° C. for 2 hrs, then warmed to ambient temperature overnight. The reaction mixture was applied to a 5 g SCX-3 column, washing with water (2×10 ml), MeOH (2×10 ml) then eluting with 3.5N NH₃/MeOH (2×10 ml). The solvent was removed in vacuo to give a colourless gum which was triturated with ether, filtered and dried to give the title compound as a colourless solid (142 mg, 83%). NMR (400 MHz, CDCl₃) 1.44-1.59 (m, 8H), 2.10 (d, 2H), 2.57 (s, 3H), 3.02 (t, 2H), 3.88-4.07 (m, 3H), 4.42 (brs, 2H), 4.90-5.03 (m, 1H), 5.49-5.64 (m, 1H), 6.75 (d, 1H), 7.31 (s, 1H), 8.21 (d, 1H); MH+ 344.

### Example 20

### N,N-Dimethyl-4-[[4-(2-methyl-3-propan-2-yl-imidazol-4-yl)pyrimidin-2-yl]amino]piperidine-1-carboxamide

4-(2-Methyl-3-propan-2-yl-imidazol-4-yl)-N-(4-piperidinyl)pyrimidin-2-amine (Example 2; 150 mg, 0.50 mmol) and TEA (0.1 ml, 0.75 mmol) were dissolved in DCM (5 ml). Dimethylcarbamyl chloride (0.06 ml) was added and the reaction stirred under an inert atmosphere for 16 hrs. Trisamine resin (150 mg) was added, the reaction mixture was agitated for 1 hr then filtered through a plug of diatomaceous earth and evaporated in vacuo. Trituration with ether gave the title compound as a colourless solid (81 mg, 44%). NMR (400 MHz) 1.39-1.57 (m, 8H), 1.87 (d, 2H), 2.48 (s, 3H), 2.69-2.84 (m, 8H), 3.57 (d, 2H), 3.79-3.93 (brs, 1H), 5.74-6.54 (brs, 1H), 6.80 (d, 1H), 6.97-7.23 (brs, 1H), 7.34 (s, 1H), 8.12 (d, 1H); MH+ 372.

### Example 21

### N-Methyl-4-[[4-(2-methyl-3-propan-2-yl-imidazol-4-yl)pyrimidin-2-yl]amino]piperidine-1-carboxamide

The title compound was prepared by the procedure of Example 109 and on the same scale, using 4-(2-methyl-3-propan-2-yl-imidazol-4-yl)-N-(4-piperidinyl)pyrimidin-2-amine (Example 2) as the starting material. NMR (CDCl₃) 2.19 (m, 7H), 2.27 (q, 1H), 2.49 (t, 1H), 2.79 (s, 3H), 2.82 (d, 3H), 2.97 (dd, 2H), 3.86-4.05 (m, 3H), 4.46 (d, 1H), 5.04-5.24 (brs, 1H), 5.48-5.68 (m, 1H), 6.74 (d, 1H), 7.32 (s, 2H), 8.19 (d, 1H).

### Example 22

### (4-Methyl-1,4-diazepan-1-yl)-[4-[[4-(2-methyl-3-propan-2-yl-imidazol-4-yl)pyrimidin-2-yl]amino]-1-piperidinyl]methanone

4-Nitrophenylchloroformate (111 mg, 0.55 mmol) was added to a stirred solution of 4-(2-methyl-3-propan-2-yl-imidazol-4-yl)-N-(4-piperidinyl)pyrimidin-2-amine (Example 2; 151 mg, 0.5 mmol) and TEA (0.15 ml, 1.10 mmol) in dioxane (5 ml) under an inert atmosphere. After 2 hrs N-methylhomopiperazine (0.069 g, 0.6 mmol) was added and the reaction heated at 80° C. for 4 hrs. After which the mixture was evaporated in vacuo and the residue dissolved in EtOAc (10 ml) and washed with 1N NaOH (5×10 ml), then brine. The organic layer was dried, filtered and evaporated to a give a solid. The residue was loaded onto a SCX-2 column, washed with water, MeOH and then 3.5N NH₃/MeOH to give the title compound as a yellow solid (78 mg, 35%). NMR (CDCl₃, 400 MHz) 1.44-1.58 (m, 8H), 1.87-1.96 (m, 2H), 2.08 (d, 2H), 2.36 (s, 3H), 2.52-2.60 (m, 5H), 2.62-2.70 (m, 2H), 2.88 (t, 2H), 3.42-3.52 (m, 4H), 3.60 (d, 2H), 3.89-4.02 (m, 1H), 4.98 (brd, 1H), 5.51-5.67 (appbrs, 1H), 6.74 (d, 1H), 7.31 (s, 1H), 8.20 (d, 1H); MH+ 441.

### Examples 23-28

The following compounds were prepared by the procedure of Example 22 and on the same scale, by using the appropriate amine and with additional purification by RPHPLC.

| Ex | Compound | NMR (400.132 MHz, CDCl₃) | m/z |
|---|---|---|---|
| 23 | N-(1-Methyl-4- | 1.41-1.58 (m, 8H), 1.91-2.19 (m, 6H), 2.29 (s, | 441.6 |
| | piperidinyl)-4-[[4-(2- | 3H), 2.57 (s, 3H), 2.81 (d, 2H), 2.96 (t, 2H), | |
| | methyl-3-propan-2-yl- | 3.61-3.74 (m, 1H), 3.86-4.05 (m, 3H), 4.29 (d, | |
| | imidazol-4-yl)pyrimidin-2- | 1H), 4.96 (d, 1H), 5.49-5.67 (s, 1H), 6.75 (d, | |
| | yl]amino]piperidine-1- | 1H), 7.31 (s, 1H), 8.20 (d, 1H) | |
| | carboxamide | | |
| 24 | N-[[(2S)-1- | 1.09 (t, 4H), 1.40-1.78 (m, 12H), | 455.6 |
| | Ethylpyrrolidin-2- | 1.79-1.93 (m, 1H), 2.07 (d, 2H), 2.12-2.30 (m, 2H), | |
| | yl]methyl]-4-[[4-(2- | 2.57 (s, 3H), 2.58-2.68 (m, 1H), 2.72-2.86 (m, 1H), | |
| | methyl-3-propan-2-yl- | 2.97 (t, 2H), 3.17 (brd, 2H), 3.34-3.46 (m, | |
| | imidazol-4-yl)pyrimidin-2- | 1H), 3.79-4.08 (m, 3H), 4.97 (d, 1H), | |
| | yl]amino]piperidine-1- | 5.18-5.43 (brs, 1H), 5.50-5.65 (brs, 1H), 6.74 (d, | |
| | carboxamide | 1H), 7.31 (s, 1H), 8.20 (d, 1H) | |
| 25 | N-[[(2R)-1- | 1.09 (t, 4H), 1.40-1.78 (m, 10H), | 455.6 |
| | Ethylpyrrolidin-2- | 1.79-1.93 (m, 1H), 2.07 (d, 2H), 2.12-2.30 (m, 2H), | |
| | yl]methyl]-4-[[4-(2- | 2.57 (s, 3H), 2.58-2.68 (m, 1H), 2.72-2.86 (m, 1H), | |
| | methyl-3-propan-2-yl- | 2.97 (t, 2H), 3.17 (brd, 2H), 3.34-3.46 (m, | |
| | imidazol-4-yl)pyrimidin-2- | 1H), 3.79-4.08 (m, 3H), 4.97 (appd, 1H), | |
| | yl]amino]piperidine-1- | 5.18-5.43 (brs, 1H), 5.50-5.65 (brs, 1H), | |
| | carboxamide | 6.74 (d, 1H), 7.31 (s, 1H), 8.20 (d, 1H) | |
| 26 | N-(2-Dimethylamino- | | 415.6 |
| | ethyl)-4-[[4-(2-methyl-3- | | |
| | propan-2-yl-imidazol-4- | | |
| | yl)pyrimidin-2- | | |
| | yl]amino]piperidine-1- | | |
| | carboxamide | | |
| 27 | [(3S)-3-Dimethylamino- | 1.39-1.59 (m, 8H), 1.69-1.81 (m, 1H), | 441.6 |
| | pyrrolidin-1-yl]-[4-[[4-(2- | 2.00-2.13 (m, 3H), 2.27 (s, 6H), 2.57 (s, 3H), | |
| | methyl-3-propan-2-yl- | 2.59-2.69 (m, 1H), 2.81-3.01 (m, 2H), 3.23 (t, 1H), | |
| | imidazol-4-yl)pyrimidin-2- | 3.40-3.58 (m, 3H), 3.73 (t, 2H), 3.93-4.05 (m, | |
| | yl]amino]-1- | 1H), 4.99 (brd, 1H), 5.51-5.67 (brs, 1H), | |
| | piperidinyl]methanone | 6.74 (d, 1H), 7.31 (s, 1H), 8.20 (d, 1H) | |
| 28 | 4-[[4-(2-Methyl-3-propan- | 1.41-1.58 (m, 9H), 1.74-1.81 (m, 3H), | 441.6 |
| | 2-yl-imidazol-4- | 2.03-2.11 (m, 2H), 2.49-2.55 (m, 4H), 2.57 (s, 3H), | |
| | yl)pyrimidin-2-yl]amino]- | 2.62 (t, 2H), 2.96 (t, 2H), 3.34 (q, 2H), | |
| | N-(2-pyrrolidin-1- | 3.87-4.05 (m, 3H), 4.97 (d, 1H), 5.17-5.26 (brs, | |
| | ylethyl)piperidine-1- | 1H), 5.49-5.64 (m, 1H), 6.74 (d, 1H), 7.31 (s, | |
| | carboxamide | 1H), 8.21 (d, 1H) | |

### Example 29

### 1-[4-[[4-(2-Methyl-3-propan-2-yl-imidazol-4-yl)pyrimidin-2-yl]amino]-1-piperidinyl]ethanone

4-(2-Methyl-3-propan-2-yl-imidazol-4-yl)-N-(4-piperidinyl)pyrimidin-2-amine (Example 2, 83 mg, 0.28 mmol) was suspended in DCM (2 ml). TEA (0.077 ml, 0.55 ml) was added, followed by acetic anhydride (0.03 ml, 0.30 mmol) in DCM (1 ml) over a period of one minute. The reaction was stirred at ambient temperature for 3 days. The reaction solution was washed with sat. aq. NaHCO₃, filtered through a PTFE cup and solvents evaporated to give the title compound as a white foam (72 mg, 77%). NMR (400.132 MHz, CDCl₃) 1.39 (m, 2H), 1.49 (d, 6H), 2.05 (m, 5H), 2.50 (s, 3H), 2.78 (m, 1H), 3.14 (m, 1H), 3.76 (m, 1H), 3.98 (m, 1H), 4.45 (m, 1H), 4.90 (m, 1H), 5.49 (m, 1H), 6.69 (d, 1H), 7.25 (s, 1H), 8.14 (d, 1H); MH+ 343.

### Example 30

### 1-[4-[[4-(2-Methyl-3-propan-2-yl-imidazol-4-yl)pyrimidin-2-yl]amino]1-piperidinyl]-4-morpholin-4-yl-butan-1-one

4-(2-Methyl-3-propan-2-yl-imidazol-4-yl)-N-(4-piperidinyl)pyrimidin-2-amine (Example 2, 60 mg, 0.2 mmol), 4-morpholin-4-yl butanoic acid hydrochloride (Method 3; 50 mg, 0.24 mmol), HATU (91 mg, 0.24 mmol) and DIPEA (0.14 ml, 0.8 mmol) in DMF (2 ml) were stirred at ambient temperature overnight. Solvents were evaporated and the resultant material was partitioned between DCM (2 ml) and sat. aq. NaHCO₃ (2 ml), gravity filtered through a PTFE cup and evaporated. The resultant material was dissolved in DCM and purified on silica eluting with a shallow gradient of 0-10% MeOH/DCM. Fractions containing product were combined and evaporated to give the title compound as a white glassy solid (64 mg, 70%). NMR (400.132 MHz, CDCl₃) 1.39 (m, 2H), 1.49 (d, 6H), 1.78 (m, 2H), 2.05 (m, 2H), 2.39 (m, 8H), 2.50 (s, 3H), 2.78 (m, 1H), 3.12 (m, 1H), 3.65 (m, 4H), 3.82 (m, 1H), 3.98 (m, 1H), 4.45 (m, 1H), 4.89 (m, 1H), 5.49 (m, 1H), 6.69 (d, 1H), 7.25 (s, 1H), 8.14 (d, 1H); MH+ 456.

### Example 31

### N-(1-Methyl-4-piperidinyl)-4-(2-methyl-3-propan-2-yl-imidazol-4-yl)pyrimidin-2-amine

4-(2-Methyl-3-propan-2-yl-imidazol-4-yl)-N-(4-piperidinyl)pyrimidin-2-amine (Example 2, 50 mg, 0.17 mmol) was dissolved in THF (2 ml). Acetic acid (0.01 ml, 0.17 mmol) was added which resulted in precipitation of material. Aqueous formaldehyde (37%>, 1 ml) was added causing dissolution of the precipitate. The reaction was stirred at ambient temperature for 30 mins. Sodium triacetoxyborohydride (100 mg) was added and the reaction stirred for a further 2 hrs. Solvents were evaporated and the resultant material neutralised with sat. aq. NaHCO₃ and extracted with DCM (15 ml), filtered through a PTFE cup and added to a silica column. The column was eluted with a shallow gradient of 0-20% 2M ammonia in MeOH/DCM. Fractions containing product were combined and evaporated to give the title compound as a white solid. (33 mg, 63%). NMR (400.132 MHz, CDCl₃) 1.52 (m, 8H), 2.01 (m, 4H), 2.23 (s, 3H), 2.50 (s, 3H), 2.76 (m, 2H), 3.75 (m, 1H), 4.90 (m, 1H), 5.57 (m, 1H), 6.65 (d, 1H), 7.24 (s, 1H), 8.12 (d, 1H); MH+ 315.

### Example 32

### 4-(2-Methyl-3-propan-2-yl-imidazol-4-yl)-N-(1-propan-2-yl-4-piperidinyl)pyrimidin-2-amine

4-(2-Methyl-3-propan-2-yl-imidazol-4-yl)-N-(4-piperidinyl)pyrimidin-2-amine (Example 2, 70 mg, 0.23 mmol) was suspended in acetone (3 ml). Acetic acid (0.013 ml, 0.23 mmol) was added followed by DCM (1 ml) to aid solubility. The reaction was stirred at ambient temperature for 30 mins. Sodium triacetoxyborohydride (99 mg, 0.47 mmol) was added and the reaction was left to stir for 16 hrs. A further addition of sodium triacetoxyborohydride (200 mg, 0.94 mmol) was made and the reaction heated at 40° C. overnight. Solvents were evaporated and the residues neutralised with sat. aq. NaHCO₃, shaken with DCM and filtered through a PTFE cup. The DCM solvent was evaporated to a give clear oil. Ether was added and the solution re-evaporated. The resultant oil placed under high vacuum to give the title compound as a white solid (45 mg, 56%). NMR (400.132 MHz, CDCl₃) 0.98 (d, 6H), 1.49 (m, 8H), 1.99 (m, 2H), 2.21 (m, 2H), 2.50 (s, 3H), 2.67 (m, 1H), 2.80 (m, 2H), 3.75 (m, 1H), 4.91 (m, 1H), 5.57 (m, 1H), 6.64 (d, 1H), 7.24 (s, 1H), 8.12 (d, 1H); MH+ 343.

### Example 33

### 4-(2-Methyl-3-propan-2-yl-imidazol-4-yl)-N-(1-phenethyl-4-piperidinyl)pyrimidin-2-amine

4-(2-Methyl-3-propan-2-yl-imidazol-4-yl)-N-(4-piperidinyl)pyrimidin-2-amine (Example 2, 70 mg, 0.23 mmol), 2-bromoethylbenzene (65 mg, 0.35 mmol) and TEA (0.097 ml, 0.7 mmol) in DMF (2 ml) were heated at 50° C. for 65 hrs. The temperature was increased to 90° C. for 2 hrs then the solvents were evaporated. The resultant material was dissolved in DCM and chromatographed on silica eluting with a shallow gradient of 0-10% MeOH/DCM. Fractions containing product were combined and evaporated to a gum, ether was added, re-evaporated and dried under vacuum, to give the title compound as a white solid (33 mg, 35%). NMR (400.132 MHz, CDCl₃) 1.59 (m, 8H), 2.08 (m, 2H), 2.20 (m, 2H), 2.57 (s, 3H), 2.62 (m, 2H), 2.82 (m, 2H), 2.97 (m, 2H), 3.86 (m, 1H), 4.99 (m, 1H), 5.63 (m, 1H), 6.72 (d, 1H), 7.21 (m, 3H), 7.29 (m, 3H), 8.20 (d, 1H); MH+ 405.

### Example 34

### tert-Butyl 4-[[4-(2-methyl-3-propan-2-yl-imidazol-4-yl)pyrimidin-2-yl]amino]piperidine-1-carboxylate

(2-Methylpropan-2-yl)oxycarbonyl tert-butyl carbonate (44 mg, 0.2 mmol) dissolved in THF (1 ml) was added to a solution of 4-(2-methyl-3-propan-2-yl-imidazol-4-yl)-N-(4-piperidinyl)pyrimidin-2-amine (Example 2, 60 mg, 0.2 mmol) dissolved in THF (3 ml). The reaction mixture was stirred at ambient temperature for 90 mins. Solvents were evaporated and the residue dissolved in DCM and passed through a silica column, eluting with a gradient of 0-10% MeOH/DCM. Fractions containing product were combined and evaporated to a give clear gum. Ether was added to the gum and re-evaporated to give the title compound as a white foam (47 mg, 59%). NMR (400.132 MHz, CDCl₃) 1.36 (m, 11H), 1.48 (d, 6H), 1.97 (m, 2H), 2.50 (s, 3H), 2.85 (m, 2H), 3.93 (m, 3H), 4.88 (m, 1H), 5.52 (m, 1H), 6.67 (d, 1H), 7.25 (s, 1H), 8.13 (d, 1H).

### Example 35

### N-(1-Ethenylsulfonyl-4-piperidinyl)-4-(2-methyl-3-propan-2-yl-imidazol-4-yl)pyrimidin-2-amine

TEA (0.7 ml, 5 mmol) was added to a solution of 4-(2-methyl-3-propan-2-yl-imidazol-4-yl)-N-(4-piperidinyl)pyrimidin-2-amine (Example 2; 500 mg, 1.7 mmol) in DCM (25 ml). 2-Chloroethane sulfonyl chloride (0.26 ml, 2.5 mmol), in a small volume of DCM, was added dropwise to the solution giving a colour change from colourless to yellow. Upon complete addition a solid had formed. After 30 mins the solid material was filtered off. The filtrate was diluted with DCM and washed with water. The aqueous phase was extracted with DCM and the combined organics washed with brine, dried and evaporated. The resultant material was dissolved in DCM and chromatographed on silica eluting with a gradient of 0-10% MeOH/DCM. Fractions containing product were combined and evaporated to give the title compound as an off-white solid (218 mg, 34%). MH+ 391.

### Example 36

### N-[1-[2-(4-Methylpiperazin-1-yl)ethylsulfonyl]-4-piperidinyl]-4-(2-methyl-3-propan-2-yl-imidazol-4-yl)pyrimidin-2-amine

1-Methyl piperazine (0.06 ml, 0.54 mmol) was added to a solution of N-(1-ethenylsulfonyl-4-piperidinyl)-4-(2-methyl-3-propan-2-yl-imidazol-4-yl)pyrimidin-2-amine (Example 35, 72 mg, 0.18 mmol) in 1:1 THF/DCM (3 ml). The reaction mixture was stirred at ambient temperature for 72 hrs. Solvents were evaporated and the resultant material purified by base modified RPHPLC to yield the title compound as a gum (60 mg, 68%). NMR (400.132 MHz, CDCl₃) 1.49 (d, 6H), 1.56 (m, 2H), 2.08 (m, 2H), 2.22 (s, 3H), 2.39-2.46 (m, 8H), 2.50 (s, 3H), 2.77 (m, 2H), 2.93 (m, 2H), 3.06 (m, 2H), 3.71 (m, 2H), 3.90 (m, 1H), 4.88 (m, 1H), 5.46 (m, 1H), 6.69 (d, 1H), 7.24 (s, 1H), 8.14 (d, 1H); MH+ 491.

### Examples 37-62

The following compounds were prepared by the procedure of Example 36 and on the same scale, using the appropriate amine starting material.

| Ex | Compound | NMR (400.132 MHz, CDCl₃) | m/z |
|---|---|---|---|
| 37 | 4-(2-Methyl-3-propan-2-yl- | 1.48 (d, 6H), 1.56 (m, 2H), 1.74 (m, 4H), | 462 |
| | imidazol-4-yl)-N-[1-(2- | 2.08 (m, 2H), 2.48 (m, 7H), 2.85 (m, 2H), | |
| | pyrrolidin-1-ylethylsulfonyl)- | 2.93 (m, 2H), 3.09 (m, 2H), 3.71 (m, 2H), | |
| | 4-piperidinyl]pyrimidin-2- | 3.89 (m, 1H), 4.88 (m, 1H), 5.46 (m, 1H), | |
| | amine | 6.69 (d, 1H), 7.24 (s, 1H), 8.14 (d, 1H) | |
| 38 | N-[1-[2-(2-Methoxyethylamino)ethylsulfonyl]- | 1.48 (d, 6H), 1.57 (m, 2H), 2.08 (m, 2H), | 466 |
| | 4- | 2.50 (s, 3H), 2.74 (m, 2H), 2.92 (m, 2H), | |
| | piperidinyl]-4-(2-methyl-3- | 3.05 (m, 4H), 3.29 (s, 3H), 3.42 (m, 2H), | |
| | propan-2-yl-imidazol-4- | 3.71 (m, 2H), 3.90 (m, 1H), 4.89 (m, 1H), | |
| | yl)pyrimidin-2-amine | 5.46 (m, 1H), 6.69 (d, 1H), 7.24 (s, 1H), | |
| | | 8.14 (d, 1H) | |
| 39 | 4-(2-Methyl-3-propan-2-yl- | 1.49 (d, 6H), 1.57 (m, 2H), 2.09 (m, 2H), | 494 |
| | imidazol-4-yl)-N-[1-(2- | 2.52 (s, 3H), 2.61 (m, 4H), 2.70 (m, 4H), | |
| | thiomorpholin-4-ylethylsulphonyl)- | 2.81 (m, 2H), 2.93 (m, 2H), 3.04 (m, 2H), | |
| | 4-piperidinyl]pyrimidin- | 3.70 (m, 2H), 3.91 (m, 1H), 5.00 (m, 1H), | |
| | 2-amine | 5.45 (m, 1H), 6.69 (d, 1H), 7.26 (s, 1H), | |
| | | 8.15 (d, 1H) | |
| 40 | 4-(2-Methyl-3-propan-2-yl- | | 476 |
| | imidazol-4-yl)-N-[1-[2-(1- | | |
| | piperidinyl)ethylsulphonyl]- | | |
| | 4-piperidinyl]pyrimidin-2- | | |
| | amine | | |
| 41 | N-[1-[2-(Methyl-propan-2-yl- | 0.97 (d, 6H), 1.53 (m, 8H), 2.09 (m, 2H), | 464 |
| | amino)ethylsulphonyl]-4- | 2.17 (s, 3H), 2.50 (s, 3H), 2.79 (m, 3H), | |
| | piperidinyl]-4-(2-methyl-3- | 2.91 (m, 2H), 3.02 (m, 2H), 3.72 (m, 2H), | |
| | propan-2-yl-imidazol-4- | 3.89 (m, 1H), 4.89 (m, 1H), 5.47 (m, 1H), | |
| | yl)pyrimidin-2-amine | 6.69 (d, 1H), 7.24 (s, 1H), 8.14 (d, 1H) | |
| 42 | N-[1-[2-(Azetidin-1- | | 448 |
| | yl)ethylsulphonyl]-4- | | |
| | piperidinyl]-4-(2-methyl-3- | | |
| | propan-2-yl-imidazol-4- | | |
| | yl)pyrimidin-2-amine | | |
| 43 | 4-(2-Methyl-3-propan-2-yl- | | 478 |
| | imidazol-4-yl)-N-[1-(2- | | |
| | morpholin-4-ylethylsulphonyl)- | | |
| | 4-piperidinyl]pyrimidin- | | |
| | 2-amine | | |
| 44 | N-[1-[2-(4-Methyl-1- | 0.87 (d, 3H), 1.18 (m, 2H), 1.31 (m, 1H), | 490 |
| | piperidinyl)ethylsulphonyl]- | 1.48 (d, 6H), 1.56 (m, 4H), 1.99 (m, 2H), | |
| | 4-piperidinyl]-4-(2-methyl-3- | 2.08 (m, 2H), 2.50 (s, 3H), 2.77 (m, 4H), | |
| | propan-2-yl-imidazol-4- | 2.93 (m, 2H), 3.08 (m, 2H), 3.71 (m, 2H), | |
| | yl)pyrimidin-2-amine | 3.90 (m, 1H), 4.90 (m, 1H), 5.46 (m, 1H), | |
| | | 6.69 (d, 1H), 7.24 (s, 1H), 8.14 (d, 1H) | |
| 45 | N-[1-[2-(Azepan-1- | | 490 |
| | yl)ethylsulphonyl]-4- | | |
| | piperidinyl]-4-(2-methyl-3- | | |
| | propan-2-yl-imidazol-4- | | |
| | yl)pyrimidin-2-amine | | |
| 46 | N-[1-(2-Diethylamino- | | 464 |
| | ethylsulphonyl)-4- | | |
| | piperidinyl]-4-(2-methyl-3- | | |
| | propan-2-yl-imidazol-4- | | |
| | yl)pyrimidin-2-amine | | |
| 47 | 4-[2-[[4-[[4-(2-Methyl-3- | 1.49 (d, 6H), 1.58 (m, 2H), 2.09 (m, 2H), | 491 |
| | propan-2-yl-imidazol-4- | 2.50 (s, 3H), 2.67 (m, 2H), 2.87 (m, 2H), | |
| | yl)pyrimidin-2-yl]amino]-1- | 2.95 (m, 2H), 3.06 (m, 2H), 3.12 (s, 2H), | |
| | piperidinyl]sulphonyl]ethyl]piperazin- | 3.32 (m, 2H), 3.70 (m, 2H), 3.92 (m, 1H), | |
| | 2-one | 5.04 (m, 1H), 5.45 (m,1H), 5.93 (s, 1H), | |
| | | 6.69 (d, 1H), 7.25 (s, 1H), 8.14 (d, 1H) | |
| 48 | 4-(2-Methyl-3-propan-2-yl- | | 492 |
| | imidazol-4-yl)-N-[1-[2-(1,4- | | |
| | oxazepan-4-yl)ethylsulphonyl]- | | |
| | 4-piperidinyl]pyrimidin- | | |
| | 2-amine | | |
| 49 | N-[1-[2-[(3R)-3- | | 480 |
| | Fluoropyrrolidin-1- | | |
| | yl]ethylsulfonyl]-4- | | |
| | piperidinyl]-4-(2-methyl-3- | | |
| | propan-2-yl-imidazol-4- | | |
| | yl)pyrimidin-2-amine | | |
| 50 | N-[1-[2-(4-Fluoro-1- | | 494 |
| | piperidinyl)ethylsulphonyl]- | | |
| | 4-piperidinyl]-4-(2-methyl-3- | | |
| | propan-2-yl-imidazol-4- | | |
| | yl)pyrimidin-2-amine | | |
| 51 | N-[1-[2-(7- | | 488 |
| | Azabicyclo[2.2.1]hept-7- | | |
| | yl)ethylsulphonyl]-4- | | |
| | piperidinyl]-4-(2-methyl-3- | | |
| | propan-2-yl-imidazol-4- | | |
| | yl)pyrimidin-2-amine | | |
| 52 | N-[1-[2-(Cyclopropyl- | | 462 |
| | methyl-amino)ethylsulphonyl]- | | |
| | 4-piperidinyl]-4- | | |
| | (2-methyl-3-propan-2-yl- | | |
| | imidazol-4-yl)pyrimidin-2- | | |
| | amine | | |
| 53 | N-[1-[2-(Cyclopropylmethyl- | | 476 |
| | methyl-amino)ethylsulphonyl]- | | |
| | 4-piperidinyl]-4- | | |
| | (2-methyl-3-propan-2-yl- | | |
| | imidazol-4-yl)pyrimidin-2- | | |
| | amine | | |
| 54 | 1-[2-[[4-[[4-(2-Methyl-3- | 1.49 (d, 6H), 1.59 (m, 2H), 1.70 (m, 2H), | 519 |
| | propan-2-yl-imidazol-4- | 1.84 (m, 2H), 2.07 (m, 5H), 2.50 (s, 3H), | |
| | yl)pyrimidin-2-yl]amino]-1- | 2.75 (m, 2H), 2.87 (m, 2H), 2.96 (m, 2H), | |
| | piperidinyl]sulphonyl]ethyl]piperidine- | 3.06 (m, 2H), 3.68 (m, 2H), 3.92 (m, 1H), | |
| | 4-carboxamide | 4.93 (m, 1H), 5.21 (m, 1H), 5.46 (m, 2H), | |
| | | 6.69 (d, 1H), 7.24 (s, 1H), 8.13 (d, 1H) | |
| 55 | 1-[4-[2-[[4-[[4-(2-Methyl-3- | | 519 |
| | propan-2-yl-imidazol-4- | | |
| | yl)pyrimidin-2-yl]amino]-1- | | |
| | piperidinyl]sulphonyl]ethyl]piperazin- | | |
| | 1-yl]ethanone | | |
| 56 | 1-[2-[[4-[[4-(2-Methyl-3- | 1.48 (d, 6H), 1.58 (m, 2H), 2.09 (m, 2H), | 505 |
| | propan-2-yl-imidazol-4- | 2.50 (s, 3H), 2.60 (m, 6H), 2.92 (m, 4H), | |
| | yl)pyrimidin-2-yl]amino]-1- | 3.03 (m, 2H), 3.24 (m, 2H), 3.69 (m, 2H), | |
| | piperidinyl]sulphonyl]ethyl]- | 3.92 (m, 1H), 5.01 (m, 1H), 5.45 (m, 1H), | |
| | 1,4-diazepan-5-one | 5.86 (m, 1H), 6.69 (d, 1H), 7.25 (s, 1H), | |
| | | 8.14 (d, 1H) | |
| 57 | 1-[4-[2-[[4-[[4-(2-Methyl-3- | | 533 |
| | propan-2-yl-imidazol-4- | | |
| | yl)pyrimidin-2-yl]amino]-1- | | |
| | piperidinyl]sulphonyl]ethyl]- | | |
| | 1,4-diazepan-1-yl]ethanone | | |
| 58 | 4-(2-Methyl-3-propan-2-yl- | | 518 |
| | imidazol-4-yl)-N-[1-[2-(4- | | |
| | propyl-1-piperidinyl)ethylsulphonyl]- | | |
| | 4-piperidinyl]pyrimidin- | | |
| | 2-amine | | |
| 59 | 4-(2-Methyl-3-propan-2-yl- | | 508 |
| | imidazol-4-yl)-N-[1-[2-(1,4- | | |
| | thiazepan-4-yl)ethylsulphonyl]- | | |
| | 4-piperidinyl]pyrimidin- | | |
| | 2-amine | | |
| 60 | N-[1-[2-(2- | | 502 |
| | Azabicyclo[2.2.2]oct-2- | | |
| | yl)ethylsulfonyl]-4- | | |
| | piperidinyl]-4-(2-methyl-3- | | |
| | propan-2-yl-imidazol-4- | | |
| | yl)pyrimidin-2-amine | | |
| 61 | 1-[2-[[4-[[4-(2-Methyl-3- | | 501 |
| | propan-2-yl-imidazol-4- | | |
| | yl)pyrimidin-2-yl]amino]-1- | | |
| | piperidinyl]sulphonyl]ethyl]piperidine- | | |
| | 4-carbonitrile | | |
| 62 | N-[1-[2-[(3S)-3- | | 480 |
| | Fluoropyrrolidin-1- | | |
| | yl]ethylsulfonyl]-4- | | |
| | piperidinyl]-4-(2-methyl-3- | | |
| | propan-2-yl-imidazol-4- | | |
| | yl)pyrimidin-2-amine | | |

### Example 63

### 4-(2-Methyl-3-propan-2-yl-imidazol-4-yl)-N-(1-methylsulfonyl-4-piperidinyl)pyrimidin-2-amine

2-Chloro-4-(2-methyl-3-propan-2-yl-imidazol-4-yl)pyrimidine (Method 5; 105 mg, 0.44 mmol), 1-methylsulfonylpiperidin-4-amine TFA salt (Example 162, WO 04/069139, 156 mg, 0.53 mmol), DIPEA (0.23 ml, 1.33 mmol) and IPA (3 ml) were combined and heated by microwave irradiation at 140° C. for 30 mins. The reaction mixture was passed through a polymer supported bicarbonate cartridge and then re-heated at 140° C. for 30 mins. A further portion of 1-methylsulfonylpiperidin-4-amine TFA salt (Example 162, WO 04/069139, 220 mg, 0.76 mmol) was dissolved in MeOH and added to an SCX-2 column pre-wet with MeOH. The column was flushed with MeOH and the free base eluted with 2M ammonia in MeOH. The eluent was evaporated and the resultant material added to the reaction as a solution in DIPEA (0.2 ml). The reaction was heated at 150° C. for 5 hrs. A resultant precipitate was collected by filtration, dissolved in DCM and purified on silica, eluting with 10% MeOH/DCM. The filtrate from the reaction was evaporated, dissolved in DCM and purified on silica eluting on a shallow gradient of 0-5% MeOH/DCM then 5% MeOH/DCM. Fractions containing the required product from both columns were combined and evaporated to give the title compound as a white solid. (70 mg, 42%). NMR (400.132 MHz) 1.55 (d, 6H), 1.64 (m, 2H), 2.03 (m, 2H), 2.53 (s, 3H), 2.88 (m, 2H), 2.94 (s, 3H), 3.64 (m, 2H), 3.89 (m, 1H), 5.67 (m, 1H), 6.87 (d, 1H), 7.20 (s, 1H), 7.40 (s, 1H), 8.27 (d, 1H); MH+ 379.

### Example 64

### Ethyl 4-[[4-(2-methyl-3-propan-2-yl-imidazol-4-yl)pyrimidin-2-yl]amino]piperidine-1-carboxylate

2-Chloro-4-(2-methyl-3-propan-2-yl-imidazol-4-yl)pyrimidine (Method 5; 70 mg, 0.3 mmol), ethyl-4-amino-1-piperidinecarboxylate (103 mg, 0.6 mmol), TEA (0.084 ml, 0.6 mmol) and IPA (3 ml) were combined and heated by microwave irradiation at 160° C. for 5 hrs. Solvents were evaporated; the residue was dissolved in DCM and washed with water, filtered through a PTFE cup and solvents evaporated. The resultant material was purified by base modified RPHPLC to give the title compound as a white solid (52 mg, 46%). NMR (400.132 MHz, CDCl₃) 1.20 (t, 3H), 1.38 (m, 2H), 1.49 (d, 6H), 1.99 (m, 2H), 2.50 (s, 3H), 2.91 (m, 2H), 3.91 (m, 1H), 4.07 (m, 4H), 4.89 (m, 1H), 5.51 (m, 1H), 6.68 (d, 1H), 7.25 (s, 1H), 8.14 (d, 1H); MH+ 373.

### Examples 65

### Endo-8-methyl-N-[4-(2-methyl-3-propan-2-yl-imidazol-4-yl)pyrimidin-2-yl]-8-azabicyclo[3.2.1]octan-3-amine

2-Chloro-4-(2-methyl-3-propan-2-yl-imidazol-4-yl)pyrimidine (Method 5; 70 mg, 0.3 mmol), endo-8-methyl-8-azabicyclo[3.2.1]octan-3-amine dihydrochloride (128 mg, 0.6 mmol), TEA (0.25 ml, 1.8 mmol) and IPA (3 ml) were combined and heated by microwave irradiation at 160° C. for 8 hrs. Solvents were evaporated; the residue was dissolved in DCM and extracted with water. The aqueous phase was added to a SCX-3 column, which had been pre-wet with MeOH. The column was flushed with MeOH and the product eluted with 2M ammonia in MeOH and the eluent evaporated. The resultant material was purified by base modified RPHPLC to give the title compound as a white solid (10 mg, 10%). NMR (400.132 MHz, CDCl₃) 1.46 (d, 6H), 1.75 (m, 2H), 1.87 (m, 2H), 2.08 (m, 2H), 2.23 (m, 5H), 2.49 (s, 3H), 3.13 (m, 2H), 4.05 (m, 1H), 5.33 (m, 1H), 5.64 (m, 1H), 6.67 (d, 1H), 7.26 (s, 1H), 8.11 (d, 1H); MH+ 341.

### Example 66

### N-[1-(2-Methoxyethyl)-4-piperidinyl]-4-(2-methyl-3-propan-2-yl-imidazol-4-amine

2-Chloro-4-(2-methyl-3-propan-2-yl-imidazol-4-yl)pyrimidine (Method 5; 70 mg, 0.3 mmol), 1-(2-methoxyethyl)piperidin-4-amine (95 mg, 0.6 mmol), TEA (0.084 ml, 0.6 mmol) and IPA (3 ml) were combined and heated by microwave irradiation at 160° C. for 5 hrs. Solvents were evaporated; the residue was dissolved in DCM and washed with water, filtered through a PTFE cup and solvents evaporated. The resultant material was purified by base modified RPHPLC to give the title compound as a gum (34 mg, 32%). NMR (400.132 MHz, CDCl₃) 1.49 (d, 6H), 1.56 (m, 2H), 1.98 (m, 2H), 2.12 (m, 2H), 2.52 (m, 5H), 2.87 (m, 2H), 3.29 (s, 3H), 3.45 (m, 2H), 3.77 (m, 1H), 4.90 (m, 1H), 5.56 (m, 1H), 6.65 (d, 1H), 7.24 (s, 1H), 8.12 (d, 1H); MH+ 359.

### Example 67

### 4-(2-Methyl-3-propan-2-yl-imidazol-4-yl)-N-(1-propyl-4-piperidinyl)pyrimidin-2-amine

2-Chloro-4-(2-methyl-3-propan-2-yl-imidazol-4-yl)pyrimidine (Method 5; 70 mg, 0.3 mmol), 1-propylpiperidin-4-amine (85 mg, 0.6 mmol), TEA (0.084 ml, 0.6 mmol) and IPA (3 ml) were combined and heated by microwave irradiation at 160° C. for 5 hrs. Solvents were evaporated and the residue dissolved in DCM (10 ml) and washed with water (10 ml), then filtered through a PTFE cup and the DCM layer evaporated. The resultant material was dissolved in DCM and purified on silica eluting with a gradient of 0-5% 2M ammonia in MeOH/DCM. Fractions containing pure product were combined and evaporated to give the title compound as a white solid (45 mg, 44%). NMR (400.132 MHz, CDCl₃) 0.84 (t, 3H), 1.47 (m, 10H), 2.01 (m, 4H), 2.23 (m, 2H), 2.50 (s, 3H), 2.82 (m, 2H), 3.76 (m, 1H), 4.90 (m, 1H), 5.58 (m, 1H), 6.65 (d, 1H), 7.24 (s, 1H), 8.12 (d, 1H); MH+ 343.

### Example 68

### tert-Butyl 4-[2-[4-[[4-(2-methyl-3-propan-2-yl-imidazol-4-yl)pyrimidin-2-yl]-amino]-1-piperidinyl]-2-oxo-ethyl]piperidine-1-carboxylate

4-(2-Methyl-3-propan-2-yl-imidazol-4-yl)-N-(4-piperidinyl)pyrimidin-2-amine (Example 2, 100 mg, 0.33 mmol), 1-((1,1-dimethylethoxy)carbonyl)-4-piperidineacetic acid (97 mg, 0.4 mmol), HATU (152 mg, 0.4 mmol), DIPEA (0.23 ml, 1.33 mmol) and DMF (4 ml) were combined and stirred at ambient temperature overnight. Solvents were evaporated and the resultant material partitioned between DCM (2 ml) and sat. aq. NaHCO₃ (2 ml), gravity filtered through a PTFE cup and evaporated. The resultant material was dissolved in DCM and purified on silica eluting with a shallow gradient of 0-5% MeOH/DCM. Fractions containing pure material were combined and evaporated to give the title compound (23 mg, 13%). NMR (400.132 MHz, CDCl₃) 1.07 (m, 2H), 1.37 (m, 11H), 1.49 (d, 6H), 1.68 (m, 2H), 1.94 (m, 1H), 2.05 (m, 2H), 2.20 (m, 2H), 2.51 (s, 3H), 2.67 (m, 2H), 2.78 (m, 1H), 3.11 (m, 1H), 3.80 (m, 1H), 4.00 (m, 3H), 4.47 (m, 1H), 4.88 (m, 1H), 5.48 (m, 1H), 6.69 (d, 1H), 7.25 (s, 1H), 8.15 (d, 1H); MH+ 526.

### Examples 69-79

The following compounds were prepared by the procedure of Example 68 and on the same scale, using the appropriate acid starting material.

| Ex | Compound | NMR (400.132 MHz, CDCl₃) | m/z |
|---|---|---|---|
| 69 | tert-Butyl 4-[3-[4-[[4-(2- | 1.37 (m, 11H), 1.49 (d, 6H), 2.05 (m, 2H), | 541 |
| | methyl-3-propan-2-yl- | 2.37 (m, 4H), 2.48 (m, 5H), 2.67 (m, 2H), | |
| | imidazol-4-yl)pyrimidin-2- | 2.78 (m, 1H), 3.12 (m, 1H), 3.36 (m, 4H), | |
| | yl]amino]-1-piperidinyl]-3- | 3.80 (m, 1H), 3.98 (m, 1H), 4.44 (m, 1H), | |
| | oxo-propyl]piperazine-1- | 4.87 (m, 1H), 5.48 (m, 1H), 6.69 (d, 1H), | |
| | carboxylate | 7.25 (s, 1H), 8.14 (d, 1H) | |
| 70 | tert-Butyl 4-[3-[4-[[4-(2- | 1.05 (m, 2H), 1.38 (m, 12H), 1.52 (m, 8H), | 540 |
| | methyl-3-propan-2-yl- | 1.61 (m, 2H), 2.05 (m, 2H), 2.30 (m, 2H), | |
| | imidazol-4-yl)pyrimidin-2- | 2.51 (s, 3H), 2.61 (m, 2H), 2.78 (m, 1H), | |
| | yl]amino]-1-piperidinyl]-3- | 3.11 (m, 1H), 3.78 (m, 1H), 4.00 (m, 3H), | |
| | oxo-propyl]piperidine-1- | 4.45 (m, 1H), 4.98 (m, 1H), 5.49 (m, 1H), | |
| | carboxylate | 6.69 (d, 1H), 7.26 (s, 1H), 8.15 (d, 1H) | |
| 71 | tert-Butyl 4-methyl-4-[4-[[4- | 1.23 (s, 3H), 1.40 (m, 13H), 1.50 (d, 6H), | 526 |
| | (2-methyl-3-propan-2-yl- | 2.06 (m, 4H), 2.51 (s, 3H), 2.96 (m, 2H), | |
| | imidazol-4-yl)pyrimidin-2- | 3.17 (m, 2H), 3.58 (m, 2H), 4.00 (m, 1H), | |
| | yl]amino]piperidine-1- | 4.26 (m, 2H), 4.90 (m, 1H), 5.49 (m, 1H), | |
| | carbonyl]piperidine-1- | 6.69 (d, 1H), 7.26 (s, 1H), 8.15 (d, 1H) | |
| | carboxylate | | |
| 72 | tert-Butyl (3S)-3-[2-[4-[[4-(2- | 1.16 (m, 1H), 1.38 (m, 12H), 1.49 (d, 6H), | 526 |
| | methyl-3-propan-2-yl- | 1.56 (m, 1H), 1.82 (m, 1H), 2.06 (m, 4H), | |
| | imidazol-4-yl)pyrimidin-2- | 2.25 (m, 1H), 2.51 (s, 3H), 2.63 (m, 1H), | |
| | yl]amino]-1-piperidinyl]-2- | 2.80 (m, 2H), 3.11 (m, 1H), 3.76 (m, 3H), | |
| | oxo-ethyl]piperidine-1- | 3.98 (m, 1H), 4.47 (m, 1H), 4.90 (m, 1H), | |
| | carboxylate | 5.49 (m, 1H), 6.69 (d, 1H), 7.25 (s, 1H), | |
| | | 8.15 (d, 1H) | |
| 73 | tert-Butyl (3R)-3-[2-[4-[[4- | 1.16 (m, 1H), 1.38 (m, 12H), 1.49 (d, 6H), | 526 |
| | (2-methyl-3-propan-2-yl- | 1.57 (m, 1H), 1.82 (m, 1H), 2.05 (m, 4H), | |
| | imidazol-4-yl)pyrimidin-2- | 2.25 (m, 1H), 2.51 (s, 3H), 2.62 (m, 1H), | |
| | yl]amino]-1-piperidinyl]-2- | 2.79 (m, 2H), 3.11 (m, 1H), 3.76 (m, 3H), | |
| | oxo-ethyl]piperidine-1- | 3.98 (m, 1H), 4.47 (m, 1H), 4.89 (m, 1H), | |
| | carboxylate | 5.49 (m, 1H), 6.69 (d, 1H), 7.25 (s, 1H), | |
| | | 8.15 (d, 1H) | |
| 74 | 2-Dimethylamino-1-[4-[[4- | (DMSO) 1.37 (m, 2H), 1.50 (d, 6H), | 386 |
| | (2-methyl-3-propan-2-yl- | 1.91 (m, 2H), 2.20 (s, 6H), 2.70 (m, 1H), 3.09 (m, | |
| | imidazol-4-yl)pyrimidin-2- | 3H), 3.93 (m, 1H), 4.07 (m, 1H), 4.31 (m, | |
| | yl]amino]-1- | 1H), 5.13 (m, 1H), 6.80 (d, 1H), 7.12 (br.s, | |
| | piperidinyl]ethanone | 1H), 7.35 (s, 1H), 8.20 (d, 1H) | |
| 75 | 3-Dimethylamino-1-[4-[[4- | (DMSO) 1.49 (m, 2H), 1.50 (d, 6H), | 400 |
| | (2-methyl-3-propan-2-yl- | 1.81 (m, 2H), 2.44 (s, 3H), 2.63 (m, 1H), 2.72 (m, | |
| | imidazol-4-yl)pyrimidin-2- | 1H), 2.83 (m, 1H), 3.11 (m, 1H), 3.92 (m, | |
| | yl]amino]-1- | 2H), 4.33 (m, 1H), 5.62 (m, 1H), 6.81 (d, | |
| | piperidinyl]propan-1-one | 1H), 7.16 (br.s, 1H), 7.37 (s, 1H), 8.21 (d, | |
| | | 1H) | |
| 76 | 4-Dimethylamino-1-[4-[[4- | (DMSO) 1.39 (m, 2H), 1.51 (d, 6H), | 414 |
| | (2-methyl-3-propan-2-yl- | 1.84 (m, 2H), 1.93 (m, 2H), 2.42 (m, 2H), 2.54 (s, | |
| | imidazol-4-yl)pyrimidin-2- | 3H), 2.79 (s, 6H), 3.08 (m, 2H), 3.12 (m, | |
| | yl]amino]-1- | 1H), 3.86 (m, 1H), 3.99 (m, 1H), 4.32 (m, | |
| | piperidinyl]butan-1-one | 1H), 5.62 (m, 1H), 6.83 (d, 1H), 7.23 (br.s, | |
| | | 1H), 7.51 (s, 1H), 8.27 (d, 1H) | |
| 77 | (1-Methyl-3-piperidinyl)-[4- | 1.44 (m, 3H), 1.57 (s, 6H), 1.78 (m, 2H), | 426 |
| | [[4-(2-methyl-3-propan-2-yl- | 2.13 (m, 4H), 2.36 (s, 3H), 2.58 (s, 3H), | |
| | imidazol-4-yl)pyrimidin-2- | 2.87 (m, 4H), 3.20 (m, 1H), 3.95 (m, 1H), | |
| | yl]amino]-1- | 4.07 (m, 1H), 4.52 (m, 1H), 4.96 (m, 1H), | |
| | piperidinyl]methanone | 5.57 (m, 1H), 6.77 (d, 1H), 7.31 (s, 1H), 8.21 (s, | |
| | | 1H) | |
| 78 | [4-[[4-(2-Methyl-3-propan-2- | 1.43 (m, 2H), 1.55 (d, 6H), 1.62 (m, 2H), | 412 |
| | yl-imidazol-4-yl)pyrimidin-2- | 1.86 (m, 4H), 2.10 (m, 2H), 2.22 (m, 1H), | |
| | yl]amino]-1-piperidinyl]-(1- | 2.38 (s, 3H), 2.56 (s, 3H), 2.86 (m, 1H), | |
| | methylpyrrolidin-2- | 3.08 (m, 1H), 3.15 (m, 2H), 4.11 (m, 2H), | |
| | yl)methanone | 4.54 (m, 1H), 4.97 (m, 1H), 5.57 (m, 1H), | |
| | | 6.77 (d, 1H), 7.32 (s, 1H), 8.21 (d, 1H) | |
| 79 | tert-Butyl 2-[4-[[4-(2-methyl- | 1.45 (s, 9H) overlapping 1.45 (m, 2H), | 514 |
| | 3-propan-2-yl-imidazol-4- | 1.54 (d, 6H), 2.12 (m, 2H), 2.57 (s, 3H), 3.00 (m, | |
| | yl)pyrimidin-2- | 1H), 3.18 (m, 2H), 3.53 (m, 2H), 3.90 (m, | |
| | yl]amino]piperidine-1- | 2H), 4.07 (m, 2H), 4.48 (m, 1H), 4.96 (m, | |
| | carbonyl]morpholine-4- | 1H), 5.55 (m, 1H), 6.76 (d, 1H), 7.31 (s, | |
| | carboxylate | 1H), 8.20 (d, 1H) | |

### Example 80

### 1-[4-[[4-(2-Methyl-3-propan-2-yl-imidazol-4-yl)pyrimidin-2-yl]amino]-1-piperidinyl]-2-(4-piperidinyl)ethanone

tert-Butyl 4-[2-[4-[[4-(2-methyl-3-propan-2-yl-imidazol-4-yl)pyrimidin-2-yl]amino]-1-piperidinyl]-2-oxo-ethyl]piperidine-1-carboxylate (Example 68, 170 mg, 0.33 mmol) was dissolved in DCM (3 ml) and an equal volume of TFA added. The reaction was stirred at ambient temperature for 3 hrs then added to a 5 g SCX-3 column pre-wet with MeOH (2 column volumes). The column was eluted with MeOH (2 column volumes) and product eluted with 2M ammonia in MeOH. The basic eluent was evaporated to give the title compound as a glass (46 mg, 27%). NMR (500.133 MHz) 1.10 (m, 2H), 1.44 (m, 2H), 1.51 (d, 6H), 1.62 (m, 2H), 1.81 (m, 1H), 1.93 (m, 2H), 2.22 (m, 2H), 2.47 (s, 3H), 3.99 (m, 1H), 4.10 (m, 1H, 5.58 (m, 1H), 6.62 (d, 1H), 6.76 (d, 1H), 7.28 (s, 1H), 8.19 (d, 1H); MH+ 426.

### Examples 81-85

The following compounds were prepared by the procedure of Example 80 and on the same scale.

| Ex | Compound | NMR (500.133 MHz) | m/z | SM |
|---|---|---|---|---|
| 81 | 1-[4-[[4-(2-Methyl-3- | 1.46 (m, 2H), 1.51 (d, 6H), 1.93 (m, | 441 | Example |
| | propan-2-yl-imidazol-4- | 2H), 2.33 (m, 4H), 2.47 (m, 5H), | | 69 |
| | yl)pyrimidin-2- | 2.55 (m, 2H), 2.70 (m, 4H), 3.98 (m, 1H), | | |
| | yl]amino]-1-piperidinyl]- | 4.09 (m, 1H, 5.58 (m, 1H), 6.62 (d, | | |
| | 3-piperazin-1-yl-propan- | 1H), 6.76 (d, 1H), 7.28 (s, 1H), | | |
| | 1-one | 8.19 (d, 1H) | | |
| 82 | 1-[4-[[4-(2-Methyl-3- | 1.03 (m, 2H), 1.35 (m, 1H), 1.47 (m, | 440 | Example |
| | propan-2-yl-imidazol-4- | 4H), 1.51 (d, 6H), 1.61 (m, 2H), | | 70 |
| | yl)pyrimidin-2- | 1.93 (m, 2H), 2.31 (m, 2H), 2.47 (s, 3H), | | |
| | yl]amino]-1-piperidinyl]- | 3.98 (m, 1H), 4.09 (m, 1H), 5.58 (m, | | |
| | 3-(4-piperidinyl)propan- | 1H), 6.62 (d, 1H), 6.76 (d, 1H), | | |
| | 1-one | 7.28 (s, 1H), 8.19 (d, 1H) | | |
| 83 | (4-Methyl-4-piperidinyl)- | 1.29 (s, 3H), 1.47 (m, 2H), 1.50 (d, | 426 | Example |
| | [4-[[4-(2-methyl-3- | 6H), 1.65 (m, 2H), 1.96 (m, 2H), | | 71 |
| | propan-2-yl-imidazol-4- | 2.21 (m, 2H), 2.47 (s, 3H), 3.00 (m, 4H), | | |
| | yl)pyrimidin-2- | 3.15 (m, 2H), 4.02 (m, 1H), 4.19 (m, | | |
| | yl]amino]-1- | 2H), 5.54 (m, 1H), 6.76 (d, 1H), | | |
| | piperidinyl]methanone | 7.28 (s, 1H), 8.19 (d, 1H) | | |
| 84 | 1-[4-[[4-(2-Methyl-3- | 1.25 (m, 1H), 1.46 (m, 2H), 1.50 (d, | 426 | Example |
| | propan-2-yl-imidazol-4- | 6H), 1.66 (m, 1H), 1.80 (m, 2H), | | 72 |
| | yl)pyrimidin-2- | 1.94 (m, 2H), 2.19 (m, 1H), 2.33 (m, 2H), | | |
| | yl]amino]-1-piperidinyl]- | 2.47 (s, 3H), 2.61 (m, 1H), 2.76 (m, | | |
| | 2-[(3S)-3- | 1H), 3.19 (m, 1H), 3.27 (m, 1H), | | |
| | piperidinyl]ethanone | 3.99 (m, 1H), 4.09 (m, 1H), 5.55 (m, 1H), | | |
| | | 6.77 (d, 1H), 7.28 (s, 1H), 8.19 (d, 1H) | | |
| 85 | 1-[4-[[4-(2-Methyl-3- | 1.25 (m, 1H), 1.46 (m, 2H), 1.50 (d, | 426 | Example |
| | propan-2-yl-imidazol-4- | 6H), 1.66 (m, 1H), 1.80 (m, 2H), | | 73 |
| | yl)pyrimidin-2- | 1.94 (m, 2H), 2.19 (m, 1H), 2.33 (m, 2H), | | |
| | yl]amino]-1-piperidinyl]- | 2.47 (s, 3H), 2.62 (m, 1H), 2.77 (m, | | |
| | 2-[(3R)-3- | 1H), 3.19 (m, 1H), 3.28 (m, 1H), | | |
| | piperidinyl]ethanone | 3.99 (m, 1H), 4.09 (m, 1H), 5.55 (m, 1H), | | |
| | | 6.77 (d, 1H), 7.28 (s, 1H), 8.19 (d, 1H) | | |

### Example 86

### [4-[[4-(2-Methyl-3-propan-2-yl-imidazol-4-yl)pyrimidin-2-yl]amino]-1-piperidinyl]-morpholin-2-yl-methanone

4M HCl in dioxane (4 ml) was added to a solution of tert-butyl 2-[4-[[4-(2-methyl-3-propan-2-yl-imidazol-4-yl)pyrimidin-2-yl]amino]piperidine-1-carbonyl]morpholine-4-carboxylate (Example 79; 0.3 g, 0.58 mmol) in DCM (4 ml). After stirring for 16 hrs 2M NaOH was added to adjust to pH 12 and the aqueous layer was extracted with DCM (3×15 ml). The combined organics were dried, filtered and concentrated in vacuo to give a colourless foam. Purification by flash chromatography on silica, eluting with 0 to 10% MeOH in DCM gave the title compound as a colourless foam (0.19 g, 79%). NMR (CDCl₃, 400.132 MHz) 1.46 (m, 2H), 1.56 (d, 6H), 2.12 (m, 2H), 2.57 (s, 3H), 2.89 (m, 3H), 3.20 (m, 2H), 3.67 (m, 1H), 3.84 (m, 1H), 4.04 (m, 2H), 4.21 (m, 1H), 4.47 (m, 1H), 4.98 (m, 1H), 5.53 (m, 1H), 6.74 (d, 1H), 7.31 (s, 1H), 8.21 (d, 1H); MH+ 414.

### Example 87

### tert-Butyl 3-[[4-(2-methyl-3-propan-2-yl-imidazol-4-yl)pyrimidin-2-yl]amino]pyrrolidine-1-carboxylate

(E)-3-Dimethylamino-1-(2-methyl-3-propan-2-yl-imidazol-4-yl)prop-2-en-1-one (Method 24 WO 03/076436, 1.85 g, 8.36 mmol), tert-butyl 3-carbamimidamidopyrrolidine-1-carboxylate (Method 2; 2.1 g, 9.1 mmol) and 2-methoxyethanol (20 ml) were combined and heated at reflux for 24 hrs. The reaction mixture was cooled to ambient temperature and evaporated to yield a yellow viscous gum. Ether (˜100 ml) was added and mixture shaken & sonicated. A solid precipitate was collected by suction filtration and dried under vacuum to give the title compound as a beige solid (863 mg, 27%) The filtrate was evaporated and the resultant gum treated with ether (˜25 ml) A solid precipitate was collected by suction filtration and dried under vacuum to give additional title compound as a white solid which was purified by flash chromatography on silica (12 g cartridge) eluting with 0-10% MeOH in DCM. The fractions containing product were combined and evaporated to give additional title compound as a white solid (122 mg, 3.5%). NMR (400.132 MHz) 1.40 (m, 9H), 1.49 (d, 6H), 1.91 (m, 1H), 2.11 (m, 1H), 2.48 (s, 3H), 3.19 (m, 1H), 3.45 (m, 1H), 3.55 (m, 1H), 4.32 (m, 1H), 5.69 (m, 1H), 6.86 (d, 1H), 7.35-7.43 (m, 2H), 8.23 (d, 1H); MH+ 387.3.

### Example 88

### 4-(2-Methyl-3-propan-2-yl-imidazol-4-yl)-N-pyrrolidin-3-yl-pyrimidin-2-amine

tert-Butyl 3-[[4-(2-methyl-3-propan-2-yl-imidazol-4-yl)pyrimidin-2-yl]amino]pyrrolidine-1-carboxylate (Example 87; 855 mg, 2.21 mmol) was dissolved in DCM (10 ml) and TFA (2.5 ml) added. The reaction mixture was stirred at ambient temperature for 3 hrs and then poured directly onto an SCX-2 cartridge (20 g) and washed with DCM (100 ml) and MeOH (100 ml). Products were eluted from the cartridge with 2M ammonia in MeOH (100 ml). Evaporation of the basic fraction gave an amber gum (˜500 mg), which was purified by flash chromatography on silica, eluting with 0-15% gradient of 2M ammonia/MeOH in DCM. Product containing fractions were evaporated to yield a yellow gum, which was triturated/sonicated with ether to afford an off white solid which was collected by vacuum filtration and dried to give the title compound as an off white solid (230 mg, 36%). NMR (400.132 MHz) 1.49 (d, 6H), 1.66 (m, 1H), 2.02 (s, 1H), 2.48 (s, 3H), 2.74 (m, 2H), 2.94 (m, 2H), 4.25 (m, 1H), 5.68 (m, 1H), 6.80 (d, 1H), 7.11 (br s, 1H), 7.35 (s, 1H), 8.20 (d, 1H); MH+ 287.4.

### Example 89

### 4-(2-Methyl-3-propan-2-yl-imidazol-4-yl)-N-(1-methylsulphonylpyrrolidin-3-yl)pyrimidin-2-amine

Catalytic DMAP (5 mg) and DIPEA (0.07 ml, 0.42 mmol) were added to a stirred solution of 4-(2-methyl-3-propan-2-yl-imidazol-4-yl)-N-pyrrolidin-3-yl-pyrimidin-2-amine (Example 88; 62 mg, 0.22 mmol) in DCM (2 ml). Methane sulfonyl chloride (0.025 ml, 0.32mmol) was then added and reaction mixture stirred at ambient temperature for 16 hrs. After which additional DCM (20 ml) and water (15 ml) were added and the mixture shaken before pouring through a phase separating cartridge. The organic eluent was evaporated to dryness to give a pale yellow gum, which was combined with the aqueous phase and concentrated in vacuo. The yellow gum obtained was dissolved in DCM (15 ml), treated with magnesium sulphate, filtered and the filtrate was purified by flash chromatography on silica, eluting with a gradient of 0-10% MeOH in DCM. The residue obtained was triturated with ether to give a solid which was filtered and dried in vacuo to give the title compound as a pale yellow solid (23 mg, 29%). NMR (400.132 MHz) 1.55 (d, 6H), 2.04 (m, 1H), 2.26 (m, 1H), 2.54 (s, 3H), 2.95 (s, 3H), 3.25 (m, 1H), 3.49-3.62 (m, 3H), 4.46 (m, 1H), 5.69 (m, 1H), 6.93 (d, 1H), 7.41-7.49 (m, 2H), 8.31 (d, 1H); MH+ 365.3.

### Example 90

### 3-[[4-(2-Methyl-3-propan-2-yl-imidazol-4-yl)pyrimidin-2-yl]amino]pyrrolidine-1-sulphonamide

A solution of 4-(2-methyl-3-propan-2-yl-imidazol-4-yl)-N-pyrrolidin-3-yl-pyrimidin-2-amine (Example 88; 62 mg, 0.22 mmol) and sulfamide (102 mg, 1.06 mmol) in 1,4-dioxane (2 ml) was heated by microwave irradiation at 130° C. for 30 mins. The reaction mixture was evaporated and sat. aq. NaHCO₃ (10 ml) added to the residue. The aqueous layer was extracted with DCM (2×10 ml) then the combined organics were washed with brine, dried over magnesium sulphate, filtered and evaporated. Purification by flash chromatography on silica, eluting with a gradient of 0-10% MeOH in DCM gave the title compound as a colourless solid (51 mg, 63%). NMR (400.132 MHz) 1.50 (d, 6H), 1.94 (m, 1H), 2.18 (m, 1H), 2.48 (s, 3H), 3.04 (m, 1H), 3.19 (m, 1H), 3.33 (m, 1H), 3.46 (m, 1H), 4.39 (m, 1H), 5.65 (m, 1H), 6.75 (s, 2H), 6.86 (d, 1H), 7.29-7.38 (br m, 1H), 7.37 (s, 1H), 8.24 (d, 1H); MH+ 366.2.

### Example 91

### 1-[3-[[4-(2-Methyl-3-propan-2-yl-imidazol-4-yl)pyrimidin-2-yl]amino]pyrrolidin-1-yl]ethanone

Acetic anhydride (0.060 ml, 0.64 mmol) was added dropwise to a solution of 4-(2-methyl-3-propan-2-yl-imidazol-4-yl)-N-pyrrolidin-3-yl-pyrimidin-2-amine (Example 88; 100 mg, 0.35 mmol) and TEA (0.10 ml, 0.72 mmol) in DCM (3 ml). The reaction mixture was stirred for 2 hrs then water (5 ml) and DCM (5 ml) was added. The mixture was filtered through a PTFE filter cup, evaporated then purified by flash chromatography on silica, eluting with 10% MeOH in DCM to give a colourless gum. The gum was triturated with ether, filtered and dried to give the title compound as a colourless solid (79 mg, 69%). NMR (400.132 MHz) (rotamers) 1.49 (m, 6H), 1.88-2.03 (m, 4H), 2.05-2.24 (m, 1H), 2.48 (m, 3H), 3.43 (m, 2H), 3.56-3.75 (m, 2H), 4.31-4.44 (m, 1H), 5.66 (m, 1H), 6.86 (m, 1H), 7.34-7.49 (m, 2H), 8.24 (m, 1H); MH+ 329.3.

### Example 92

### N-[1-(3-Chloropropylsulphonyl)pyrrolidin-3-yl]-4-(2-methyl-3-propan-2-yl-imidazol-4-yl)pyrimidin-2-amine

The title compound was prepared by the procedure of Example 3 and on the same scale, using 4-(2-methyl-3-propan-2-yl-imidazol-4-yl)-N-pyrrolidin-3-yl-pyrimidin-2-amine (Example 88) as the starting material. NMR (400.132 MHz) 1.55 (d, 6H), 2.06 (m, 1H), 2.18 (m, 2H), 2.28 (m, 1H), 2.54 (s, 3H), 3.25-3.45 (m, 4H), 3.56 (m, 1H), 3.65 (m, 1H), 3.79 (t, 2H), 4.47 (m, 1H), 5.69 (m, 1H), 6.93 (d, 1H), 7.42 (s, 1H), 7.48 (m, 1H), 8.30 (d, 1H); MH+ 427.

### Examples 93-98

The following compounds were prepared by the procedure of Example 68 and on the same scale, using 4-(2-methyl-3-propan-2-yl-imidazol-4-yl)-N-pyrrolidin-3-yl-pyrimidin-2-amine (Example 88) and the appropriate acid starting material.

| Ex | Compound | NMR (500.133 MHz) | m/z |
|---|---|---|---|
| 93 | tert-Butyl 4-[2-[3-[[4-(2- | (373K) 1.09 (m, 2H), 1.40 (s, 9H), 1.51 (d, | 512.4 |
| | methyl-3-propan-2-yl- | 6H), 1.67 (m, 2H), 1.87-2.06 (m, 2H), | |
| | imidazol-4-yl)pyrimidin-2- | 2.17 (m, 3H), 2.49 (s, 3H), 2.74 (m, 2H), | |
| | yl]amino]pyrrolidin-1-yl]-2- | 3.28-3.77 (m, 4H), 3.88 (m, 2H), 4.43 (m, 1H), | |
| | oxo-ethyl]piperidine-1- | 5.58 (m, 1H), 6.82 (d, 1H), 6.94 (m, 1H), | |
| | carboxylate | 7.33 (s, 1H), 8.23 (d, 1H) | |
| 94 | tert-Butyl 4-[3-[3-[[4-(2- | (373K) 1.41 (s, 9H), 1.50 (d, 6H), | 527.3 |
| | methyl-3-propan-2-yl- | 1.89-2.26 (m, 2H), 2.32-2.43 (m, 6H), 2.47 (s, | |
| | imidazol-4-yl)pyrimidin-2- | 3H), 2.61 (m, 2H), 3.25-3.83 (m, 8H), | |
| | yl]amino]pyrrolidin-1-yl]-3- | 4.43 (m, 1H), 5.58 (m, 1H), 6.81 (d, 1H), 6.91 (s, | |
| | oxo-propyl]piperazine-1- | 1H), 7.29 (s, 1H), 8.22 (d, 1H) | |
| | carboxylate | | |
| 95 | tert-Butyl 4-[3-[3-[[4-(2- | (373K) 1.01 (m, 2H), 1.40 (s, 9H), | 526.4 |
| | methyl-3-propan-2-yl- | 1.42-1.54 (m, 9H), 1.63 (m, 2H), 1.89-2.28 (m, | |
| | imidazol-4-yl)pyrimidin-2- | 4H), 2.48 (s, 3H), 2.70 (m, 2H), | |
| | yl]amino]pyrrolidin-1-yl]-3- | 3.27-3.73 (m, 4H), 3.89 (m, 2H), 4.43 (m, 1H), | |
| | oxo-propyl]piperidine-1- | 5.58 (m, 1H), 6.81 (d, 1H), 6.93 (m, 1H), 7.32 (s, | |
| | carboxylate | 1H), 8.23 (d, 1H) | |
| 96 | tert-Butyl 4-methyl-4-[3- | (373K) 1.18 (s, 3H), 1.36 (m, 2H), 1.40 (s, | 512.6 |
| | [[4-(2-methyl-3-propan-2- | 9H), 1.51 (d, 6H), 1.94 (m, 1H), 2.06 (m, | |
| | yl-imidazol-4-yl)pyrimidin- | 2H), 2.15 (m, 1H), 2.49 (s, 3H), 3.15 (m, | |
| | 2-yl]amino]pyrrolidine-1- | 2H), 3.44-3.58 (m, 4H), 3.69-3.82 (m, | |
| | carbonyl]piperidine-1- | 2H), 4.41 (m, 1H), 5.57 (m, 1H), 6.82 (d, | |
| | carboxylate | 1H), 6.93 (m, 1H), 7.33 (s, 1H), 8.23 (d, 1H) | |
| 97 | tert-Butyl (3R)-3-[2-[3-[[4- | 1.21 (m, 1H), 1.32-1.43 (m, 10H), 1.51 (d, | 512.6 |
| | (2-methyl-3-propan-2-yl- | 6H), 1.57 (m, 1H), 1.78 (m, 1H), | |
| | imidazol-4-yl)pyrimidin-2- | 1.84-2.29 (m, 5H), 2.48 (s, 3H), 2.67 (m, 1H), 2.85 (m, | |
| | yl]amino]pyrrolidin-1-yl]-2- | 1H), 3.27-3.83 (m, 6H), 4.43 (m, 1H), | |
| | oxo-ethyl]piperidine-1- | 5.58 (m, 1H), 6.81 (d, 1H), 6.94 (m, 1H), 7.32 (s, | |
| | carboxylate | 1H), 8.23 (d, 1H) | |
| 98 | tert-Butyl (3S)-3-[2-[3-[[4- | (373K) 1.21 (m, 1H), 1.33-1.43 (m, 10H), | 512.6 |
| | (2-methyl-3-propan-2-yl- | 1.51 (d, 6H), 1.57 (m, 1H), 1.78 (m, 1H), | |
| | imidazol-4-yl)pyrimidin-2- | 1.84-2.29 (m, 5H), 2.49 (s, 3H), 2.67 (m, | |
| | yl]amino]pyrrolidin-1-yl]-2- | 1H), 2.85 (m, 1H), 3.28-3.83 (m, 6H), | |
| | oxo-ethyl]piperidine-1- | 4.43 (m, 1H), 5.58 (m, 1H), 6.82 (d, 1H), | |
| | carboxylate | 6.94 (m, 1H), 7.33 (s, 1H), 8.23 (d, 1H) | |

### Examples 99-104

The following compounds were prepared by the procedure of Example 80 and on the same scale using the indicated starting material in place of tert-butyl 4-[2-[4-[[4-(2-methyl-3-propan-2-yl-imidazol-4-yl)pyrimidin-2-yl]amino]-1-piperidinyl]-2-oxo-ethyl]piperidine-1-carboxylate (Example 68).

| Ex | Compound | NMR (500.133 MHz) | m/z | SM |
|---|---|---|---|---|
| 99 | 1-[3-[[4-(2-Methyl-3- | (500.133 MHz) 0.95-1.10 (m, 2H), | 412.3 | Example |
| | propan-2-yl-imidazol- | 1.47 (m, 6H), 1.58 (m, 2H), 1.76 (m, | | 93 |
| | 4-yl)pyrimidin-2- | 1H), 1.87-2.00 (m, 1H), | | |
| | yl]amino]pyrrolidin-1- | 2.04-2.21 (m, 3H), 2.38-2.48 (m, 5H), 2.87 (m, | | |
| | yl]-2-(4-piperidinyl)ethanone | 2H), 3.34-3.53 (m, 4H), | | |
| | | 4.26-4.42 (m, 1H), 5.65 (m, 1H), 6.84 (m, 1H), | | |
| | | 7.30-7.49 (m, 2H), 8.22 (m, 1H) | | |
| 100 | 1-[3-[[4-(2-Methyl-3- | 1.48 (m, 6H), 1.87-2.01 (m, 1H), | 427.2 | Example |
| | propan-2-yl-imidazol- | 2.02-2.22 (m, 1H), 2.22-2.40 (m, | | 94 |
| | 4-yl)pyrimidin-2- | 6H), 2.44-2.52 (m, 5H), 2.63 (m, | | |
| | yl]amino]pyrrolidin-1- | 2H), 2.67 (m, 2H), 3.24-3.70 (m, | | |
| | yl]-3-piperazin-1-yl- | 4H), 4.27-4.43 (m, 1H), 5.65 (m, | | |
| | propan-1-one | 1H), 6.84 (m, 1H), 7.31-7.49 (m, | | |
| | | 2H), 8.22 (m, 1H) | | |
| 101 | 1-[3-[[4-(2-Methyl-3- | 0.97 (m, 2H), 1.24-1.33 (m, 1H), | 426.3 | Example |
| | propan-2-yl-imidazol- | 1.35-1.61 (m, 10H), 1.87-2.24 (m, | | 95 |
| | 4-yl)pyrimidin-2- | 4H), 2.34-2.47 (m, 5H), 2.89 (m, | | |
| | yl]amino]pyrrolidin-1- | 2H), 3.22-3.73 (m, 4H), | | |
| | yl]-3-(4-piperidinyl)propan- | 4.26-4.43 (m, 1H), 5.65 (m, 1H), 6.84 (m, 1H), | | |
| | 1-one | 7.31-7.48 (m, 2H), 8.22 (m, 1H) | | |
| 102 | (4-Methyl-4- | (373K) 1.15 (s, 3H), 1.35 (m, 2H), | 412.4 | Example |
| | piperidinyl)-[3-[[4-(2- | 1.50 (m, 6H), 1.93 (m, 1H), 2.02 (m, | | 96 |
| | methyl-3-propan-2-yl- | 2H), 2.15 (m, 1H), 2.47 (s, 3H), | | |
| | imidazol-4- | 2.64-2.78 (m, 4H), 3.46-3.56 (m, 2H), | | |
| | yl)pyrimidin-2- | 3.72 (m, 1H), 3.80 (m, 1H), 4.39 (m, | | |
| | yl]amino]pyrrolidin-1- | 1H), 5.58 (m, 1H), 6.81 (d, 1H), | | |
| | yl]methanone | 6.88 (m, 1H), 7.29 (s, 1H), 8.22 (d, 1H) | | |
| 103 | 1-[3-[[4-(2-Methyl-3- | 1.04 (m, 1H), 1.33 (m, 1H), | 412.4 | Example |
| | propan-2-yl-imidazol- | 1.44-1.55 (m, 7H), 1.66-2.22 (m, 7H), | | 97 |
| | 4-yl)pyrimidin-2- | 2.40 (m, 1H), 2.46 (s, 3H), | | |
| | yl]amino]pyrrolidin-1- | 2.78-2.94 (m, 2H), 3.25-3.54 (m, 4H), | | |
| | yl]-2-[(3S)-3- | 4.27-4.43 (m, 1H), 5.65 (m, 1H), 6.84 (m, | | |
| | piperidinyl]ethanone | 1H), 7.33-7.49 (m, 2H), 8.22 (m, 1H) | | |
| 104 | 1-[3-[[4-(2-Methyl-3- | 1.04 (m, 1H), 1.33 (m, 1H), | 412.4 | Example |
| | propan-2-yl-imidazol- | 1.44-1.55 (m, 7H), 1.66-2.11 (m, 7H), | | 98 |
| | 4-yl)pyrimidin-2- | 2.39 (m, 1H), 2.46 (s, 3H), | | |
| | yl]amino]pyrrolidin-1- | 2.77-2.93 (m, 2H), 3.23-3.73 (m, 4H), | | |
| | yl]-2-[(3R)-3- | 4.26-4.43 (m, 1H), 5.65 (m, 1H), 6.84 (m, | | |
| | piperidinyl]ethanone | 1H), 7.32-7.48 (m, 2H), 8.22 (m, 1H) | | |

### Example 105

### 4-(2-Methyl-3-propan-2-yl-imidazol-4-yl)-N-[1-(3-pyrrolidin-1-ylpropylsulphonyl)pyrrolidin-3-yl]pyrimidin-2-amine

3-Chloro-1-propylsulfonyl chloride (0.046 ml, 0.38 mmol) was added dropwise to a solution of 4-(2-methyl-3-propan-2-yl-imidazol-4-yl)-N-pyrrolidin-3-yl-pyrimidin-2-amine (Example 88; 73 mg, 0.25 mmol) and TEA (0.070 ml, 0.50 mmol) in DCM (2 ml). The reaction mixture was stirred for 3 hrs then water (5 ml) and DCM (5 ml) were added and stirred for 5 mins. The organic layer was separated then evaporated to give an off white solid which was dissolved in THF (3 ml) then sodium iodide (5 mg, 0.03 mmol) and pyrrolidine (0.10 ml, 1.20 mmol) were added. The reaction mixture was heated by microwave at 150° C. for 1 hr then cooled and evaporated. The residue was purified by RPHPLC to give the title compound as a colourless solid (17 mg, 15%). NMR (400.132 MHz, CDCl₃) 1.56 (d, 6H), 1.85 (m, 4H), 2.00-2.15 (m, 3H), 2.32 (m, 1H), 2.58 (s, 3H), 2.64-2.78 (m, 6H), 3.11 (m, 2H), 3.36 (m, 1H), 3.55 (m, 2H), 3.72 (m, 1H), 4.56 (m, 1H), 5.58 (m, 1H), 5.68-5.93 (m, 1H), 6.79 (d, 1H), 7.34 (s, 1H), 8.19 (d, 1H); MH+ 462.3.

### Example 106

### 4-(2-Methyl-3-propan-2-yl-imidazol-4-yl)-N-[1-[3-(4-propan-2-ylpiperazin-1-yl)propylsulphonyl]pyrrolidin-3-yl]pyrimidin-2-amine

Sodium iodide (5 mg, 0.03 mmol) and 1-isopropylpiperazine (116 mg, 0.90 mmol) were added to a solution of N-[1-(3-chloropropylsulphonyl)pyrrolidin-3-yl]-4-(2-methyl-3-propan-2-yl-imidazol-4-yl)pyrimidin-2-amine (Example 92; 75 mg, 0.18 mmol) in THF (3 ml). The reaction mixture was heated by microwave at 150° C. for 45 mins. The reaction mixture was cooled, evaporated and the residue obtained purified by RPHPLC to give the title compound as an off-white solid (49 mg, 52%). NMR (400.132 MHz, CDCl₃) 1.33 (d, 6H), 1.59 (t, 6H), 2.05 (m, 3H), 2.35 (m, 1H), 2.61 (t, 2H), 2.65 (s, 3H), 2.79-2.97 (m, 8H), 3.07 (m, 2H), 3.40 (m, 2H), 3.56 (m, 2H), 3.72 (m, 1H), 4.58 (m, 1H), 5.57 (m, 1H), 5.78 (m, 1H), 6.83 (d, 1H), 7.43 (s, 1H), 8.25 (d, 1H); MH+ 519.3.

### Examples 107-108

The following compounds were prepared by the procedure of Example 106 and on the same scale by using the appropriate amine.

| Ex | Compound | NMR (400.132 MHz, CDCl₃) | m/z |
|---|---|---|---|
| 107 | N-[1-[3-(2-Methoxyethylamino)- | 1.56 (d, 6H), 2.08-2.37 (m, 4H), | 466.3 |
| | propylsulphonyl]pyrrolidin-3- | 2.58 (s, 3H), 3.03 (m, 4H), 3.11-3.72 (m, | |
| | yl]-4-(2-methyl-3-propan-2-yl- | 8H), 3.37 (s, 3H), 4.54 (m, 1H), | |
| | imidazol-4-yl)pyrimidin-2- | 5.59 (m, 1H), 6.08 (m, 1H), 6.80 (d, 1H), | |
| | amine | 7.36 (s, 1H), 8.19 (d, 1H) | |
| 108 | N-[1-[3-(4-Methylpiperazin-1- | 1.56 (d, 6H), 2.02 (m, 3H), 2.32 (m, | 491.3 |
| | yl)propylsulphonyl]pyrrolidin-3- | 1H), 2.39 (s, 3H), 2.50 (t, 2H), | |
| | yl]-4-(2-methyl-3-propan-2-yl- | 2.53-2.69 (m, 11H), 3.07 (m, 2H), 3.35 (m, | |
| | imidazol-4-yl)pyrimidin-2- | 1H), 3.55 (m, 2H), 3.72 (m, 1H), | |
| | amine | 4.56 (m, 1H), 5.58 (m, 1H), 5.72 (m, 1H), | |
| | | 6.80 (d, 1H), 7.35 (s, 1H), 8.19 (d, 1H) | |

### Example 109

### N-Methyl-3-[[4-(2-methyl-3-propan-2-yl-imidazol-4-yl)pyrimidin-2-yl]amino]pyrrolidine-1-carboxamide

4-(2-Methyl-3-propan-2-yl-imidazol-4-yl)-N-pyrrolidin-3-yl-pyrimidin-2-amine (Example 88; 144 mg, 0.5 mmol) was dissolved in a solution of methyl isocyanate (105 mg) in THF (2 ml) and stirred for 2 hrs at ambient temperature. Trisamine resin (200 mg) was added, the reaction mixture stirred gently for 30 mins then filtered and evaporated in vacuo to give a colourless gum. DCM (0.5 ml)/ether (3 ml) was added and the solution concentrated in vacuo to give the title compound as a colourless foam (51 mg, 30%). NMR (CDCl₃) 1.51 (d, 6H), 1.95-2.05 (m, 1H), 2.13-2.24 (m, 1H), 2.58 (s, 3H), 2.74 (s, 3H), 3.37-3.42 (m, 2H), 3.47-3.61 (m, 2H), 5.47-5.66 (m, 1H), 6.73 (d, 1H), 7.43 (s, 1H), 8.04 (d, 1H).

### Example 110

### N-(2-Dimethylaminoethyl)-3-[[4-(2-methyl-3-propan-2-yl-imidazol-4-yl)pyrimidin-2-yl]amino]pyrrolidine-1-carboxamide

The title compound was prepared in a similar manner to Example 22 and on a similar scale by using 4-(2-methyl-3-propan-2-yl-imidazol-4-yl)-N-pyrrolidin-3-yl-pyrimidin-2-amine (Example 88) in place of 4-(2-methyl-3-propan-2-yl-imidazol-4-yl)-N-(4-piperidinyl)pyrimidin-2-amine (Example 2) with the appropriate amine. NMR (CDCl₃, 400 MHz) 1.56 (d, 6H), 1.95-2.07 (m, 1H), 2.19-2.32 (m, 7H), 2.43 (t, 2H), 2.57 (s, 3H), 3.28-3.41 (m, 3H), 3.43-3.60 (m, 2H), 3.66-3.76 (m, 1H), 4.49-4.60 (m, 1H), 4.89 (s, 1H), 5.15 (d, 1H), 5.52-5.67 (m, 1H), 6.78 (d, 1H), 7.32 (s, 1H), 8.21 (d, 1H); MH+ 401.6.

### Example 111

### tert-Butyl(3S)-3-[[4-(2-methyl-3-propan-2-yl-imidazol-4-yl)pyrimidin-2-yl]amino]piperidine-1-carboxylate

2-Chloro-4-(2-methyl-3-propan-2-yl-imidazol-4-yl)pyrimidine (Method 5; 5.0 g, 21.2 mmol), TEA (6.5 ml, 46.6 mmol) and tert-butyl (3S)-3-aminopiperidine-1-carboxylate (4.33 g, 14.2 mmol) were added to DMA (100 ml) and heated at 110° C. for 16 hrs. The solvent was evaporated to give a yellow gum, water (100 ml) was added and the mixture was then extracted with DCM (3×150 ml). The combined organics were dried and solvent removed in vacuo to yield a dark gum (7.0 g). MH+ 401.

### Example 112

### 4-(2-Methyl-3-propan-2-yl-imidazol-4-yl)-N-[(3S)-3-piperidinyl]pyrimidin-2-amine

tert-Butyl (3S)-3-[[4-(2-methyl-3-propan-2-yl-imidazol-4-yl)pyrimidin-2-yl]amino]piperidine-1-carboxylate (Example 111; 5.5 g, 12.9 mmol) was dissolved in acetonitrile (30 ml) and 6.0M HCl in propan-2-ol (50 ml) added. The reaction was stirred for 2 hrs then evaporated to dryness, the gum obtained was dissolved in water (100 ml) and solid NaHCO₃ added until the reaction was basic. The aqueous layer was extracted with DCM (3×200 ml), dried and solvent removed in vacuo to give a yellow gum. Purification by flash chromatography on silica, eluting with 0-10% MeOH in DCM, gave the title compound as a dark gum (2.5 g). NMR (400.132 MHz, CDCl₃) 1.50-1.62 (m, 8H), 1.74-1.82 (m, 1H), 1.92-2.00 (m, 1H), 2.56 (s, 3H), 2.68 (dd, 1H), 2.72-2.78 (m, 1H), 2.87-2.92 (m, 1H), 3.18-3.22 (m, 1H), 3.93-3.96 (m, 1H), 5.39 (d, 1H), 5.55-5.67 (m, 1H), 6.71 (d, 1H), 7.30 (s, 1H), 8.19 (d, 1H); MH+ 301.

### Example 113

tert-Butyl (3R)-3-[[4-(2-methyl-3-propan-2-yl-imidazol-4-yl)pyrimidin-2-yl]amino]piperidine-1-carboxylate

The title compound was prepared in a similar manner to Example 111 and on a similar scale by using tert-butyl (3R)-3-aminopiperidine-1-carboxylate as the starting material. MH+ 401.

### Example 114

### 4-(2-Methyl-3-propan-2-yl-imidazol-4-yl)-N-[(3R)-3-piperidinyl]pyrimidin-2-amine

The title compound was prepared in a similar manner to Example 112 and on a similar scale by using tert-butyl (3R)-3-[[4-(2-methyl-3-propan-2-yl-imidazol-4-yl)pyrimidin-2-yl]amino]piperidine-1-carboxylate (Example 113) as the starting material. NMR (400.132 MHz, CDCl₃) 1.51-1.57 (m, 6H), 1.62-1.71 (m, 2H), 1.85-2.00 (m, 2H), 2.56 (s, 3H), 2.81-3.00 (m, 3H), 3.24-3.28 (m, 1H), 3.34-3.52 (m, 1H), 4.02-4.13 (m, 1H), 5.58-5.65 (m, 2H), 6.72 (d, 1H), 7.31 (s, 1H), 8.19 (d, 1H); MH+ 301.

### Example 115

### 4-(2-Methyl-3-propan-2-yl-imidazol-4-yl)-N-[(3S)-1-methylsulfonyl-3-piperidinyl]pyrimidin-2-amine

4-(2-Methyl-3-propan-2-yl-imidazol-4-yl)-N-[(3S)-3-piperidinyl]pyrimidin-2-amine (Example 112; 0.30 g, 0.1 mmol) and TEA (0.21 ml, 1.5 mmol) were added to DCM (10 ml) and then mesyl chloride (0.091 ml, 1.2 mmol) was added. After stirring for 10 mins sat. aq. NaHCO₃ (30 ml) was added, the mixture extracted with DCM (3×30 ml), dried and the solvent removed in vacuo to give a light yellow gum. Purification by flash chromatography on silica, eluting with 0-5% MeOH in DCM, gave the title compound as a white solid (0.26 g). NMR (400.132 MHz, CDCl₃) 1.56-1.59 (m, 7H), 1.69-1.78 (m, 1H), 1.89-2.07 (m, 2H), 2.57 (s, 3H), 2.78 (s, 3H), 2.84-2.92 (m, 1H), 2.95-3.09 (m, 1H), 3.37-3.46 (m, 1H), 3.69-3.72 (m, 1H), 4.15-4.23 (m, 1H), 5.25 (d, 1H), 5.57 (septet, 1H), 6.77 (d, 1H), 7.32 (s, 1H), 8.21 (d, 1H); MH+ 379.

### Example 116

### 4-(2-Methyl-3-propan-2-yl-imidazol-4-yl)-N-[(3R)-1-methylsulfonyl-3-piperidinyl]pyrimidin-2-amine

The title compound was prepared in a similar manner to Example 115 and on a similar scale by using 4-(2-methyl-3-propan-2-yl-imidazol-4-yl)-N-[(3R)-3-piperidinyl]pyrimidin-2-amine (Example 114) as the starting material. NMR (400.132 MHz, CDCl₃) 1.56-1.66 (m, 7H), 1.69-1.78 (m, 1H), 1.88-2.00 (m, 2H), 2.57 (s, 3H), 2.78 (s, 3H), 2.83-2.94 (m, 1H), 2.95-3.08 (m, 1H), 3.37-3.46 (m, 1H), 3.69-3.72 (m, 1H), 4.15-4.23 (m, 1H), 5.27 (d, 1H), 5.56 (septet, 1H), 6.77 (d, 1H), 7.32 (s, 1H), 8.20 (d, 1H); MH+ 379.

### Example 117

### tert-Butyl 4-[[5-chloro-4-(2-methyl-3-propan-2-yl-imidazol-4-yl)pyrimidin-2-yl]amino]piperidine-1-carboxylate

2,5-Dichloro-4-(2-methyl-3-propan-2-yl-imidazol-4-yl)pyrimidine (Method 6; 3.5 g, 12.9 mmol), TEA (3.95 ml, 28.4 mmol) and tert-butyl 4-aminopiperidine-1-carboxylate (2.84 g, 14.2 mmol) were added to DMA (80 ml) and heated at 100° C. for 16 hrs. The solvent was evaporated to dryness to give a yellow gum, water was added (100 ml), the reaction was then extracted with DCM (3×150 ml), combined organics dried and the solvent removed in vacuo to give the title compound as a yellow solid (5.5 g). MH+ 435.

### Example 118

### 5-Chloro-4-(2-methyl-3-propan-2-yl-imidazol-4-yl)-N-(4-piperidinyl)pyrimidin-2-amine

tert-Butyl 4-[[5-chloro-4-(2-methyl-3-propan-2-yl-imidazol-4-yl)pyrimidin-2-yl]amino]piperidine-1-carboxylate (Example 117; 5.5 g, 12.9 mmol) was dissolved in acetonitrile (30 ml) and 6.0 N HCl in propan-2-ol (50 ml) added. The reaction was stirred for 2 hrs, then evaporated to dryness. The residue obtained was dissolved in water (100 ml) and solid NaHCO₃ added until the reaction was basic (pH 9). The aqueous layer was then extracted with DCM (3×200 ml), the combined organics dried and solvent removed in vacuo to yield a yellow solid. This was dissolved in a minimum amount of hot acetonitrile and cooled to precipitate a solid which was filtered. The filtrate was concentrated and the process repeated to obtain two additional batches which were combined to give the title compound as a colourless solid (3.7 g, 85%). NMR (400.132 MHz, CDCl₃) 1.40 (ddd, 2H), 1.53 (d, 6H), 2.00-2.08 (m, 2H), 2.58 (s, 3H), 2.67-2.73 (m, 2H), 3.11 (ddd, 2H), 3.82-3.92 (m, 1H), 4.89-5.10 (m, 2H), 7.48 (s, 1H), 8.26 (s, 1H); MH+ 337.

### Example 119

### 5-Chloro-4-(2-methyl-3-propan-2-yl-imidazol-4-yl)-N-(1-methylsulphonyl-4-piperidinyl)pyrimidin-2-amine

The title compound was prepared in a similar manner to Example 115 and on a similar scale by using 5-chloro-4-(2-methyl-3-propan-2-yl-imidazol-4-yl)-N-(4-piperidinyl)pyrimidin-2-amine (Example 118) as the starting material. NMR (400.132 MHz, CDCl₃) 1.52 (d, 6H), 1.64 (ddd, 2H), 2.13-2.17 (m, 2H), 2.58 (s, 3H), 2.81 (s, 3H), 2.85-2.92 (m, 2H), 3.75-3.78 (m, 2H), 3.87-3.97 (m, 1H), 4.90 (septet, 1H), 5.12 (d, 1H), 7.45 (s, 1H), 8.29 (s, 1H); MH+ 415.

### Example 120

### 5-Chloro-4-(2-methyl-3-propan-2-yl-imidazol-4-yl)-N-[1-(2-morpholin-4-ylethylsulphonyl)-4-piperidinyl]pyrimidin-2-amine

5-Chloro-4-(2-methyl-3-propan-2-yl-imidazol-4-yl)-N-(4-piperidinyl)pyrimidin-2-amine (Example 118; 0.2 g, 0.6 mmol) and TEA (0.12 ml, 0.90 mmol) were dissolved in DCM (15 ml) and cooled to −10° C. 2-Chloroethane sulfonyl chloride (0.011 g, 0.66 mmol) was slowly added and the reaction was allowed to warm up to ambient temperature and stirred for 30 mins before adding morpholine (0.2 ml). After stirring for 16 hrs the reaction mixture was evaporated to dryness then purified by passing through a SCX column followed by RPHPLC to obtain the title compound as a colourless gum (146 mg). NMR (400.132 MHz, CDCl₃) 1.52 (d, 6H), 1.57-1.66 (m, 2H), 2.10-2.17 (m, 2H), 2.27 (s, 6H), 2.58 (s, 3H), 2.74-2.77 (m, 2H), 2.94-3.01 (m, 2H), 3.08-3.12 (m, 2H), 3.76-3.79 (m, 2H), 3.89-3.97 (m, 1H), 4.90-4.93 (m, 1H), 5.16 (s, 1H), 7.46 (s, 1H), 8.28 (s, 1H); MH+ 514.

### Examples 121-123

The following compounds were prepared by the procedure of Example 120 and on the same scale by using the appropriate amine.

| Ex | Compound | NMR (400.132 MHz, CDCl₃) | m/z |
|---|---|---|---|
| 121 | 5-Chloro-N-[1-[2-(4- | 0.93 (d, 3H), 1.17-1.27 (m, 2H), | 526 |
| | methyl-1-piperidinyl)ethylsulphonyl]- | 1.32-1.43 (m, 1H), 1.52 (d, 6H), 1.56-1.67 (m, 4H), | |
| | 4- | 1.98-2.17 (m, 4H), 2.58 (s, 3H), | |
| | piperidinyl]-4-(2-methyl-3- | 2.78-2.86 (m, 4H), 2.94-3.01 (m, 2H), 3.12-3.16 (m, | |
| | propan-2-yl-imidazol-4- | 2H), 3.75-3.78 (m, 2H), 3.87-3.96 (m, 1H), | |
| | yl)pyrimidin-2-amine | 4.91 (septet, 1H), 5.07 (d, 1H), 7.46 (s, | |
| | | 1H), 8.28 (s, 1H) | |
| 122 | 5-Chloro-N-[1-(2- | 1.52 (d, 6H), 1.57-1.66 (m, 2H), | 473 |
| | dimethylaminoethyl- | 2.10-2.17 (m, 2H), 2.27 (s, 6H), 2.58 (s, 3H), | |
| | sulphonyl)-4-piperidinyl]-4- | 2.74-2.77 (m, 2H), 2.94-3.01 (m, 2H), | |
| | (2-methyl-3-propan-2-yl- | 3.08-3.12 (m, 2H), 3.76-3.79 (m, 2H), 3.89-3.97 (m, | |
| | imidazol-4-yl)pyrimidin-2- | 1H), 4.90-4.93 (m, 1H), 5.16 (s, 1H), | |
| | amine | 7.46 (s, 1H), 8.28 (s, 1H) | |
| 123 | 5-Chloro-4-(2-methyl-3- | 1.52 (d, 6H), 1.56-1.65 (m, 2H), | 498 |
| | propan-2-yl-imidazol-4-yl)- | 1.79-1.82 (m, 4H), 2.10-2.13 (m, 2H), 2.53-2.56 (m, | |
| | N-[1-(2-pyrrolidin-1- | 4H), 2.58 (s, 3H), 2.89-3.00 (m, 4H), | |
| | ylethylsulphonyl)-4- | 3.14-3.17 (m, 2H), 3.76-3.79 (m, 2H), | |
| | piperidinyl]pyrimidin-2- | 3.88-3.95 (m, 1H), 4.89-4.93 (m, 1H), 5.11-5.12 (m, | |
| | amine | 1H), 7.46 (s, 1H), 8.28 (s, 1H) | |

### Example 124

### 5-Chloro-N-[1-(3-methyl-3-nitro-butyl)sulfonyl-4-piperidinyl]-4-(2-methyl-3-propan-2-yl-imidazol-4-yl)pyrimidin-2-amine

5-Chloro-4-(2-methyl-3-propan-2-yl-imidazol-4-yl)-N-(4-piperidinyl)pyrimidin-2-amine (Example 118; 0.2 g, 0.60 mmol) and TEA (0.13 ml, 0.90 mmol) were dissolved in DCM (7 ml). 2-Chloroethane sulfonyl chloride (0.07 ml, 0.66 mmol) was slowly added and the mixture warmed to ambient temperature and stirred for 30 mins. The reaction mixture was evaporated to dryness then dissolved in DMA (7 ml) then 1,8-diazabicyclo[5.4.0]undec-7-ene (0.191 g, 1.20 mmol) and 2-nitropropane (0.112 g, 1.20 mmol) were added. The reaction was heated at 60° C. for 30 mins. Water was then added, the aqueous layer extracted with DCM (3×50 ml), dried and the solvent removed in vacuo. Purification by flash chromatography on silica, eluting with 0-5% MeOH in DCM, gave the title compound as a yellow gum. NMR (400.132 MHz, CDCl₃) 1.52 (d, 6H), 1.57-1.66 (m, 8H), 2.11-2.15 (m, 2H), 2.36-2.41 (m, 2H), 2.58 (s, 3H), 2.92-2.97 (m, 2H), 2.99-3.03 (m, 2H), 3.76-3.79 (m, 2H), 3.90-3.99 (m, 1H), 4.92 (septet, 1H), 5.31-5.37 (m, 1H), 7.45 (s, 1H), 8.28 (s, 1H); MH+ 516.

### Example 125

### 5-Fluoro-N-[1-(3-methyl-3-nitro-butyl)sulfonyl-4-piperidinyl]-4-(2-methyl-3-propan-2-yl-imidazol-4-yl)pyrimidin-2-amine

The title compound was prepared in a similar manner to Example 124 and on a similar scale by using 5-fluoro-4-(2-methyl-3-propan-2-yl-imidazol-4-yl)-N-(4-piperidinyl)pyrimidin-2-amine (Example 137) as the starting material. NMR (400.132 MHz, CDCl₃) 1.55-1.64 (m, 14H), 2.14-2.17 (m, 2H), 2.37-2.41 (m, 2H), 2.60 (s, 3H), 2.92-3.05 (m, 4H), 3.77-3.80 (m, 2H), 3.85-3.96 (m, 1H), 4.92 (d, 1H), 5.49 (septet, 1H), 7.53 (s, 1H), 8.16 (s, 1H); MH+ 498.

### Example 126

### N-[1-(3-Amino-3-methyl-butyl)sulfonyl-4-piperidinyl]-5-chloro-4-(2-methyl-3-propan-2-yl-imidazol-4-yl)pyrimidin-2-amine

5-Chloro-N-[1-(3-methyl-3-nitro-butyl)sulfonyl-4-piperidinyl]-4-(2-methyl-3-propan-2-yl-imidazol-4-yl)pyrimidin-2-amine (Example 124; 189 mg, 0.37 mmol) was dissolved in acetic acid (20 ml), to this was added iron (63 mg, 1.10 mmol) and the reaction was heated at 60° C. for 1 hr. After which the reaction was evaporated to dryness, quenched with 1.0M NaOH (50 ml), extracted with DCM (3×100 ml), combined organics dried and the solvent removed in vacuo to yield a yellow gum (0.16 g, 70%). MH+ 486.

### Example 127

### N-[1-(3-Amino-3-methyl-butyl)sulphonyl-4-piperidinyl]-5-fluoro-4-(2-methyl-3-propan-2-yl-imidazol-4-yl)pyrimidin-2-amine

The title compound was prepared in a similar manner to Example 126 and on a similar scale by using 5-fluoro-N-[1-(3-methyl-3-nitro-butyl)sulfonyl-4-piperidinyl]-4-(2-methyl-3-propan-2-yl-imidazol-4-yl)pyrimidin-2-amine (Example 125) as the starting material. NMR (400.132 MHz, CDCl₃) 1.16 (s, 6H), 1.45 (brs, 2H), 1.55-1.67 (m, 8H), 1.83-1.87 (m, 2H), 2.12-2.16 (m, 2H), 2.60 (s, 3H), 2.97-3.08 (m, 4H), 3.77-3.80 (m, 2H), 3.84-3.93 (m, 1H), 4.93 (d, 1H), 5.49 (septet, 1H), 7.52 (d, 1H), 8.15 (d, 1H); MH+ 468.

### Example 128

### 5-Chloro-N-[1-(3-dimethylamino-3-methyl-butyl)sulphonyl-4-piperidinyl]-4-(2-methyl-3-propan-2-yl-imidazol-4-yl)pyrimidin-2-amine

MeOH (15 ml) and formaldehyde (1.0 ml) were added to N-[1-(3-amino-5-methyl-butyl)sulfonyl-4-piperidinyl]-5-chloro-4-(2-methyl-3-propan-2-yl-imidazol-4-yl)pyrimidin-2-amine (Example 126; 160 mg, 0.33 mmol) then sodium cyanoborohydride (63 mg, 1.0 mmol) and the reaction was stirred for 30 mins before adding NaOH (20 ml). The reaction was extracted with DCM (3×50 ml), dried and the solvent removed in vacuo. Purification by flash chromatography on silica, eluting with 0-10% MeOH in DCM gave the title compound as an off-white foam (0.068 g, 44%). NMR (400.132 MHz, CDCl₃) 1.04 (s, 6H), 1.52 (d, 6H), 1.62 (ddd, 2H), 1.88-1.92 (m, 2H), 2.10-2.14 (m, 2H), 2.21 (s, 6H), 2.58 (s, 3H), 2.94-3.04 (m, 4H), 3.77 (d, 2H), 3.88-3.97 (m, 1H), 4.91 (septet, 1H), 5.12-5.19 (m, 1H), 7.45 (s, 1H), 8.28 (s, 1H); MH+ 514.

### Example 129

### N-[1-(3-Dimethylamino-3-methyl-butyl)sulphonyl-4-piperidinyl]-5-fluoro-4-(2-methyl-3-propan-2-yl-imidazol-4-yl)pyrimidin-2-amine

The title compound was prepared in a similar manner to Example 128 and on a similar scale by using N—[1-(3-amino-3-methyl-butyl)sulphonyl-4-piperidinyl]-5-fluoro-4-(2-methyl-3-propan-2-yl-imidazol-4-yl)pyrimidin-2-amine (Example 127) as the starting material. NMR (400.132 MHz, CDCl₃) 1.03 (s, 6H), 1.55-1.67 (m, 8H), 1.88-1.92 (m, 2H), 2.12-2.16 (m, 2H), 2.20 (s, 6H), 2.60 (s, 3H), 2.96-3.04 (m, 4H), 3.76-3.79 (m, 2H), 3.83-3.93 (m, 1H), 4.99 (d, 1H), 5.50 (septet, 1H), 7.52 (d, 1H), 8.15 (d, 1H); MH+ 496.

### Example 130

### 5-Chloro-N-[1-(3-dimethylaminopropylsulphonyl)-4-piperidinyl]-4-(2-methyl-3-propan-2-yl-imidazol-4-yl)pyrimidin-2-amine

5-Chloro-4-(2-methyl-3-propan-2-yl-imidazol-4-yl)-N-(4-piperidinyl)pyrimidin-2-amine (Example 118; 1.0 g, 2.99 mmol) and TEA (0.62 ml, 4.50 mmol) were dissolved in DCM (30 ml) and cooled to −10° C. To this was slowly added 2-chloropropane sulfonyl chloride (0.58 ml, 3.29 mmol), the reaction warmed to ambient temperature and stirred for 30mins. After which the reaction mixture was evaporated to dryness, DMA (50 ml) was added then dimethylamine solution in MeOH (1 ml) and the reaction heated at 90° C. for 16 hrs. The reaction was then evaporated to dryness and purified by RPHPLC to give the title compound as an off white foam. NMR (400.132 MHz, CDCl₃) 1.52 (d, 6H), 1.56-1.66 (m, 2H), 1.93-2.00 (m, 2H), 2.07-2.15 (m, 2H), 2.22 (s, 6H), 2.39 (t, 2H), 2.58 (s, 3H), 2.94-3.02 (m, 4H), 3.76-3.79 (m, 2H), 3.88-3.95 (m, 1H), 4.91 (septet, 1H), 5.30 (s, 1H), 7.44 (s, 1H), 8.28 (s, 1H); MH+ 486.

### Examples 131-134

The following compounds were prepared by the procedure of Example 130 and on the same scale by using the appropriate amine in place of dimethylamine.

| Ex | Compound | NMR (400.132 MHz, CDCl₃) | m/z |
|---|---|---|---|
| 131 | 5-Chloro-4-(2-methyl-3- | 1.52 (d, 6H), 1.61 (ddd, 2H), 1.76-1.79 (m, | 512 |
| | propan-2-yl-imidazol-4- | 4H), 1.96-2.04 (m, 2H), 2.08-2.15 (m, 2H), | |
| | yl)-N-[1-(3-pyrrolidin-1- | 2.48-2.51 (m, 4H), 2.54-2.58 (m, 5H), | |
| | ylpropylsulphonyl)-4- | 2.94-3.05 (m, 4H), 3.76-3.79 (m, 2H), | |
| | piperidinyl]pyrimidin-2- | 3.88-3.95 (m, 1H), 4.91 (septet, 1H), 5.23 (s, 1H), | |
| | amine | 7.45 (s, 1H), 8.28 (s, 1H) | |
| 132 | 5-Chloro-4-(2-methyl-3- | 1.42-1.46 (m, 2H), 1.51-1.66 (m, 10H), | 526 |
| | propan-2-yl-imidazol-4- | 1.93-2.00 (m, 2H), 2.09-2.13 (m, 2H), | |
| | yl)-N-[1-[3-(1-piperidinyl)propylsulphonyl]- | 2.35-2.42 (m, 8H), 2.58 (s, 3H), 2.93-3.02 (m, | |
| | 4- | 4H), 3.76-3.79 (m, 2H), 3.88-3.97 (m, 1H), | |
| | piperidinyl]pyrimidin-2- | 4.91 (septet, 1H), 5.26 (s, 1H), 7.45 (s, 1H), | |
| | amine | 8.28 (s, 1H) | |
| 133 | 5-Chloro-N-[1-[3-(4- | 1.52 (d, 6H), 1.61 (ddd, 2H), 1.94-2.01 (m, | 541 |
| | methylpiperazin-1- | 2H), 2.10-2.14 (m, 2H), 2.28 (s, 3H), | |
| | yl)propylsulphonyl]-4- | 2.41-2.53 (m, 10H), 2.58 (s, 3H), 2.93-3.02 (m, | |
| | piperidinyl]-4-(2-methyl- | 4H), 3.75-3.78 (m, 2H), 3.88-3.97 (m, 1H), | |
| | 3-propan-2-yl-imidazol-4- | 4.90 (septet, 1H), 5.23 (s, 1H), 7.44 (s, 1H), | |
| | yl)pyrimidin-2-amine | 8.28 (s, 1H) | |
| 134 | 5-Chloro-4-(2-methyl-3- | 1.52 (d, 6H), 1.61 (ddd, 2H), 1.95-2.02 (m, | 528 |
| | propan-2-yl-imidazol-4- | 2H), 2.08-2.16 (m, 2H), 2.42-2.47 (m, 6H), | |
| | yl)-N-[1-(3-morpholin-4- | 2.58 (s, 3H), 2.93-3.03 (m, 4H), 3.70 (m, | |
| | ylpropylsulphonyl)-4- | 4H), 3.76-3.79 (m, 2H), 3.89-3.96 (m, 1H), | |
| | piperidinyl]pyrimidin-2- | 4.90 (septet, 1H), 5.26 (s, 1H), 7.44 (s, 1H), | |
| | amine | 8.28 (s, 1H) | |

### Example 135

### Benzyl 4-[[5-fluoro-4-(2-methyl-3-propan-2-yl-imidazol-4-yl)pyrimidin-2-yl]amino]piperidine-1-carboxylate

2-Chloro-5-fluoro-4-(2-methyl-3-propan-2-yl-imidazol-4-yl)pyrimidine (Method 8; 10 g, 39.4 mmol), DIPEA (15.4 ml, 86.7 mmol) and benzyl 4-aminopiperidine-1-carboxylate (11.0 g, 47.2 mmol) were added to DMA (200 ml) and heated at 125° C. for 2 days. The reaction mixture was evaporated to dryness, water (100 ml) added and the reaction extracted with DCM (3×150 ml), dried and the solvent removed in vacuo to yield a yellow solid. This was dissolved in DCM, propan-2-ol added and then the DCM slowly evaporated to yield a solid. This was filtered and washed with ether, the process was repeated with the filtrate to get two additional batches which were combined to give the title compound as an off-white solid (13.75 g). NMR (400.132 MHz, CDCl₃) 1.38-1.48 (m, 2H), 1.55 (d, 6H), 2.03-2.06 (m, 2H), 2.60 (s, 3H), 3.00 (t, 2H), 3.85-3.94 (m, 1H), 4.13-4.16 (m, 2H), 4.90 (d, 1H), 5.14 (s, 2H), 5.54 (septet, 1H), 7.29-7.37 (m, 5H), 7.52 (d, 1H), 8.15 (d, 1H); MH+ 453.

### Example 136

### tert-Butyl 4-[[5-fluoro-4-(2-methyl-3-propan-2-yl-imidazol-4-yl)pyrimidin-2-yl]amino]piperidine-1-carboxylate

2-Chloro-5-fluoro-4-(2-methyl-3-propan-2-yl-imidazol-4-yl)pyrimidine (Method 8; 5.0 g, 21.7 mmol), TEA (6.03 ml, 43.3 mmol) and tert-butyl 4-aminopiperidine-1-carboxylate (4.33 g, 21.7 mmol) were added to DMA (80 ml) and heated at 110° C. for 2 days. The solvent was evaporated to dryness to give a yellow gum, water was added (100 ml) and the aqueous layer was extracted with DCM (3×150 ml). The combined organics were dried and the solvent removed in vacuo to give a yellow solid (7.2 g, 87%). MH+ 419.

### Example 137

### 5-Fluoro-4-(2-methyl-3-propan-2-yl-imidazol-4-yl)-N-(4-piperidinyl)pyrimidin-2-amine

Benzyl 4-[[5-fluoro-4-(2-methyl-3-propan-2-yl-imidazol-4-yl)pyrimidin-2-yl]amino]piperidine-1-carboxylate (Example 135; 13.75 g, 30.4 mmol) and 10% Pd/C (1.3 g) in EtOH (700 ml) were stirred at 25° C. under hydrogen at 5 bar pressure for 18 hrs. The reaction mixture was filtered through diatomaceous earth and the solvent removed in vacuo to yield a yellow solid. This was dissolved in hot acetonitrile, then cooled to precipitate a solid, which was filtered and dried. The process was repeated with the filtrate to obtain additional material, then the batches were combined to give the title compound as a colorless solid (7.5 g). NMR (400.132 MHz, CDCl₃) 1.41 (ddd, 2H), 1.56 (d, 6H), 2.04-2.06 (m, 2H), 2.60 (s, 3H), 2.71 (t, 2H), 3.00-3.15 (m, 2H), 3.78-3.87 (m, 1H), 4.95 (d, 1H), 5.59-5.62 (m, 1H), 7.53 (d, 1H), 8.14 (d, 1H); MH+ 319.

### Example 138

### 5-Fluoro-4-(2-methyl-3-propan-2-yl-imidazol-4-yl)-N-(1-methylsulphonyl-4-piperidinyl)pyrimidin-2-amine

### 1-Methylsulfonylpiperidin-4-amine (0.098 g, 0.55 mmol) was suspended in isopropanol (2 ml) then 2-chloro-5-fluoro-4-(2-methyl-3-propan-2-yl-imidazol-4-yl)pyrimidine (Method 8; 70 mg, 0.27 mmol), TEA (0.11 ml, 0.82 mmol), sodium iodide (12 mg, 0.08 mmol) were added and the mixture heated by microwave at 160° C. for 500 mins. The solvent was removed in vacuo and purified by flash chromatography on silica, eluting with a gradient of 0-5% MeOH/DCM. Fractions containing product were combined and evaporated to give a gum which was triturated with ether and then evaporated to give the title compound as a cream solid (52 mg, 48%). NMR (400.132 MHz, CDCl₃) 1.48 (d, 6H), 1.58 (m, 2H), 2.10 (m, 2H), 2.53 (s, 3H), 2.75 (s, 3H), 2.84 (m, 2H), 3.70 (m, 2H), 3.81 (m, 1H), 4.81 (d, 1H), 5.41 (m, 1H), 7.45 (d, 1H), 8.09 (d, 1H); MH+ 397.

### Example 139

### N-[1-(2-Dimethylaminoethylsulphonyl)-4-piperidinyl]-5-fluoro-4-(2-methyl-3-propan-2-yl-imidazol-4-yl)pyrimidin-2-amine

5-Fluoro-4-(2-methyl-3-propan-2-yl-imidazol-4-yl)-N-(4-piperidinyl)pyrimidin-2-amine (Example 137; 200 mg, 0.62 mmol) was dissolved in DCM, TEA (0.26 ml, 1.87 mmol) added and the reaction cooled to 0° C. 2-Chloroethane sulfonylchloride (0.08 ml, 0.75 mmol) was added dropwise to the cooled solution, the reaction stirred for 20 mins then dimethylamine in MeOH (1 ml) was added. After stirring for 1 hr the solvent was removed in vacuo and the residue purified by RPHPLC to give the title compound as a gum (82 mg). NMR (400.132 MHz, CDCl₃) 1.55-1.66 (m, 8H), 2.11-2.15 (m, 2H), 2.28 (s, 6H), 2.60 (s, 3H), 2.74-2.78 (m, 2H), 2.96-3.02 (m, 2H), 3.09-3.12 (m, 2H), 3.76-3.79 (m, 2H), 3.84-3.93 (m, 1H), 4.99 (d, 1H), 5.50 (septet, 1H), 7.52 (d, 1H), 8.15 (d, 1H); MH+ 454.

### Examples 140 to 142

The following compounds were prepared by the procedure of Example 139 and on the same scale by using the appropriate amine.

| Ex | Compound | NMR (400.132 MHz, CDCl₃) | m/z |
|---|---|---|---|
| 140 | 5-Fluoro-4-(2-methyl-3- | 1.55-1.66 (m, 8H), 1.77-1.83 (m, 4H), | 480 |
| | propan-2-yl-imidazol-4-yl)- | 2.11-2.15 (m, 2H), 2.52-2.58 (m, 4H), | |
| | N-[1-(2-pyrrolidin-1- | 2.60 (s, 3H), 2.90-2.94 (m, 2H), | |
| | ylethylsulphonyl)-4- | 2.96-3.02 (m, 2H), 3.14-3.18 (m, 2H), | |
| | piperidinyl]pyrimidin-2- | 3.76-3.79 (m, 2H), 3.83-3.92 (m, 1H), 4.94 (d, | |
| | amine | 1H), 5.49 (septet, 1H), 7.52 (d, 1H), | |
| | | 8.15 (d, 1H) | |
| 141 | N-[1-[2-(7- | 1.35 (d, 4H), 1.55-1.66 (m, 8H), | 506 |
| | Azabicyclo[2.2.1]hept-7- | 1.70-1.77 (m, 2H), 1.94 (s, 2H), 2.11-2.17 (m, | |
| | yl)ethylsulphonyl]-4- | 2H), 2.60 (s, 3H), 2.79-2.83 (m, 2H), | |
| | piperidinyl]-5-fluoro-4-(2- | 2.96-3.03 (m, 2H), 3.11-3.15 (m, 2H), | |
| | methyl-3-propan-2-yl- | 3.26-3.28 (m, 2H), 3.75-3.80 (m, 2H), | |
| | imidazol-4-yl)pyrimidin-2- | 3.82-3.91 (m, 1H), 4.96 (d, 1H), | |
| | amine | 5.49 (septet, 1H), 7.52 (d, 1H), 8.15 (d, 1H) | |
| 142 | N-[1-[2-(6- | 1.47-1.66 (m, 14H), 1.86-1.95 (m, 4H), | 520 |
| | Azabicyclo[2.2.2]oct-6- | 2.11-2.16 (m, 2H), 2.60 (s, 3H), | |
| | yl)ethylsulfonyl]-4- | 2.70-2.73 (m, 2H), 2.92-3.03 (m, 4H), | |
| | piperidinyl]-5-fluoro-4-(2- | 3.09-3.13 (m, 2H), 3.76-3.80 (m, 2H), | |
| | methyl-3-propan-2-yl- | 3.83-3.92 (m, 1H), 4.98 (d, 1H), 5.49 (septet, | |
| | imidazol-4-yl)pyrimidin-2- | 1H), 7.51 (d, 1H), 8.15 (d, 1H) | |
| | amine | | |

### Example 143

### N-[1-(3-Chloropropylsulphonyl)-4-piperidinyl]-5-fluoro-4-(2-methyl-3-propan-2-yl-imidazol-4-yl)pyrimidin-2-amine

5-Fluoro-4-(2-methyl-3-propan-2-yl-imidazol-4-yl)-N-(4-piperidinyl)pyrimidin-2-amine (Example 137; 1.0 g, 3.14 mmol) and TEA (0.66 ml, 4.71 mmol) were dissolved in DCM (30 ml) and cooled to −10° C. 2-Chloropropane sulfonyl chloride (0.66 ml, 4.71 mmol) was added and the reaction was allowed to warm up to ambient temperature and stirred for 30mins. The reaction was evaporated to dryness and purified by flash chromatography on silica, eluting with 0-5% MeOH in DCM, to give the title compound as an off-white solid. NMR (400.132 MHz, CDCl₃) 1.55-1.67 (m, 8H), 2.13-2.17 (m, 2H), 2.26-2.33 (m, 2H), 2.60 (s, 3H), 2.97-3.04 (m, 2H), 3.11 (t, 2H), 3.70 (t, 2H), 3.78-3.81 (m, 2H), 3.85-3.94 (m, 1H), 4.95 (d, 1H), 5.49 (septet, 1H), 7.52 (d, 1H), 8.15 (d, 1H); MH+ 459.

### Example 144

### N-[1-(3-Dimethylaminopropylsulphonyl)-4-piperidinyl]-5-fluoro-4-(2-methyl-3-propan-2imidazol-4-yl)pyrimidin-2-amine

Dimethylamine in MeOH (1.0 ml) was added to a solution of N-[1-(3-chloropropylsulphonyl)-4-piperidinyl]-5-fluoro-4-(2-methyl-3-propan-2-yl-imidazol-4-yl)pyrimidin-2-amine (Example 143; 0.14 g, 0.31 mmol) in DMA (10 ml) and heated at 90° C. for 16 hrs. The reaction was then evaporated to dryness and purified by RPHPLC to give the title compound as a gum (91 mg). NMR (400.132 MHz, CDCl₃) 1.55-1.67 (m, 8H), 1.94-2.01 (m, 2H), 2.11-2.15 (m, 2H), 2.22 (s, 6H), 2.39 (t, 2H), 2.60 (s, 3H), 2.95-3.02 (m, 4H), 3.76-3.79 (m, 2H), 3.83-3.92 (m, 1H), 5.06 (d, 1H), 5.50 (septet, 1H), 7.51 (d, 1H), 8.15 (d, 1H); MH+ 468.

### Examples 145 to 166

The following compounds were prepared by the procedure of Example 144 and on the same scale by using the appropriate amine.

| Ex | Compound | NMR (400.132 MHz, CDCl₃) | m/z |
|---|---|---|---|
| 145 | 5-Fluoro-4-(2-methyl-3- | 1.55-1.66 (m, 8H), 1.77-1.80 (m, 4H), | 494 |
| | propan-2-yl-imidazol-4- | 1.98-2.05 (m, 2H), 2.11-2.15 (m, 2H), | |
| | yl)-N-[1-(3-pyrrolidin-1- | 2.50-2.53 (m, 4H), 2.56-2.60 (m, 5H), 2.94-3.06 (m, | |
| | ylpropylsulphonyl)-4- | 4H), 3.77 (d, 2H), 3.83-3.92 (m, 1H), | |
| | piperidinyl]pyrimidin-2- | 5.05 (d, 1H), 5.50 (septet, 1H), 7.51 (d, 1H), | |
| | amine | 8.15 (d, 1H) | |
| 146 | 5-Fluoro-4-(2-methyl-3- | 1.41-1.46 (m, 2H), 1.52-1.66 (m, 12H), | 508 |
| | propan-2-yl-imidazol-4- | 1.94-2.01 (m, 2H), 2.11-2.15 (m, 2H), | |
| | yl)-N-[1-[3-(1-piperidinyl)propylsulphonyl]- | 2.32-2.42 (m, 6H), 2.60 (s, 3H), 2.94-3.03 (m, | |
| | 4- | 4H), 3.76-3.79 (m, 2H), 3.84-3.92 (m, 1H), | |
| | piperidinyl]pyrimidin-2- | 5.06 (d, 1H), 5.50 (septet, 1H), 7.51 (d, 1H), | |
| | amine | 8.15 (d, 1H) | |
| 147 | 5-Fluoro-N-[1-[3-(4- | 1.55-1.67 (m, 8H), 1.94-2.02 (m, 2H), | 523 |
| | methylpiperazin-1- | 2.11-2.15 (m, 2H), 2.29 (s, 3H), 2.44-2.56 (m, | |
| | yl)propylsulphonyl]-4- | 10H), 2.60 (s, 3H), 2.94-3.03 (m, 4H), | |
| | piperidinyl]-4-(2-methyl- | 3.75-3.78 (m, 2H), 3.84-3.93 (m, 1H), 5.09 (d, | |
| | 3-propan-2-yl-imidazol-4- | 1H), 5.49 (septet, 1H), 7.51 (d, 1H), 8.15 (d, | |
| | yl)pyrimidin-2-amine | 1H) | |
| 148 | 5-Fluoro-4-(2-methyl-3- | 1.55-1.67 (m, 8H), 1.95-2.02 (m, 2H), | 510 |
| | propan-2-yl-imidazol-4- | 2.12-2.16 (m, 2H), 2.42-2.47 (m, 6H), 2.60 (s, | |
| | yl)-N-[1-(3-morpholin-4- | 3H), 2.94-3.04 (m, 4H), 3.69-3.71 (m, 4H), | |
| | ylpropylsulphonyl)-4- | 3.75-3.79 (m, 2H), 3.84-3.93 (m, 1H), | |
| | piperidinyl]pyrimidin-2- | 5.08 (d, 1H) 5.48 (septet, 1H), 7.51 (d, 1H), | |
| | amine | 8.15 (d, 1H) | |
| 149 | N-[1-[3-(6- | 1.44-1.66 (m, 15H), 1.87-1.97 (m, 4H), | 534 |
| | Azabicyclo[2.2.2]oct-6- | 2.11-2.15 (m, 2H), 2.48-2.51 (m, 1H), | |
| | yl)propylsulfonyl]-4- | 2.58-2.61 (m, 5H), 2.68 (s, 2H), 3.07-2.95 (m, | |
| | piperidinyl]-5-fluoro-4-(2- | 4H), 3.76-3.79 (m, 2H), 3.84-3.93 (m, 1H), | |
| | methyl-3-propan-2-yl- | 4.98 (d, 1H), 5.50 (septet, 1H), 7.52 (d, 1H), | |
| | imidazol-4-yl)pyrimidin-2- | 8.15 (d, 1H) | |
| | amine | | |
| 150 | N-[1-[3-(7- | 1.28-1.29 (m, 5H), 1.55-1.71 (m, 11H), | 520 |
| | Azabicyclo[2.2.1]hept-7- | 1.92-1.99 (m, 2H), 2.11-2.15 (m, 2H), | |
| | yl)propylsulphonyl]-4- | 2.45 (t, 2H), 2.60 (s, 3H), 2.95-3.02 (m, 2H), | |
| | piperidinyl]-5-fluoro-4-(2- | 3.06-3.10 (m, 2H), 3.23-3.25 (m, 2H), | |
| | methyl-3-propan-2-yl- | 3.76-3.80 (m, 2H), 3.84-3.93 (m, 1H), 5.03 (d, | |
| | imidazol-4-yl)pyrimidin-2- | 1H), 5.50 (septet, 1H), 7.52 (d, 1H), 8.15 (d, | |
| | amine | 1H) | |
| 151 | N-[1-[3- | (DMSO) 0.18-0.22 (m, 2H), 0.34-0.38 (m, | 480 |
| | (Cyclopropylamino)propylsulphonyl]- | 2H), 1.49-1.51 (m, 7H), 1.52-1.59 (m, 2H), | |
| | 4-piperidinyl]- | 1.76-1.83 (m, 2H), 1.95 (d, 2H), | |
| | 5-fluoro-4-(2-methyl-3- | 2.01-2.05 (m, 1H), 2.49 (s, 3H), 2.66 (t, 2H), 2.88 (t, | |
| | propan-2-yl-imidazol-4- | 3H), 3.04-3.08 (m, 2H), 3.62 (s, 2H), 3.78 (t, | |
| | yl)pyrimidin-2-amine | 1H), 5.43 (s, 1H), 7.22 (s, 1H), 7.32 (d, 1H), | |
| | | 8.37 (d, 1H) | |
| 152 | N-[1-[3- | | 508 |
| | (Cyclopentylamino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |
| 153 | N-[1-[3- | (DMSO) 1.49-1.51 (m, 6H), 1.52-1.83 (m, | 494 |
| | (Cyclobutylamino)propylsulphonyl]- | 9H), 1.96 (d, 2H), 2.06-2.12 (m, 2H), | |
| | 4-piperidinyl]- | 2.50 (s, 3H), 2.90 (d, 2H), 3.05-3.09 (m, 2H), | |
| | 5-fluoro-4-(2-methyl-3- | 3.12 (t, 1H), 3.18-3.19 (m, 1H), 3.61 (d, | |
| | propan-2-yl-imidazol-4- | 2H), 3.79 (t, 1H), 5.43 (s, 1H), 7.23 (s, 1H), | |
| | yl)pyrimidin-2-amine | 7.32 (d, 1H), 8.36-8.37 (m, 1H) | |
| 154 | 5-Fluoro-4-(2-methyl-3- | | 482 |
| | propan-2-yl-imidazol-4- | | |
| | yl)-N-[1-[3-(propan-2- | | |
| | ylamino)propylsulphonyl]- | | |
| | 4-piperidinyl]pyrimidin-2- | | |
| | amine | | |
| 155 | 5-Fluoro-N-[1-[3-(methyl- | | 496 |
| | propan-2-yl- | | |
| | amino)propylsulphonyl]-4- | | |
| | piperidinyl]-4-(2-methyl- | | |
| | 3-propan-2-yl-imidazol-4- | | |
| | yl)pyrimidin-2-amine | | |
| 156 | 3-[3-[[4-[[5-Fluoro-4-(2- | | 507 |
| | methyl-3-propan-2-yl- | | |
| | imidazol-4-yl)pyrimidin-2- | | |
| | yl]amino]-1-piperidinyl]sulphonyl]propyl- | | |
| | methyl- | | |
| | amino]propanenitrile | | |
| 157 | N-[1-[3-(Azetidin-1- | | 480 |
| | yl)propylsulphonyl]-4- | | |
| | piperidinyl]-5-fluoro-4-(2- | | |
| | methyl-3-propan-2-yl- | | |
| | imidazol-4-yl)pyrimidin-2- | | |
| | amine | | |
| 158 | N-[1-[3-(Ethyl-methyl- | | 482 |
| | amino)propylsulphonyl]-4- | | |
| | piperidinyl]-5-fluoro-4-(2- | | |
| | methyl-3-propan-2-yl- | | |
| | imidazol-4-yl)pyrimidin-2- | | |
| | amine | | |
| 159 | N-[1-(3-Diethylaminopropylsulphonyl)- | | 496 |
| | 4- | | |
| | piperidinyl]-5-fluoro-4-(2- | | |
| | methyl-3-propan-2-yl- | | |
| | imidazol-4-yl)pyrimidin-2- | | |
| | amine | | |
| 160 | N-[1-[3-(Cyclopropyl- | | 494 |
| | methylamino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |
| 161 | 5-Fluoro-N-[1-[3-(2- | | 512 |
| | methoxyethyl-methyl- | | |
| | amino)propylsulphonyl]-4- | | |
| | piperidinyl]-4-(2-methyl- | | |
| | 3-propan-2-yl-imidazol-4- | | |
| | yl)pyrimidin-2-amine | | |
| 162 | 3-[Ethyl-[3-[[4-[[5-fluoro- | | 521 |
| | 4-(2-methyl-3-propan-2- | | |
| | yl-imidazol-4-yl)pyrimidin- | | |
| | 2-yl]amino]-1- | | |
| | piperidinyl]sulphonyl]propyl]amino]propanenitrile | | |
| 163 | N-[1-[3-(N-Cyclopentyl-N- | | 522 |
| | methyl-amino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |
| 164 | N-[1-[3-(N-Cyclopropyl- | | 494 |
| | N-methyl-amino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |
| 165 | N-[1-[3-(N-Cyclobutyl-N- | | 508 |
| | methyl-amino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |
| 166 | N-[1-[3-(N- | | 508 |
| | Cyclopropylmethyl-N- | | |
| | methyl-amino)propylsulphonyl]- | | |
| | 4-piperidinyl]- | | |
| | 5-fluoro-4-(2-methyl-3- | | |
| | propan-2-yl-imidazol-4- | | |
| | yl)pyrimidin-2-amine | | |

### Example 167

### 4-[[5-Fluoro-4-(2-methyl-3-propan-2-yl-imidazol-4-yl)pyrimidin-2-yl]amino]-N-(2-pyrrolidin-1-ylethyl)piperidine-1-sulphonamide

A solution of 5-fluoro-4-(2-methyl-3-propan-2-yl-imidazol-4-yl)-N-(4-piperidinyl)pyrimidin-2-amine (Example 137) and TEA (0.044 ml, 0.31 mmol) in DCM (3 ml) was cooled to −78° C. and sulfuryl chloride (0.026 ml, 0.31 mmol) added dropwise. The mixture was stirred at −78° C. for 15 mins, then allowed to warm to ambient temperature and TEA (0.044 ml, 0.31 mmol) and 1-(2-aminoethyl)pyrrolidine (44 mg, 0.38 mmol) were added. After stirring for 60 hrs DCM (7 ml) was added and the reaction mixture washed with water (5 ml). The aqueous layer was washed with further DCM (5 ml) and the combined organics passed through a phase separation membrane and evaporated in vacuo. The residue was purified by RPHPLC to give the title compound as a gum (50 mg, 33%). NMR (CDCl₃, 400.132 MHz) 1.52-1.65 (m, 9H), 1.74-1.81 (m, 4H), 2.08-2.17 (m, 2H), 2.47-2.56 (m, 4H), 2.60 (s, 3H), 2.64 (t, 2H), 2.95 (t, 2H), 3.12 (t, 2H), 3.70 (d, 2H), 3.80-3.92 (m, 1H), 4.89 (d, 1H), 5.46-5.59 (m, 1H), 7.53 (d, 1H), 8.15 (d, 1H); MH+ 495.

### Examples 168 to 182

The following compounds were prepared by the procedure of Example 167 and on the same scale by using the appropriate amine.

| Ex | Compound | NMR (400.132 MHz, CDCl₃) | m/z |
|---|---|---|---|
| 168 | N-(2-Diethylaminoethyl)- | 1.02 (t, 6H), 1.50-1.68 (m, 10H), 2.13 (d, | 497 |
| | 4-[[5-fluoro-4-(2-methyl- | 2H), 2.53 (q, 4H), 2.57-2.62 (m, 4H), | |
| | 3-propan-2-yl-imidazol-4- | 2.94 (t, 2H), 3.06 (t, 2H), 3.70 (d, 2H), | |
| | yl)pyrimidin-2- | 3.80-3.91 (m, 1H), 4.90 (d, 1H), 5.46-5.59 (m, 1H), | |
| | yl]amino]piperidine-1- | 7.52 (d, 1H), 8.15 (d, 1H) | |
| | sulphonamide | | |
| 169 | 4-[[5-Fluoro-4-(2-methyl- | | 511 |
| | 3-propan-2-yl-imidazol-4- | | |
| | yl)pyrimidin-2-yl]amino]- | | |
| | N-(2-morpholin-4- | | |
| | ylethyl)piperidine-1- | | |
| | sulphonamide | | |
| 170 | 4-[[5-Fluoro-4-(2-methyl- | | 524 |
| | 3-propan-2-yl-imidazol-4- | | |
| | yl)pyrimidin-2-yl]amino]- | | |
| | N-[2-(4-methylpiperazin- | | |
| | 1-yl)ethyl]piperidine-1- | | |
| | sulphonamide | | |
| 171 | 4-[[5-Fluoro-4-(2-methyl- | | 509 |
| | 3-propan-2-yl-imidazol-4- | | |
| | yl)pyrimidin-2-yl]amino]- | | |
| | N-(3-pyrrolidin-1- | | |
| | ylpropyl)piperidine-1- | | |
| | sulphonamide | | |
| 172 | N-(3-Dimethylamino-2,2- | | 511 |
| | dimethyl-propyl)-4-[[5- | | |
| | fluoro-4-(2-methyl-3- | | |
| | propan-2-yl-imidazol-4- | | |
| | yl)pyrimidin-2-yl]amino]piperidine- | | |
| | 1-sulphonamide | | |
| 173 | 4-[[5-Fluoro-4-(2-methyl- | 1.37-1.64 (m, 14H), 1.71 (t, 2H), 3.11 (d, | 523 |
| | 3-propan-2-yl-imidazol-4- | 2H), 2.31-2.53 (m, 6H), 2.60 (s, 3H), 2.92 (t, | |
| | yl)pyrimidin-2-yl]amino]- | 2H), 3.17 (t, 2H), 3.68 (d, 2H), | |
| | N-[3-(1-piperidinyl)propyl]piperidine- | 3.80-3.91 (m, 1H), 4.88 (d, 1H), 5.48-5.60 (m, 1H), | |
| | 1- | 7.53 (d, 1H), 8.15 (d, 1H) | |
| | sulphonamide | | |
| 174 | 4-[[5-Fluoro-4-(2-methyl- | | 527 |
| | 3-propan-2-yl-imidazol-4- | | |
| | yl)pyrimidin-2-yl]amino]- | | |
| | N-[3-[(3S)-3-fluoropyrrolidin- | | |
| | 1-yl]propyl]piperidine- | | |
| | 1-sulfonamide | | |
| 175 | N-(3-Dimethylamino- | | 497 |
| | propyl)-4-[[5-fluoro-4-(2- | | |
| | methyl-3-propan-2-yl- | | |
| | imidazol-4-yl)pyrimidin-2- | | |
| | yl]amino]-N-methyl- | | |
| | piperidine-1-sulphonamide | | |
| 176 | 4-[[5-Fluoro-4-(2-methyl- | | 509 |
| | 3-propan-2-yl-imidazol-4- | | |
| | yl)pyrimidin-2-yl]amino]- | | |
| | N-[2-(1-methylpyrrolidin- | | |
| | 2-yl)ethyl]piperidine-1- | | |
| | sulphonamide | | |
| 177 | 4-[[5-Fluoro-4-(2-methyl- | | 495 |
| | 3-propan-2-yl-imidazol-4- | | |
| | yl)pyrimidin-2-yl]amino]- | | |
| | N-(1-methyl-4-piperidinyl)piperidine- | | |
| | 1-sulphonamide | | |
| 178 | 4-[[5-Fluoro-4-(2-methyl- | | 523 |
| | 3-propan-2-yl-imidazol-4- | | |
| | yl)pyrimidin-2-yl]amino]- | | |
| | N-(1-propan-2-yl-4- | | |
| | piperidinyl)piperidine-1- | | |
| | sulphonamide | | |
| 179 | N-[1-[(3R)-3- | 1.48-1.61 (m, 7H), 1.77-1.90 (m, 1H), | 495 |
| | Dimethylaminopyrrolidin- | 2.07-2.17 (m, 3H), 2.26 (s, 6H), 2.60 (s, 3H), | |
| | 1-yl]sulfonyl-4- | 2.37-2.84 (m, 1H), 2.97 (t, 2H), 3.09 (t, 1H), | |
| | piperidinyl]-5-fluoro-4-(2- | 3.34 (appq, 2H), 3.46 (appt, 1H), 3.56 (appt, | |
| | methyl-3-propan-2-yl- | 1H), 3.64-3.74 (m, 2H), 3.80-3.92 (m, 1H), | |
| | imidazol-4-yl)pyrimidin-2- | 4.89 (d, 1H), 5.46-5.58 (m, 1H), 7.53 (d, | |
| | amine | 1H), 8.15 (d, 1H) | |
| 180 | 5-Fluoro-N-[1-[(4-methyl- | | 495 |
| | 1,4-diazepan-1- | | |
| | yl)sulphonyl]-4- | | |
| | piperidinyl]-4-(2-methyl- | | |
| | 3-propan-2-yl-imidazol-4- | | |
| | yl)pyrimidin-2-amine | | |
| 181 | 5-Fluoro-N-[1-(4- | 1.54-1.63 (m, 7H), 2.10 (m, 3H), 2.32 (s, | 481 |
| | methylpiperazin-1- | 3H), 2.46 (t, 4H), 2.60 (s, 3H), 2.98 (t, 2H), | |
| | yl)sulphonyl-4- | 3.28 (t, 4H), 3.71 (d, 2H), 3.80-3.92 (m, | |
| | piperidinyl]-4-(2-methyl- | 1H), 4.87 (d, 1H), 4.46-5.57 (m, 1H), | |
| | 3-propan-2-yl-imidazol-4- | 7.53 (d, 1H), 8.15 (d, 1H) | |
| | yl)pyrimidin-2-amine | | |
| 182 | N-(2-Dimethylamino- | | 469 |
| | ethyl)-4-[[5-fluoro-4-(2- | | |
| | methyl-3-propan-2-yl- | | |
| | imidazol-4-yl)pyrimidin-2- | | |
| | yl]amino]piperidine-1- | | |
| | sulphonamide | | |

### Example 183

### Benzyl 3-[[4-[[5-fluoro-4-(2-methyl-3-propan-2-yl-imidazol-4-yl)pyrimidin-2-yl]amino]-1-piperidinyl]sulfonylmethyl]piperidine-1-carboxylate

5-Fluoro-4-(2-methyl-3-propan-2-yl-imidazol-4-yl)-N-(4-piperidinyl)pyrimidin-2-amine (Example 137; 0.5 g, 1.57 mmol) and TEA (0.33 ml, 2.35 mmol) were dissolved in DCM (50 ml), then benzyl 3-(chlorosulfonylmethyl)piperidine-1-carboxylate (Preparation 62in WO99/45006; 0.82 g, 2.35 mmol) was added. The reaction was stirred for 1 hr, then evaporated to dryness and passed through a SCX column. The material obtained was then purified by flash chromatography on silica, eluting with 0-5% MeOH in DCM, to give the title compound as a yellow foam (0.4 g, 41%). MH+ 614.

### Example 184

### Benzyl 4-[[4-[[5-fluoro-4-(2-methyl-3-propan-2-yl-imidazol-4-yl)pyrimidin-2-yl]amino]-1-piperidinyl]sulphonyl]piperidine-1-carboxylate

Benzyl 4-chlorosulfonylpiperidine-1-carboxylate (334 mg) in DCM (3 ml) was added dropwise to a stirred solution of 5-fluoro-4-(2-methyl-3-propan-2-yl-imidazol-4-yl)-N-(4-piperidinyl)pyrimidin-2-amine (Example 137; 318 mg) and TEA (202 mg) in dry DCM (9 ml) at ambient temperature. After 2 hrs the mixture was diluted with sat. aq. NaHCO₃ and extracted with DCM. The extract was dried, concentrated and purified by flash chromatography on silica eluting with a gradient of 0-10% MeOH in DCM to give the title compound as a solid foam (570 mg, 95%). NMR (400.13 MHz, CDCl₃) 1.50-1.80 (m, 10H), 2.10 (m, 4H), 2.60 (s, 3H), 2.80 (m, 2H), 3.05 (m, 3H), 3.79 (m, 2H), 3.90 (m, 1H), 4.35 (m, 2H), 4.90 (d, 1H), 5.13 (s, 2H), 5.50 (m, 1H), 7.37 (m, 5H), 7.52 (d, 1H), 8.15 (d, 1H); MH+

### Example 185

### 5-Fluoro-4-(2-methyl-3-propan-2-yl-imidazol-4-yl)-N-[1-(4-piperidinylsulphonyl)-4-piperidinyl]pyrimidin-2-amine

Benzyl 4-[[4-[[5-fluoro-4-(2-methyl-3-propan-2-yl-imidazol-4-yl)pyrimidin-2-yl]amino]-1-piperidinyl]sulphonyl]piperidine-1-carboxylate (Example 184; 480 mg) in MeOH (16 ml) was hydrogenated using a H-CUBE system (Thales Nanotechnology) with 10% Pd/C cartridge at 50° C., using full flow hydrogen (1 bar, 1 ml/min). The solvent was evaporated to give the product as a gum (344 mg, 92%). NMR (400.13 MHz, CDCl₃) 1.50-1.75 (m, 10H), 2.08 (m, 4H), 2.60 (m, 5H), 3.08 (m, 3H), 3.22 (m, 2H), 3.82 (m, 2H), 3.91 (m, 1H), 4.92 (d, 1H), 5.50 (m, 1H), 7.52 (d, 1H), 8.15 (d, 1H); MH+ 466.

### Example 186

### 5-Fluoro-N-[1-[(1-methyl-4-piperidinyl)sulfonyl]-4-piperidinyl]-4-(2-methyl-3-propan-2-yl-imidazol-4-yl)pyrimidin-2-amine

5-Fluoro-4-(2-methyl-3-propan-2-yl-imidazol-4-yl)-N-[1-(4-piperidinylsulphonyl)-4-piperidinyl]pyrimidin-2-amine (Example 185; 70 mg) was stirred with formaldehyde (37% aqueous solution, 60 mg) in MeOH (1 ml) at ambient temperature. Sodium cyanoborohydride (1 M solution in THF, 0.3 ml) was added and stirred for 1 hr then the solvent was evaporated. The residue was diluted with 2M NaOH and extracted with DCM. The combined extracts were washed with brine and concentrated in vacuo. Purification by RPHPLC gave the title compound as a colourless oil (54 mg, 75%). NMR (400.13 MHz, CDCl₃) 1.55 (m, 8H), 1.80-2.00 (m, 4H), 2.10 (m, 4H), 2.28 (s, 3H), 2.60 (s, 3H), 2.90 (m, 1H), 2.97 (m, 2H), 3.08 (m, 2H), 3.81 (m, 2H), 3.90 (m, 1H), 4.88 (d, 1H), 5.50 (m, 1H), 7.52 (d, 1H), 8.15 (d, 1H); MH+ 480.

### Example 187

### 5-Fluoro-4-(2-methyl-3-propan-2-yl-imidazol-4-yl)-N-[1-[(1-propan-2-yl-4-piperidinyl)sulfonyl]-4-piperidinyl]pyrimidin-2-amine

5-Fluoro-4-(2-methyl-3-propan-2-yl-imidazol-4-yl)-N-[1-(4-piperidinylsulphonyl)-4-piperidinyl]pyrimidin-2-amine (Example 185; 70 mg) was stirred with acetone (35 mg) in MeOH (1 ml) at ambient temperature. Sodium cyanoborohydride (1 M solution in THF, 0.3 ml) was added and stirred for 16 hrs. Concentrated HCl (diluted in MeOH) was added dropwise to adjust the pH to 6, then additional acetone (200 mg) and sodium cyanoborohydride (1 M solution in THF, 0.3 ml) was added and the mixture stirred for 3 hrs. After which the residue was diluted with 2M NaOH and extracted with DCM. The combined extracts were washed with brine and concentrated in vacuo then purified by RPHPLC to give the title compound as a colourless oil (54 mg, 71%). NMR (400.13 MHz, CDCl₃) 1.03 (d, 6H), 11.55 (m, 8H), 1.80 (m, 2H), 2.10 (m, 6H), 2.60 (s, 3H), 2.74 (m, 1H), 2.87 (m, 1H), 2.98 (m, 2H), 3.07 (m, 1H), 3.80 (m, 2H), 3.90 (m, 1H), 4.90 (m, 1H), 5.50 (s, 1H), 7.52 (d, 1H), 8.15 (d, 1H); MH+ 508.

### Example 188

### Benzyl 6-[[5-fluoro-4-(2-methyl-3-propan-2-yl-imidazol-4-yl)pyrimidin-2-yl]amino]-(1α, 5α, 6α)-3-azabicyclo[3.1.0]hexane-3-carboxylate

2-Chloro-5-fluoro-4-(2-methyl-3-propan-2-yl-imidazol-4-yl)pyrimidine (Method 8; 1.53 g) was stirred and heated with benzyl (1α, 5α, 6α)-6-amino-3-azabicyclo[3.1.0]hexane-3-carboxylate (Preparation 2 in WO97/19942; 1.54 g) and DIPEA (0.93 g) in DMA (12 ml) at > 125° C. for 10 hrs. The mixture was concentrated by evaporation then diluted with 2M aq. sodium carbonate and extracted with DCM. The extracts were purified by flash chromatography on silica, eluting with a gradient of 0-100% EtOAc in DCM to give the title compound as a gum (800 mg, 22%). NMR 1.42 (d, 6H), 1.77 (m, 2H), 2.49 (s, 3H), 3.46 (m, 2H), 3.61 (m, 2H), 5.06 (m, 2H), 5.50 (m, 1H), 7.40 (m, 7H), 8.36 (d, 1H); MH+ 451.

### Example 189

### N-[5-Fluoro-4-(2-methyl-3-propan-2-yl-imidazol-4-yl)pyrimidin-2-yl]-(1α, 5α, 6α)-3-azabicyclo[3.1.0]hexan-6-amine

Benzyl 6-[[5-fluoro-4-(2-methyl-3-propan-2-yl-imidazol-4-yl)pyrimidin-2-yl]amino]-> (1α, 5α, 6α)-3-azabicyclo[3.1.0]hexane-3-carboxylate (Example 188; 720 mg) in EtOH (36 ml) was hydrogenated in the presence of 10% Pd/C (300 mg) at 1 atmosphere pressure and 40° C. for 2 hrs. The catalyst was filtered through diatomaceous earth and the filtrate was evaporated to give the title compound as a gum (500 mg, 98%). NMR (400.13 MHz, CDCl₃+D₂O) 1.60 (m, 8H), 2.60 (m, 4H), 2.95 (d, 2H), 3.15 (d, 2H), 5.56 (m, 1H), 7.52 (d, 1H), 8.16 (d, 1H); MH+ 317.

### Example 190

### N-[5-Fluoro-4-(2-methyl-3-propan-2-yl-imidazol-4-yl)pyrimidin-2-yl]-3-methylsulphonyl-(1α, 5α, 6α)-3-azabicyclo[3.1.0]hexan-6-amine

N-[5-Fluoro-4-(2-methyl-3-propan-2-yl-imidazol-4-yl)pyrimidin-2-yl]-(1α, 5α, 6α)-3-azabicyclo[3.1.0]hexan-6-amine (Example 189; 65 mg, 0.21 mmol) was dissolved in DCM (2 ml). Mesyl chloride (0.025 ml, 0.32 mmol) was added then TEA (0.06 ml, 0.42 mmol) and the mixture stirred at ambient temperature for 16 hrs. The solvent was evaporated and the residue purified by flash chromatography on silica, eluting with 5% MeOH/DCM, to give the title compound as a gum (33 mg, 40%). NMR (400.132 MHz) 1.56 (d, 6H), 1.87 (s, 2H), 2.56 (s, 3H), 2.81 (s, 1H), 2.96 (s, 3H), 3.46 (m, 4H), 5.60 (m, 1H), 7.40 (s, 1H), 7.45 (s, 1H), 8.43 (s, 1H); MH+ 395.

### Examples 191 to 193

The following examples were prepared in a similar manner to Example 139 and on a similar scale by using N-[5-fluoro-4-(2-methyl-3-propan-2-yl-imidazol-4-yl)pyrimidin-2-yl]-(1α, 5α, 6α)-3-azabicyclo[3.1.0]hexan-6-amine (Example 189) and the appropriate amine.

| Ex | Compound | NMR (400.132 MHz, CDCl₃) | m/z |
|---|---|---|---|
| 191 | N-[5-Fluoro-4-(2-methyl-3- | 1.56 (d, 6H), 1.78 (m, 6H), 2.53 (m, | 478 |
| | propan-2-yl-imidazol-4- | 4H), 2.59 (s, 3H), 2.82 (d, 1H), | |
| | yl)pyrimidin-2-yl]-3-(2- | 2.91 (t, 3H), 3.18 (t, 2H), 3.48 (m, 2H), | |
| | pyrrolidin-1-ylethylsulphonyl)- | 3.73 (d, 2H), 5.07 (d, 1H), 5.55 (m, | |
| | (1α,5α,6α)-3- | 1H), 7.54 (d, 1H), 8.17 (d, 1H) | |
| | azabicyclo[3.1.0]hexan-6-amine | | |
| 192 | 3-(2- | 1.56 (d, 6H), 1.77 (s, 2H), 2.26 (s, | 452 |
| | Dimethylaminoethylsulphonyl)- | 6H), 2.59 (s, 3H), 2.76 (m, 2H), | |
| | N-[5-fluoro-4-(2-methyl-3- | 2.83 (d, 1H), 3.13 (m, 2H), 3.46 (m, 2H), | |
| | propan-2-yl-imidazol-4- | 3.73 (d, 2H), 5.09 (d, 1H), 5.55 (m, | |
| | yl)pyrimidin-2-yl]-(1α,5α,6α)-3- | 1H), 7.54 (d, 1H), 8.17 (d, 1H) | |
| | azabicyclo[3.1.0]hexan-6-amine | | |
| 193 | 3-[2-(7-Azabicyclo[2.2.1]hept-7- | 1.32 (m, 4H), 1.56 (d, 6H), 1.72 (m, | 504 |
| | yl)ethylsulphonyl]-N-[5-fluoro-4- | 6H), 2.59 (s, 3H), 2.79 (m, 2H), | |
| | (2-methyl-3-propan-2-yl- | 2.84 (d, 1H), 3.14 (m, 2H), 3.25 (m, 2H), | |
| | imidazol-4-yl)pyrimidin-2-yl]- | 3.49 (m, 2H), 3.72 (d, 2H), 5.08 (d, | |
| | (1α,5α,6α)-3- | 1H), 5.55 (m, 1H), 7.54 (d, 2H), | |
| | azabicyclo[3.1.0]hexan-6-amine | 8.17 (d, 2H) | |

### Example 194

### N-[5-Fluoro-4-(2-methyl-3-propan-2-yl-imidazol-4-yl)pyrimidin-2-yl]-3-(3-pyrrolidin-1-ylpropylsulphonyl)-(1α, 5α, 6α)-3-azabicyclo[3.1.0]hexan-6-amine

N-[5-Fluoro-4-(2-methyl-3-propan-2-yl-imidazol-4-yl)pyrimidin-2-yl]-(1α, 5α, 6α)-3-azabicyclo[3.1.0]hexan-6-amine (Example 189; 285 mg) and DIPEA (349 mg) were dissolved in DCM (15 ml) and cooled to −10° C. 3-Chloropropane sulfonyl chloride (176 mg) was then added dropwise in DCM (2 ml). The reaction mixture was warmed to ambient temperature and stirred for 30 mins, then evaporated to dryness. The residue was dissolved in DMF (6 ml) and separated into 6×1 ml aliquots. DIPEA (39 mg) and pyrrolidine (300 mg) was added to an aliquot and the reaction mixture heated at 70° C. for 16 hrs. The reaction mixture was concentrated then purified by RPHPLC to give the title compound as a gum (56 mg, 73%). NMR (400.13 MHz, CDCl₃) 1.57 (d, 6H), 1.80 (m, 6H), 2.00 (m, 2H), 2.55 (m, 9H), 2.83 (d, 1H), 3.05 (m, 2H), 3.46 (m, 2H), 3.72 (d, 2H), 5.08 (d, 1H), 5.57 (m, 1H), 7.54 (d, 1H), 8.17 (d, 1H); MH+ 492.

### Examples 195 to 199

The following compounds were prepared by the procedure of Example 194 and on the same scale by using the remaining aliquots with the appropriate amine.

| Ex | Compound | NMR (400.132 MHz, CDCl₃) | m/z |
|---|---|---|---|
| 195 | N-[5-Fluoro-4-(2-methyl-3- | 1.43 (m, 2H), 1.55 (m, 10H), | 506 |
| | propan-2-yl-imidazol-4- | 1.78 (m, 2H), 1.97 (m, 2H), 2.36 (m, 6H), | |
| | yl)pyrimidin-2-yl]-3-[3-(1- | 2.59 (s, 3H), 2.83 (d, 1H), 3.02 (m, | |
| | piperidinyl)propylsulphonyl]-(1α, | 2H), 3.47 (m, 2H), 3.72 (d, 2H), | |
| | 5α,6α)-3-azabicyclo[3.1.0]hexan- | 5.09 (d, 1H), 5.57 (m, 1H), 7.54 (d, 1H), | |
| | 6-amine | 8.17 (d, 1H) | |
| 196 | 3-[3-(N-Cyclopentyl-N-methyl- | | 520 |
| | amino)propylsulphonyl]-N-[5- | | |
| | fluoro-4-(2-methyl-3-propan-2-yl- | | |
| | imidazol-4-yl)pyrimidin-2-yl]- | | |
| | (1α,5α,6α)-3- | | |
| | azabicyclo[3.1.0]hexan-6-amine# | | |
| 197 | 3-[3-(2,5-Dimethylpyrrolidin-1- | | 520 |
| | yl)propylsulphonyl]-N-[5-fluoro- | | |
| | 4-(2-methyl-3-propan-2-yl- | | |
| | imidazol-4-yl)pyrimidin-2-yl]- | | |
| | (1α,5α,6α)-3- | | |
| | azabicyclo[3.1.0]hexan-6-amine# | | |
| 198 | 3-(3- | 1.58 (d, 6H), 1.77 (s, 2H), 1.96 (m, | 466 |
| | Dimethylaminopropylsulphonyl)- | 2H), 2.21 (s, 6H), 2.37 (t, 2H), | |
| | N-[5-fluoro-4-(2-methyl-3- | 2.59 (s, 3H), 2.83 (d, 1H), 3.02 (m, 2H), | |
| | propan-2-yl-imidazol-4- | 3.48 (m, 2H), 3.73 (m, 2H), 5.08 (d, | |
| | yl)pyrimidin-2-yl]-(1α,5α,6α)-3- | 1H), 5.56 (m, 1H), 7.54 (d, 1H), | |
| | azabicyclo[3.1.0]hexan-6-amine* | 8.17 (d, 1H) | |
| 199 | 3-[3-(7-Azabicyclo[2.2.1]hept-7- | 1.29 (m, 4H), 1.60 (d, 6H), 1.71 (m, | 518 |
| | yl)propylsulphonyl]-N-[5-fluoro- | 4H), 1.79 (s, 2H), 1.97 (m, 2H), | |
| | 4-(2-methyl-3-propan-2-yl- | 2.47 (t, 2H), 2.59 (s, 3H), 2.83 (d, 1H), | |
| | imidazol-4-yl)pyrimidin-2-yl]- | 3.11 (m, 2H), 3.26 (s, 2H), 3.46 (m, | |
| | (1α,5α,6α)-3- | 2H), 3.73 (d, 2H), 5.08 (d, 1H), | |
| | azabicyclo[3.1.0]hexan-6-amine# | 5.56 (m, 1H), 7.54 (d, 1H), 8.17 (d, 1H) | |

*Performed at ambient temperature for 7 days
#Performed at 95° C. for 2 days

### Example 200

Benzyl 4-[[4-(3-cyclopentyl-2-methyl-imidazol-4-yl)-5-fluoro-pyrimidin-2-yl]amino]piperidine-1-carboxylate

(E)-1-(3-Cyclopentyl-2-methyl-imidazol-4-yl)-3-dimethylamino-prop-2-en-1-one (Method 13; 6.7 g, 27.1 mmol) was added to dry acetonitrile (140 ml) and cooled to −2° C. then Selectfluor™ (12 g, 33.9 mmol) was added portionwise, maintaining the temperature at −2° C. The reaction was then allowed to warm to ambient temperature and stirred for 30 mins before being evaporating to dryness. Saturated aq. NaHCO₃ was added then the aqueous layer was extracted with DCM (3×200 ml), dried and the solvent removed in vacuo to yield a yellow gum. The gum and benzyl 4-carbamimidamidopiperidine-1-carboxylate (Method 14; 8.2 g, 29.8 mmol) were added to 2-methoxyethanol (100 ml) and heated under reflux for 16 hrs. The reaction was then evaporated to dryness, sat. aq. NaHCO₃ was added, then extracted with DCM (3×150 ml), dried and the solvent removed in vacuo to yield a viscous black oil. Purification by flash chromatography on silica, eluting with 0-5% MeOH in DCM gave a solid which was dissolved in DCM, acetonitrile was added then the DCM was slowly removed in vacuo to precipitate a solid. The precipitate was filtered and dried in vacuo to give the title compound as a colourless solid. NMR (400.132 MHz, CDCl₃) 1.40-1.48 (m, 2H), 1.62-1.73 (m, 2H), 1.88-2.05 (m, 6H), 2.11-2.19 (m, 2H), 2.57 (s, 3H), 2.97-3.03 (m, 2H), 3.85-3.94 (m, 1H), 4.14 (d, 2H), 4.90 (d, 1H), 5.14 (s, 2H), 5.57-5.62 (m, 1H), 7.29-7.37 (m, 5H), 7.51 (d, 11H), 8.15 (d, 1H); MH+ 479.

### Example 201

### 4-(3-Cyclopentyl-2-methyl-imidazol-4-yl)-5-fluoro-N-(4-piperidinyl)pyrimidin-2-amine

Benzyl 4-[[4-(3-cyclopentyl-2-methyl-imidazol-4-yl)-5-fluoro-pyrimidin-2-yl]amino]piperidine-1-carboxylate (Example 200; 4.5 g, 11.3 mmol) and 10% Pd/C (0.45 g) in EtOH (100 ml) were stirred at 40° C. under hydrogen at 5 bar pressure for 16 hrs. The reaction mixture was filtered through diatomaceous earth and the solvent removed to yield a waxy solid. This was passed through a short column of silica eluting with 3.5-10% MeOH in DCM. The gum obtained was recrystallised from hot acetonitrile to give the title compound. NMR (400.132 MHz, CDCl₃) 1.41 (ddd, 2H), 1.65-1.72 (m, 2H), 1.89-2.05 (m, 6H), 2.11-2.21 (m, 2H), 2.58 (s, 3H), 2.67-2.74 (m, 2H), 3.11-3.14 (m, 2H), 3.77-3.86 (m, 1H), 4.94 (d, 1H), 5.61-5.73 (m, 1H), 7.51 (d, 1H), 8.14 (d, 1H); MH+ 345.

### Example 202

### 4-(3-Cyclopentyl-2-methyl-imidazol-4-yl)-5-fluoro-N-(1-methylsulphonyl-4-piperidinyl)pyrimidin-2-amine

The title compound was prepared in a similar manner to Example 115 and on a similar scale by using 4-(3-cyclopentyl-2-methyl-imidazol-4-yl)-5-fluoro-N-(4-piperidinyl)pyrimidin-2-amine (Example 201) as the starting material. NMR (400.132 MHz, CDCl₃) 1.61-1.73 (m, 4H), 1.89-2.08 (m, 4H), 2.13-2.17 (m, 4H), 2.58 (s, 3H), 2.81 (s, 3H), 2.91 (dt, 2H), 3.74-3.77 (m, 2H), 3.83-3.92 (m, 1H), 4.98 (d, 1H), 5.53 (quintet, 1H), 7.51 (d, 1H), 8.16 (d, 1H); MH+ 423.

### Examples 203 to 204

The following examples were prepared in a similar manner to Example 139 and on a similar scale by using 4-(3-cyclopentyl-2-methyl-imidazol-4-yl)-5-fluoro-N-(4-piperidinyl)pyrimidin-2-amine (Example 201) as the starting material.

| Ex | Compound | NMR (400.132 MHz, CDCl₃) | m/z |
|---|---|---|---|
| 203 | 4-(3-Cyclopentyl-2-methyl- | 1.57-1.72 (m, 4H), 1.79-1.83 (m, 4H), | 506 |
| | imidazol-4-yl)-5-fluoro-N-[1- | 1.85-2.19 (m, 8H), 2.54-2.58 (m, 7H), | |
| | (2-pyrrolidin-1- | 2.90-3.02 (m, 4H), 3.14-3.18 (m, 2H), 3.76-3.79 (m, | |
| | ylethylsulphonyl)-4- | 2H), 3.83-3.92 (m, 1H), 4.93 (d, 1H), | |
| | piperidinyl]pyrimidin-2-amine | 5.53 (quintet, 1H), 7.50 (d, 1H), 8.16 (d, 1H) | |
| 204 | 4-(3-Cyclopentyl-2-methyl- | 1.57-1.70 (m, 4H), 1.88-2.19 (m, 8H), | 480 |
| | imidazol-4-yl)-N-[1-(2- | 2.28 (s, 6H), 2.58 (s, 3H), 2.76 (t, 2H), | |
| | dimethylaminoethylsulphonyl)- | 2.96-3.02 (m, 2H), 3.10 (t, 2H), 3.76-3.79 (m, 2H), | |
| | 4-piperidinyl]-5-fluoro- | 3.85-3.94 (m, 1H), 4.96 (d, 1H), | |
| | pyrimidin-2-amine | 5.54 (quintet, 1H), 7.50 (d, 1H), 8.16 (d, 1H) | |

### Example 205

### N-[1-(3-Chloropropylsulfonyl)-4-piperidinyl]-4-(3-cyclopentyl-2-methyl-imidazol-4-yl)-5-fluoro-pyrimidin-2-amine

The title compound was prepared in a similar manner to Example 143 and on a similar scale by using 4-(3-cyclopentyl-2-methyl-imidazol-4-yl)-5-fluoro-N-(4-piperidinyl)pyrimidin-2-amine (Example 201) as the starting material. MH+ 487.

### Examples 206 to 207

The following compounds were prepared by the procedure of Example 144 and on the same scale by using N-[1-(3-chloropropylsulfonyl)-4-piperidinyl]-4-(3-cyclopentyl-2-methyl-imidazol-4-yl)-5-fluoro-pyrimidin-2-amine (Example 205) and the appropriate amine.

| Ex | Compound | NMR (400.132 MHz, CDCl₃) | m/z |
|---|---|---|---|
| 206 | 4-(3-Cyclopentyl-2-methyl- | 1.57-1.72 (m, 4H), 1.92-2.17 (m, 10H), | 494 |
| | imidazol-4-yl)-N-[1-(3- | 2.22 (s, 6H), 2.39 (t, 2H), 2.58 (s, 3H), | |
| | dimethylaminopropylsulphonyl)- | 2.95-3.02 (m, 4H), 3.77 (d, 2H), | |
| | 4-piperidinyl]-5- | 3.83-3.92 (m, 1H), 5.01 (d, 1H), 5.54 (quintet, 1H), | |
| | fluoro-pyrimidin-2-amine | 7.50 (d, 1H), 8.16 (d, 1H) | |
| 207 | 4-(3-Cyclopentyl-2-methyl- | 1.62-1.74 (m, 4H), 1.89-2.19 (m, 12H), | 520 |
| | imidazol-4-yl)-5-fluoro-N-[1-(3- | 2.30 (quintet, 2H), 2.57 (s, 3H), | |
| | pyrrolidin-1-ylpropylsulphonyl)- | 3.01-3.09 (m, 8H), 3.20 (t, 2H), 3.76-3.80 (m, 2H), | |
| | 4-piperidinyl]pyrimidin-2-amine | 3.86-3.95 (m, 1H), 5.21 (d, 1H), | |
| | | 5.54 (quintet, 1H), 7.49 (d, 1H), 8.15 (d, 1H) | |

### Example 208

### tert-Butyl 3-[[5-fluoro-4-(2-methyl-3-propan-2-yl-imidazol-4-yl)pyrimidin-2-yl]amino]azepane-1-carboxylate

A solution of tert-butyl 3-carbamimidamidoazepane-1-carboxylate (Method 15; 192mg, 0.75 mmol) and (2Z)-3-(dimethylamino)-2-fluoro-1-(1-isopropyl-2-methyl-1H-imidazol-5-yl)prop-2-en-1-one (Method 1 in WO07015064) (360 mg, 1.50 mmol) in 2-methoxyethanol (10 ml) were heated at reflux for 24 hrs. The solvent was then evaporated and the residue purified by flash chromatography on silica, eluting with 0-5% MeOH/DCM to give the title compound as a colourless solid (48 mg, 15%). NMR (400.132 MHz, CDCl₃) 1.43 (m, 16H), 1.65 (m, 4H), 1.92 (m, 1H), 2.52 (s, 3H), 3.06 (m, 1H), 3.39 (m, 1H), 3.63 (m, 2H), 4.08 (m, 1H), 5.50 (m, 1H), 7.19 (s, 1H), 7.44 (d, 1H), 8.06 (s, 1H); m/z 433.

### Example 209

### N-[5-Fluoro-4-(2-methyl-3-propan-2-yl-imidazol-4-yl)pyrimidin-2-yl]azepan-3-amine

Trifluoroacetic acid (0.5 ml) was added to a stirred solution of tert-butyl 3-[[5-fluoro-4-(2-methyl-3-propan-2-yl-imidazol-4-yl)pyrimidin-2-yl]amino]azepane-1-carboxylate (Example 208; 48 mg, 0.11 mol) in DCM (1.0 ml) at ambient temperature. After 2.5 hours the solvent was evaporated to give the title compound as a gum (103 mg). M/z 333.

### Example 210

### N-[5-Fluoro-4-(2-methyl-1-propan-2-yl-imidazol-4-yl)pyrimidin-2-yl]-1-methylsulfonyl-azepan-3-amine

Methanesulphonyl chloride (0.013 ml, 0.17 mmol) and triethylamine (0.046 ml, 0.33 mmol) were added to a stirred solution of N-[5-fluoro-4-(2-methyl-3-propan-2-yl-imidazol-4-yl)pyrimidin-2-yl]azepan-3-amine (Example 209; 103 mg) in DCM (2 ml) at ambient temperature. After 4 hrs additional methanesulphonyl chloride (0.013 ml, 0.17 mmol) was added and the reaction mixture was stirred at ambient temperature for 64 hrs. The solvent was evaporated to give a gum, which was dissolved in MeOH and loaded onto an SCX-2 column. The column was washed three times with MeOH then eluted with 2M NH₃/MeOH. Additional purification using RPHPLC gave the title compound as a colourless solid (7 mg, 16%). NMR (400.132 MHz, MeOH) 1.65 (d, 6H), 1.82 (m, 6H), 2.03 (m, 2H), 2.81 (s, 3H), 2.87 (s, 3H), 3.38 (m, 4H), 4.16 (m, 1H), 5.53 (m, 1H), 7.84 (s, 1H), 8.40 (s, 1H); m/z 411.

### Preparation of Starting Materials

### Method 1

### Benzyl 4-carbamimidamidopiperidine-1-carboxylate

Benzyl 4-aminopiperidine-1-carboxylate (25 g, 106.7 mmol), pyrazole-1-carboximidamide (31.3 g, 213.4 mmol) and TEA (30 ml) were dissolved in MeCN (500 ml) and heated at 60° C. overnight. After 18 hrs, solvents were evaporated. The resultant orange residue was partitioned between saturated aq NaHCO₃ (500 ml) and DCM (500 ml). The DCM layer was separated and the fine precipitate contained within it was collected by filtration then washed with a little DCM and dried under vacuum to yield a white solid. (30.8 g, 100%). NMR (400.132 MHz) 1.31 (m, 2H), 1.82 (m, 2H), 2.96 (m, 2H), 3.57 (m, 1H), 3.92 (m, 2H), 5.08 (s, 2H), 7.35 (m, 5H), 7.96 (s, 2H).

### Method 2

### tert-Butyl 3-carbamimidamidopyrrolidine-1-carboxylate

To a solution of tert-butyl 3-aminopyrrolidine-1-carboxylate (3.03 g, 16.27 mmol) in acetonitrile (60 ml) was added TEA (4.7 ml, 32.75 mmol) followed by 1H-pyrazole-1-carboxamidine hydrochloride (4.8 g, 33.72 mmol). The reaction mixture was heated to 65° C. (internal temperature) for 6 hrs and then left to cool overnight. The reaction mixture evaporated to yield a salmon pink coloured viscous oil, which was partitioned between sat. aq. NaHCO₃ solution (75 ml) and DCM (75 ml). The mixture was then shaken vigorously and allowed to stand for 10 mins before shaking again and collecting the resultant solid by filtration. The filter cake was washed with DCM, water and pulled dry under suction for ˜30mins before transferring to a vacuum dessicator and left to dry over the weekend, to afford the title compound as an off white solid (2.14 g, 58%). NMR (400.132 MHz) 1.41 (s, 9H), 1.78 (m, 1H), 2.09 (m, 1H), 3.07 (m, 1H), 3.15-3.51 (m, 4H), 4.03 (m, 1H), 7.01-8.81 (m, 3H).

### Method 3

### 4-Morpholin-4-yl butanoic acid hydrochloride

Ethyl 4-bromobutanoate (67 ml, 0.5 M) was added drop-wise to a solution of morpholine (175 ml, 2 M) in dry toluene (11). The reaction mixture was stirred for 4 hrs at 60° C. and then for 16 hrs at ambient temperature. The reaction mixture was filtered at 0° C. and the filtrate evaporated. The resultant material was triturated with 60-80 petrol and evaporated to give an orange oil (91.4 g), which was distilled at reduced pressure to give a clear oil (73.2 g) b.p. 90.2° C./3-4 mm Hg. The resultant oil was heated at reflux for 16 hrs in 18% HCl (aq) (11). The acid was evaporated leaving a sticky solid which on trituration with ether gave a white solid (75.25 g) which was recrystallized from glacial acetic acid/acetone to give the title compound as a white crystalline solid (56.43 g, 53%>) m.p. 181-3° C.

### Method 4

### 4-(2-Methyl-3-propan-2-yl-imidazol-4-yl)pyrimidin-2-ol

4-(2-Methyl-3-propan-2-yl-imidazol-4-yl)pyrimidin-2-amine (Method 39) WO2003/076436, 5 g, 23 mmol) was dissolved in 70% AcOH—water (145 ml) under an inert atmosphere. Sodium nitrite (5.52 g, 80 mmol) in water (10 ml) was added drop-wise at ambient temperature over a 5 minute period giving a mild exotherm. The reaction mixture was heated slowly to 60° C., and held at this temperature for 3 hrs. The reaction mixture was cooled to ambient temperature and neutralised to pH 7 with 40% aq NaOH, extracted with EtOAc (250 ml×5) and the combined extracts dried and evaporated to give the title compound as an off-white solid. (8.2 g 43%). NMR (400.132 MHz, CDCl₃) 1.51 (d, 6H), 2.03 (s, 3H), 2.54 (s, 3H), 5.93 (m, 1H), 6.60 (d, 1H), 7.57 (m, 2H); MH+ 219.

### Method 5

### 2-Chloro-4-(2-methyl-3-propan-2-yl-imidazol-4-yl)pyrimidine

4-(2-Methyl-3-propan-2-yl-imidazol-4-yl)pyrimidin-2-ol (Method 4; 8.2 g, 29.4 mmol), phosphorous oxychloride (120 ml) and phosphorous pentachloride (6.6 g) were combined at heated at reflux for 18 hrs. Excess phosphorus oxychloride was evaporated off and the residue dissolved in DCM and stirred in ice and water. The mixture was taken to pH11 by the addition of 40% aqueous Sodium Hydroxide. The organic and aqueous phases were separated and the organic phased washed with brine, dried and evaporated. The resultant material was dissolved in DCM and chromatographed on silica eluting on a shallow gradient of 0-5% MeOH/DCM. Fractions containing product were combined and evaporated to give the title compound as a pale brown gum. (5.8 g, 84%). NMR (400.132 MHz) 1.53 (d, 6H), 2.50 (s, 3H), 5.27 (m, 1H), 7.72 (s, 1H), 7.79 (d, 1H), 8.62 (d, 1H); MH+ 237.

### Method 6

### 2,5-Dichloro-4-(2-methyl-3-propan-2-yl-imidazol-4-yl)pyrimidine

5-Chloro-4-(2-methyl-3-propan-2-yl-imidazol-4-yl)pyrimidin-2-amine (Method 5 in WO05/075461; 5 g, 19.9 mmol) was dissolved in acetic acid (70 ml)/water (30 ml) and sodium nitrite (2.75 g, 39.8 mmol) in water (8 ml) was slowly added. The reaction was stirred for 10 mins at ambient temperature before heating at 60° C. for 3 hrs. The reaction was then evaporated to dryness, aqueous NaHCO₃ added to pH 9 and the aqueous layer extracted with DCM (3×100 ml). The combined organics were dried, filtered and the solvent removed in vacuo to give a solid which was dissolved in a minimum amount of hot acetonitrile, on cooling a white solid was obtained. The solid was added to phosphorous oxychloride (50 ml) and heated at 80° C. for 1 hr. The reaction mixture was evaporated to dryness, then cautiously quenched with sat. aq. NaHCO₃ to pH 8. The reaction was concentrated to dryness to give the title compound as a yellow solid (3.47 g, 64%). NMR (400.132 MHz, CDCl₃) 1.58 (d, 6H), 2.61 (s, 3H), 4.96 (septet, 1H), 7.80 (s, 1H), 8.58 (s, 1H); MH+ 273.

### Method 7

### 5-Fluoro-4-(2-methyl-3-propan-2-yl-imidazol-4-yl)pyrimidin-2-ol acetate

5-Fluoro-4-(2-methyl-3-propan-2-yl-imidazol-4-yl)pyrimidin-2-amine (Method 17 in WO2006/064251; 4 g, 17 mmol) was dissolved in 70% AcOH—water (108 ml) under an inert atmosphere. Sodium nitrite (4.08 g, 59.2 mmol) in water (8 ml) was added dropwise at ambient temperature over 5 mins. The reaction mixture was warmed slowly to 60° C. After 3 hrs the reaction mixture was cooled then neutralised to pH 7 with 40% aq NaOH. The aqueous layer was extracted with EtOAc (300 ml×6) the combined organics dried, filtered and evaporated to give the title compound as a yellow solid (4.07 g, 81%). NMR (400.132 MHz) 1.48 (d, 6H), 1.91 (s, 3H), 2.50 (s, 3H), 5.44 (m, 1H), 7.47 (d, 1H), 8.29 (d, 1H); MH+ 237.

### Method 8

### 2-Chloro-5-fluoro-4-(2-methyl-3-propan-2-yl-imidazol-4-yl)pyrimidine

5-Fluoro-4-(2-methyl-3-propan-2-yl-imidazol-4-yl)pyrimidin-2-ol acetate (Method 7; 4 g, 13.5 mmol) was suspended in phosphorus oxychloride (25 ml) and heated to 90° C. for 3.5 hrs. The reaction mixture was concentrated in vacuo then the residue dissolved in DCM (25 ml) and stirred with ice/water (50 ml). The mixture was cooled in an ice-water bath, neutralised to pH 8 with 40% aq NaOH then water and DCM (50 ml) were added and the organic layer separated. The aqueous layer was extracted with DCM (75 ml) then the combined organics were washed with brine, dried, filtered and evaporated to give a brown oil. Purification by flash chromatography on silica, eluting with 50% EtOAc/iso-hexane gave the title compound as a yellow oil which crystallised on standing (2.84 g, 83%). NMR (400.132 MHz, CDCl₃) 1.54 (d, 6H), 2.54 (s, 3H), 5.34 (m, 1H), 7.69 (m, 1H), 8.32 (m, 1H); MH+ 255.

### Method 9

### N—(Cyclopentyl-5-methyl-1,2-oxazol-4-amine

5-Methyl-1,2-oxazol-4-amine hydrochloride (20 g), cyclopentanone (13.9 ml) and sodium acetate (12.3 g) were added to MeOH (200 ml) and stirred at 0° C. for 1 hr. NaCNBH₃) (11.5 g) was slowly added over 20 mins, whilst maintaining the temperature below 0° C. After complete addition the reaction mixture was warmed to ambient temperature and stirred for 16 hrs before the solvent was removed in vacuo. The solid obtained was dissolved in saturated aq. NH₄Cl (100 ml) and extracted with ether (2×200 ml then 1×100 ml). The combined organic extracts were dried, filtered and the solvent removed in vacuo to give a yellow oil. The oil was purified by column chromatography on silica using 10-50% ether in isohexane as eluent. The solvent was removed in vacuo to give the title compound as a yellow oil (17.2 g). NMR (300.072 MHz, CDCl₃) 1.37-1.47 (m, 2H), 1.54-1.79 (m, 4H), 1.83-1.94 (m, 2H), 2.31 (s, 3H), 3.51 (quintet, 1H), 8.03 (s, 1H); m/z 167.

### Method 10

### N-Cyclopentyl-N-(5-methyl-1,2-oxazol-4-yl)acetamide

Acetic anhydride (18.9 ml) was added portionwise over 20 mins to a stirred solution of N-cyclopentyl-5-methyl-1,2-oxazol-4-amine (Method 9; 16.0 g) in acetic acid (160 ml). After 1 hr the solvent was removed in vacuo and the resulting slurry was treated with aqueous K₂CO₃ (50 ml, caution: CO₂ evolved). The aqueous layer was extracted with DCM (3×50 ml), the combined organics dried (Na₂SO₄) and the solvent was removed in vacuo. The solid obtained was dried under high vacuum to give the title compound as a yellow solid (19.0 g). NMR (300.074 MHz) 1.08-1.24 (m, 2H), 1.41-1.51 (m, 4H), 1.69 (s, 3H), 1.74-1.80 (m, 2H), 2.33 (s, 3H), 4.77 (quintet, 1H), 8.64 (s, 1H); MH+ 209.

### Method 11

### N—[(E)-1-Amino-3-oxo—but-1-en-2-yl]-N-cyclopentyl-acetamide

N-Cyclopentyl-N-(5-methyl-isoxazol-4-yl)-acetamide (Method 10; 19 g, 91 mmol)

was stirred with 10% palladium on carbon (3 g) in EtOH under a hydrogen atmosphere at increased pressure (4 atm.). The reaction was then filtered and the solvent removed in vacuo. DCM followed by ether was added to the residue and the reaction was filtered to give the title compound as a colourless solid (16 g, 84%). NMR (300.074 MHz) 1.19-1.49 (m, 6H), 1.59-1.80 (m, 5H), 2.06 (s, 3H), 4.44 (quintet, 1H), 6.84 (d, 2H), 7.59 (t, 1H); MH+ 211.

### Method 12

### 1-(3-Cyclopentyl-2-methyl-imidazol-4-yl)ethanone

N—[(E)-1-Amino-3-oxo—but-1-en-2-yl]-N-cyclopentyl-acetamide (Method 11; 16.0 g) and NaOH (3.66 g) were added to EtOH (200 ml) and heated under reflux for 4 hrs. NH₄Cl (6.11 g) was added and the mixture was stirred for 16 hrs at ambient temperature then concentrated in vacuo. Ether (350 ml) was added, the mixture stirred for 10 mins then filtered and concentrated in vacuo. The yellow oil obtained was distilled under reduced pressure (0.55 mbar/100° C.) to give the title compound as a clear oil (10.08 g). NMR (400.132 MHz, CDCl₃) 1.66-1.71 (m, 2H), 1.97-2.04 (m, 6H), 2.45 (s, 3H), 2.50 (s, 3H), 5.22 (quintet, 1H), 7.73 (s, 1H); MH+ 193.

### Method 13

### (E)-1-(3-Cyclopentyl-2-methyl-imidazol-4-yl)-3-dimethylamino-prop-2-en-1-one

1-(3-Cyclopentyl-2-methyl-imidazol-4-yl)ethanone (Method 12; 10.08 g) and DMF-DMA (17.9 ml) were added to DMF (150 ml) and heated at 130° C. for 6 hrs. The solvent was removed in vacuo and DCM (10 ml) was added followed by ether (100 ml). The mixture was sonicated for 10 mins before filtering and drying to give the title compound as a yellow solid (9.74 g). NMR (400.132 MHz, CDCl₃) 1.61-1.72 (m, 2H), 1.91-2.14 (m, 6H), 2.49 (s, 3H), 2.99 (s, 6H), 5.35 (quintet, 1H), 5.52 (d, 1H), 7.48 (s, 1H), 7.62 (d, 1H); MH+ 248.

### Method 14

### Benzyl 4-carbamimidamidopiperidine-1-carboxylate

Benzyl 4-aminopiperidine-1-carboxylate (20 g, 85.5 mmol) and TEA (24 ml, 171 mmol) were dissolved in acetonitrile (300 ml), then 1H-pyrazole-1-carboxamide (25 g, 171 mmol) was added and the reaction was heated at 65° C. for 16 hrs. The reaction mixture was then evaporated to dryness and quenched with sat. aq. sodium carbonate (200 ml), extracted with DCM (3×150 ml), combined organics dried and the solvent removed in vacuo to yield a orange solid. Acetonitrile (100 ml) was added and stirred at 65° C. for 20 mins, the resulting slurry was cooled and filtered to give the title compound as a colourless solid. NMR (400.132 MHz) 1.27-1.36 (m, 2H), 1.81-1.83 (m, 2H), 3.18-3.55 (m, 3H), 3.93 (d, 2H), 5.08 (s, 2H), 7.30-7.41 (m, 5H), 7.60-8.55 (brs, 4H); MH+ 277.

### Method 15

### tert-Butyl 3-carbamimidamidoazepane-1-carboxylate

Pyrazole-1-carboximidamide hydrochloride (739 mg, 5.04 mmol) was added to a stirred solution of tert-butyl 3-aminoazepane-1-carboxylate (541 mg, 2.52 mmol) and triethylamine (0.7 ml, 5.04 mmol) in acetonitrile (15 ml). The mixture was heated at reflux for 3.5 hrs then cooled to ambient temperature, filtered and washed with acetonitrile and dried under high-vacuum to give the title compound as a colourless solid (179 mg, 28%). The acetonitrile filtrate was evaporated in vacuo, the gum obtained was dissolved in DCM and sat. aq. sodium bicarbonate was added and the mixture was left to stand at ambient temperature for 16 hrs. The precipitated solid was filtered and dried in vacuo to give additional title compound as a colourless solid (192 mg, 30%). NMR (400.132 MHz) 1.41 (m, 9H), 1.68 (m, 3H), 3.37 (m, 8H), 7.81 (s, 2H).

### Example 211

The following illustrate representative pharmaceutical dosage forms containing the compound of formula (I), or a pharmaceutically acceptable salt or in vivo hydrolysable ester thereof (hereafter compound X), for therapeutic or prophylactic use in humans:—

| (a): Tablet I | mg/tablet |
|---|---|
| Compound X | 100 |
| Lactose Ph. Eur | 182.75 |
| Croscarmellose sodium | 12.0 |
| Maize starch paste (5% w/v paste) | 2.25 |
| Magnesium stearate | 3.0 |
| (b): Tablet II | mg/tablet |
| Compound X | 50 |
| Lactose Ph. Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |
| (c): Tablet III | mg/tablet |
| Compound X | 1.0 |
| Lactose Ph. Eur | 93.25 |
| Croscarmellose sodium | 4.0 |
| Maize starch paste (5% w/v paste) | 0.75 |
| Magnesium stearate | 1.0 |
| (d): Capsule | mg/capsule |
| Compound X | 10 |
| Lactose Ph. Eur | 488.5 |
| Magnesium stearate | 1.5 |
| (e): Injection I | (50 mg/ml) |
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | to 100% |
| (f): Injection II | 10 mg/ml |
| Compound X | 1.0% w/v |
| Sodium phosphate BP | 3.6% w/v |
| 0.1M Sodium hydroxide solution | 15.0% v/v |
| Water for injection | to 100% |
| | (1 mg/ml, |
| (g): Injection III | buffered to pH6) |
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

Note
The above formulations may be obtained by conventional procedures well known in the pharmaceutical art. The tablets (a)-(c) may be enteric coated by conventional means, for example to provide a coating of cellulose acetate phthalate.

1: A compound of formula (I): 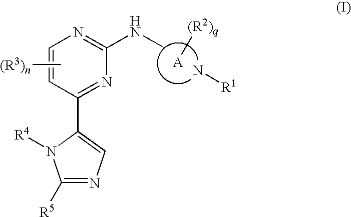
wherein:
Ring A is a 5-7 membered saturated heterocyclic ring which contains one nitrogen atom and optionally 1 or 2 additional heteroatoms selected from N, O or S; wherein 2 atoms of Ring A may optionally be connected by a bridge;
R¹ is a substituent on nitrogen and is selected from hydrogen, C₁₋₆alkyl, C₁₋₆alkanoyl, carbamoyl, N,N—(C₁₋₆alkyl)carbamoyl, N,N—(C₁₋₆alkyl)carbamoyl, N—(C₁₋₆alkenyl)carbamoyl, N,N—(C₁₋₆alkenyl)carbamoyl, sulphamoyl, N—(C₁₋₆alkyl)sulphamoyl, N,N—(C₁₋₆alkyl)₂sulphamoyl, N—(C₁₋₆alkenyl)sulphamoyl, N,N—(C₁₋₆alkenyl)sulphamoyl, C₁₋₆alkoxycarbonyl, C₁₋₆alkylsulphonyl, C₁₋₆alkenylsulphonyl, carbocyclyl-R⁶ or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;
R² is a substituent on carbon and is selected from halo, nitro, cyano, hydroxy, amino, carboxy, carbamoyl, mercapto, sulphamoyl, C₁₋₆alkyl. C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;
q is 0-4; wherein the values of R² may be the same or different;
R³ is selected from halo, cyano or amino;
n is 0 to 2, wherein the values of R³ may be the same or different;
R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;
R⁵ is selected from methyl, ethyl, isopropyl, fluoromethyl, difluoromethyl, trifluoromethyl, methoxymethyl, cyclopropylmethyl or cyclopropyl;
R⁶ and R⁷ are independently selected from —C(O)—, —C(O)N—(R¹⁵)—, —S(O)₂— or —SO₂N—(R¹⁶)—; wherein R¹⁵ and R¹⁶ are independently selected from hydrogen or C₁₋₆alkyl;
R⁸and R¹² are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R⁸ and R¹² independently of each other may be optionally substituted on carbon by one or more R¹⁹; and wherein if said heterocyclyl contains an —NH— moiety that nitrogen may be optionally substituted by a group selected from R²⁰;
R⁹, R¹³ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³ and R²⁰ independently of each other, may be optionally substituted on carbon by one or more R²¹;
R¹⁰, R¹¹, R¹⁷ and R¹⁸ are independently selected from a direct bond, —O—, —N—(R²²)—, —C(O)—, —N—(R²³)C(O)—, —C(O)N—(R²⁴)—, —S(O)ₛ—, —SO₂N—(R²⁵)— or —N—(R²⁶⁾SO₂—; wherein R²², R²³, R²⁴, R²⁵ and R²⁶ are independently selected from hydrogen or C₁₋₆alkyl and s is 0-2;
R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino; and
R¹⁹ and R²¹ are independently selected from halo, nitro, cyano, hydroxy, trifluoromethoxy, trifluoromethyl, amino, carboxy, carbamoyl, mercapto, sulphamoyl, methyl, ethyl, propyl, isopropyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt or an in vivo hydrolysable ester thereof.

2: A compound of formula (I), or a pharmaceutically acceptable salt or an in vivo hydrolysable ester thereof, as claimed in claim 1 wherein Ring A is azepan-3-yl, 3-azabicyclo[3.1.0]hexan-3-yl, piperidin-3-yl, piperidin-4-yl, pyrrolidin-3-yl or 8-azabicyclo[3.2.1]octan-3-yl.

3: A compound of formula (I), or a pharmaceutically acceptable salt or an in vivo hydrolysable ester thereof, as claimed in claim 1 wherein R¹ is a substituent on nitrogen and is selected from hydrogen, C₁₋₆alkyl, C₁₋₆alkanoyl, carbamoyl, N—(C₁₋₆alkyl)carbamoyl, N,N—(C₁₋₆alkyl)carbamoyl, sulphamoyl, N—(C₁₋₆alkyl)sulphamoyl, N,N—(C₁₋₆alkyl)₂sulphamoyl, C₁₋₆alkoxycarbonyl, C₁₋₆alkylsulphonyl, C₁₋₆alkenylsulphonyl or heterocyclyl-R⁷; wherein R¹ may be optionally substituted on carbon by one or more R⁸; and wherein if said heterocyclyl contains an —NH— moiety that nitrogen may be optionally substituted by a group selected from R⁹;
R⁷ is selected from —C(O)—, —C(O)N—(R¹⁵)—, —S(O)₂— or —SO₂N—(R¹⁶)—; wherein R¹⁵ and R¹⁶ are hydrogen;
R⁸ is selected from halo, nitro, hydroxy, amino, C₁₋₆alkyl, C₁₋₆alkoxy, N—(C₁₋₆alkyl)amino, N,N—(C₁₋₆alkyl)₂amino, carbocyclyl-R¹⁷— or heterocyclyl-R¹⁸—; wherein R⁸ may be optionally substituted on carbon by one or more R¹⁹; and wherein if said heterocyclyl contains an —NH— moiety that nitrogen may be optionally substituted by a group selected from R²⁰;
R⁹ and R²⁰ are independently selected from C₁₋₆alkyl, C₁₋₆alkanoyl, C₁₋₆alkoxycarbonyl and benzyloxycarbonyl; wherein R⁹ and R²⁰ independently of each other, may be optionally substituted on carbon by one or more R²¹;
R¹⁷ and R¹⁸ are independently selected from a direct bond or —N—(R²²)—; wherein R²² is selected from hydrogen or C₁₋₆alkyl; and
R¹⁹ and R²¹ are independently selected from halo, cyano, hydroxy, carbamoyl, methyl, propyl, cyclopropyl and methoxy.

4: A compound of formula (I), or a pharmaceutically acceptable salt or an in vivo hydrolysable ester thereof, as claimed in claim 1 wherein q is 0.

5: A compound of formula (I), or a pharmaceutically acceptable salt or an in vivo hydrolysable ester thereof, as claimed in claim 1 wherein R³ is halo.

6: A compound of formula (I), or a pharmaceutically acceptable salt or an in vivo hydrolysable ester thereof, as claimed in claim 1 wherein n is 0 or 1.

7: A compound of formula (I), or a pharmaceutically acceptable salt or an in vivo hydrolysable ester thereof, as claimed in claim 1 wherein R⁴ is selected from isopropyl or cyclopentyl.

8: A compound of formula (I), or a pharmaceutically acceptable salt or an in vivo hydrolysable ester thereof, as claimed in claim 1 wherein R⁵ is methyl.

9: A compound of formula (I): 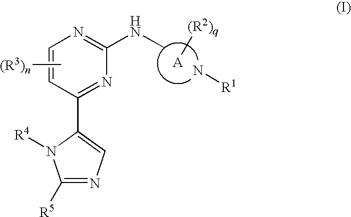
wherein:
Ring A is azepan-3-yl, (1α, 5α, 6α)-3-azabicyclo[3.1.0]hexan-3-yl, (R)-piperidin-3-yl, (S)-piperidin-3-yl, piperidin-4-yl, pyrrolidin-3-yl or endo-8-azabicyclo[3.2.1]octan-3-yl;
R¹ is a substituent on nitrogen and is selected from hydrogen, methyl, propyl, isopropyl, ethenylsulphonyl, mesyl, benzyloxycarbonyl, t-butoxycarbonyl, acetyl, phenethyl, ethoxycarbonyl, 2-methoxyethyl, sulphamoyl, N,N-dimethylsulphamoyl, N,N-dimethylcarbamoyl, benzyl, carbamoyl, N-methylcarbamoyl, 2-(dimethylamino)ethylsulphonyl, 2-(N-methyl-N-isopropyl)ethylsulphonyl, 2-(1-methylpiperazin-4-yl)ethylsulphonyl, 2-pyrrolidin-1-ylethylsulphonyl, 2-(3-fluoropyrrolidin-1-ylethylsulphonyl, 2-(thiomorpholin-4-yl)ethylsulphonyl, 2-(4-methylpiperidin-1-yl)ethylsulphonyl, 2-(homopiperidin-1-ylethylsulphonyl, 2-diethylaminoethylsulphonyl, 2-azetidin-1-ylethylsulphonyl, 2-morpholinoethylsulphonyl, 2-(4-fluoropiperidin-1-yl)ethylsulphonyl, 2-(4-cyanopiperidin-1-ylethylsulphonyl, 2-(4-propylpiperidin-1-ylethylsulphonyl, 2-(4-carbamoylpiperidin-1-ylethylsulphonyl, 2-(7-azabicyclo[2.2.1]hept-7-yl)ethylsulphonyl, 2-(2-azabicyclo[2.2.2]oct-2-yl)ethylsulfonyl, 2-(6-azabicyclo[2.2.2]oct-6-yl)ethylsulfonyl, 2-homomorpholinoethylsulphonyl, 2-(2-oxopiperazin-4-yl)ethylsulphonyl, 2-(1-acetylpiperazin-4-yl)ethylsulphonyl, 2-(2-methoxyethylamino)ethylsulphonyl, 2-(N-methyl-N-cyclopropyl)ethylsulphonyl, 2-(2-oxohomopiperazin-4-yl)ethylsulphonyl, 2-(1-acetylhomopiperazin-4-yl)ethylsulphonyl, 2-(N-methyl-N-cyclopropylmethyl)ethylsulphonyl 2-(homothiomorpholin-4-yl)ethylsulphonyl, 3-chloropropylsulphonyl, 3-dimethylaminopropylsulphonyl, 3-dimethylamino-2,2-dimethylpropylsulphonyl, 3-diethylaminopropylsulphonyl, 3-(2-methoxyethylamino)propylsulphonyl, 3-[N-methyl-N-(2-methoxyethyl)amino]propylsulphonyl, 3-hydroxypropylsulphonyl, 3-(1-hydroxyprop-2-ylamino)propylsulphonyl, 3-(1-methylpiperazin-4-yl)propylsulphonyl, 3-(1-isopropylpiperazin-4-yl)propylsulphonyl, 3-(6-azabicyclo[2.2.2]oct-6-yl)propylsulfonyl, 3-(7-azabicyclo[2.2.1]hept-7-yl)propylsulphonyl, 3-[1-(2-hydroxyethyl)piperazin-4-yl]propylsulphonyl, 3-pyrrolidin-1-ylpropylsulphonyl, 3-(1,4-dimethylpyrrolidin-1-yl)propylsulphonyl, 3-morpholinopropylsulphonyl, 3-(1-hydroxybut-2-ylamino)propylsulphonyl, 3-(1-methoxyprop-2-ylamino)propylsulphonyl, 3-(2-hydroxypropylamino)propylsulphonyl, 3-(1-hydroxy-3-methylbut-2-ylamino)propylsulphonyl, 3-(1-hydroxy-2-methylprop-2-ylamino)propylsulphonyl, 3-(piperidin-1-yl)propylsulphonyl, 3-(cyclopropylamino)propylsulphonyl, 3-(N-methyl-N-cyclopropylamino)propylsulphonyl, 3-(cyclopentylamino)propylsulphonyl, 3-(N-methyl-N-cyclopentylamino)propylsulphonyl, 3-(cyclobutylamino)propylsulphonyl, 3-(N-methyl-N-cyclobutylamino)propylsulphonyl, 3-(isopropylamino)propylsulphonyl, 3-(N-methyl-N-isopropylamino)propylsulphonyl, 3-(N-methyl-N-ethylamino)propylsulphonyl, 3-[N-methyl-N-(2-cyanoethyl)amino]propylsulphonyl, 3-[N-methyl-N-(2-cyanoethyl)amino]propylsulphonyl, 3-azetidin-1-ylpropylsulphonyl, 3-(cyclopropylmethylamino)propylsulphonyl, 3-(N-methyl-N-cyclopropylmethylamino)propylsulphonyl, 3-nitro-3-methylbutylsulphonyl, 3-amino-3-methylbutylsulphonyl, 3-dimethyl-3-methylbutylsulphonyl, 2-(piperidin-3-yl)acetyl, 2-(1--butoxycarbonylpiperidin-3-yl)acetyl, 2-(piperidin-4-yl)acetyl, 2-(1-t-butoxycarbonylpiperidin-4-yl)acetyl, 2-dimethylaminoacetyl, 3-(1-t-butoxycarbonylpiperazin-4-yl)propanoyl, 3-(1-t-butoxycarbonylpiperidin-4-yl)propanoyl, 3-(piperidin-4-yl)propanoyl. 3-(piperazin-4-yl)propanoyl, 3-dimethylaminopropanoyl, 4-morpholinobutanoyl, 4-dimethylaminobutanoyl, 1-t-butoxycarbonyl-4-methylpiperidin-4-ylcarbonyl, 4-methylpiperidin-4-ylcarbonyl, 1-t-butoxycarbonyl-4-methylpiperidin-4-ylcarbonyl, 4-methylhomopiperazin-1-ylcarbonyl, 1-methylpiperidin-3-ylcarbonyl, 1-methylpyrrolidin-2-ylcarbonyl, 3-dimethylaminopyrrolidin-1-ylcarbonyl, 4−/−butoxycarbonylmorpholin-2-ylcarbonyl, morpholin-2-ylcarbonyl, 1-methylpiperidin-4-ylcarbamoyl, N-(1-ethylpyrrolidin-2-ylmethyl)carbamoyl, N-(2-pyrrolidin-1-ylethyl)carbamoyl, N-(2-dimethylaminoethyl)carbamoyl, N-(1-methylpiperidin-4-yl)sulphamoyl, N-(1-isopropylpiperidin-4-yl)sulphamoyl, 2-(dimethylamino)ethylsulphamoyl, 2-(diethylamino)ethylsulphamoyl, 2-(morpholino)ethylsulphamoyl, 2-(1-methylpiperazin-4-yl)ethylsulphamoyl, 2-(1-methylpyrrolidin-2-yl)ethylsulphamoyl, 3-(pyrrolidin-1-yl)propylsulphamoyl, 3-(3-fluoropyrrolidin-1-yl)propylsulphamoyl, 3-dimethylamino-2,2-dimethylpropylsulphamoyl, 3-(piperidin-1-yl)propylsulphamoyl, N-methyl-N-(3-dimethylaminopropyl)sulphamoyl, 3-dimethylaminopyrrolidin-1-ylsulphonyl, 1-methylpiperazin-4-ylsulphonyl, 1-methylpiperidin-4-ylsulphonyl, 1-isopropylpiperidin-4-ylsulphonyl and 1-methylhomopiperazin-4-ylsulphonyl;
q is 0;
R³ is fluoro or chloro;
n is 0 or 1;
R⁴ is selected from isopropyl or cyclopentyl;
R⁵ is methyl;
or a pharmaceutically acceptable salt or an in vivo hydrolysable ester thereof.

10: A compound of formula (I): 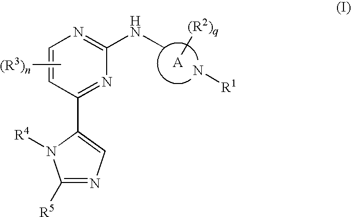
selected from:
5-fluoro-4-(2-methyl-3-propan-2-yl-imidazol-4-yl)-N-(1-methylsulphonyl-4-piperidinyl)pyrimidin-2-amine;
5-fluoro-4-(2-methyl-3-propan-2-yl-imidazol-4-yl)-N-[1-(2-pyrrolidin-1-ylethylsulphonyl)-4-piperidinyl]pyrimidin-2-amine;
N-[1-(3-dimethylamino-3-methyl-butyl)sulphonyl-4-piperidinyl]-5-fluoro-4-(2-methyl-3-propan-2-yl-imidazol-4-yl)pyrimidin-2-amine;
N-[1-(3-chloropropylsulphonyl)-4-piperidinyl]-5-fluoro-4-(2-methyl-3-propan-2-yl-imidazol-4-yl)pyrimidin-2-amine;
5-fluoro-4-(2-methyl-3-propan-2-yl-imidazol-4-yl)-N-[1-(3-pyrrolidin-1-ylpropylsulphonyl)-4-piperidinyl]pyrimidin-2-amine;
5-fluoro-4-(2-methyl-3-propan-2-yl-imidazol-4-yl)-N-[1-[3-(1-piperidinyl)propylsulphonyl]-4-piperidinyl]pyrimidin-2-amine;
N-[1-[2-(6-azabicyclo[2.2.2]oct-6-yl)ethylsulfonyl]-4-piperidinyl]-5-fluoro-4-(2-methyl-3-propan-2-yl-imidazol-4-yl)pyrimidin-2-amine;
N-[1-[3-(6-azabicyclo[2.2.2]oct-6-yl)propylsulfonyl]-4-piperidinyl]-5-fluoro-4-(2-methyl-3-propan-2-yl-imidazol-4-yl)pyrimidin-2-amine;
N-[1-[3-(7-azabicyclo[2.2.1]hept-7-yl)propylsulphonyl]-4-piperidinyl]-5-fluoro-4-(2-methyl-3-propan-2-yl-imidazol-4-yl)pyrimidin-2-amine; and
5-fluoro-4-(2-methyl-3-propan-2-yl-imidazol-4-yl)-N-[1-[(1-propan-2-yl-4-piperidinyl)sulfonyl]-4-piperidinyl]pyrimidin-2-amine;
or a pharmaceutically acceptable salt or an in vivo hydrolysable ester thereof.

11: A process for preparing a compound of formula (I) or a pharmaceutically acceptable salt or an in vivo hydrolysable ester thereof, as claimed in claim 1, which process, wherein variable groups are, unless otherwise specified, as defined in claim 1, comprises:
Process a) reacting a pyrimidine of formula (II): 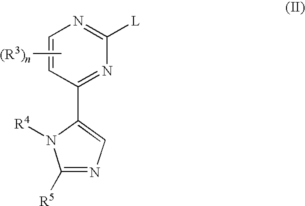
wherein L is a displaceable group; with an amine of formula (III): 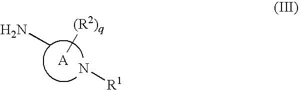
or
Process b) reacting a compound of formula (IV): 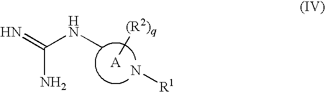
with a compound of formula (V): 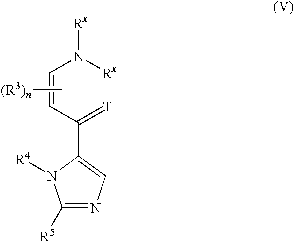
wherein T is O or S; R^{x} may be the same or different and is selected from C₁₋₆alkyl; or
Process c) reacting a pyrimidine of formula (VI): 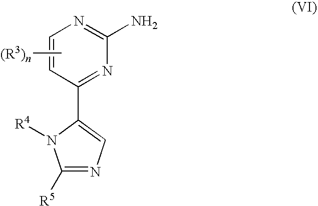
with a compound of formula (MI): 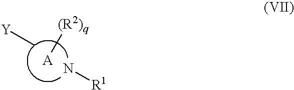
where Y is a displaceable group;
and thereafter optionally:
i) converting a compound of the formula (I) into another compound of the formula (I);
ii) removing any protecting groups;
iii) forming a pharmaceutically acceptable salt or in vivo hydrolysable ester.

12: A pharmaceutical composition which comprises a compound of the formula (I), or a pharmaceutically acceptable salt or in vivo hydrolysable ester thereof, as claimed in any one of claims 1, 9, 10 and a pharmaceutically-acceptable diluent or carrier.

13: A compound of the formula (I), or a pharmaceutically acceptable salt or in vivo hydrolysable ester thereof, as claimed in any one of claims 1, 9, 10, for use as a medicament.

14-18. (canceled)

19: A method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

20: A method of producing a CDK2 inhibitory effect, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

21: A method of treating cancer, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

22: A method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

23: A method of treating cancer, fibroproliferative and differentiative disorders, psoriasis, rheumatoid arthritis. Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.
